SEQUENCE LISTING (5 sequences)

SEQ ID NO: 1 - RNA, 1045 nt, synthetic construct
  source 1..1045: mol_type = other RNA
ttaaagaaaattctcaggccaggctcaatggctcatgcctataattgtagtgctttgggaggctgagctgggaggatcgcttaagaccagcctaggcaacagagtgagatcctgtctctacaaaaacaaaaattttttttttgttttctttttgagacggagtttcgctcttgttgcccaggctggagtgcaatggtgcggtcttggctcaccgcaacctccgcctcccagattaaagtgattctcctgcctcaacctcctgagtagctgggattacaggcatgcgccaccatgccgggcgaattttgtatttttaatagagacagggtttctctgtgttggtcaggctggtctcaaactcccaacctcaggtgatccgcccacctcagcctctcagagtgctgggattacagatgtgagccactgcacctggcaaaaaaaaaatttttttttaatttgtcaggtgtggtgatgcatgcatgtagtcccaaatacttaggaggctgaggtggaaggatcacttgagcccaggaggtcaaggcaacagtgagctgtgatcatgccactgcactccagtctgggtgacacagtgagaccctgtctcttaaaaaaaaaaaaagaaagaaaaaaaattattctttaggtaatttcacttggacatattcatgtatatggagtttgtcagctgactcaggcatttaccctaaagatcaaatctgttttcaaaaattggcattggccaggcacggtgacccacacctgtaatcccagcactttgggaggccgaggcgggcggatcacgaggtcaggggttcaagaccagcctggccaacatagtgaaaccccgtctctactaaaaatacaaaaaattagctgggcatggtggtgggcgcctgtaatcccagctacttgagaggctcaggcaggagaatcgcttgaacctgggagtggaggttgcagtgagccgagattgcaccattgtattccaccccgggtgacagtgcaagactccatctcaaaaaaaaaaaaaaaatttggcatcatt

SEQ ID NO: 2 - RNA, 1045 nt, synthetic construct
  source 1..1045: mol_type = other RNA
  modified_base 94: mod_base = i
ttaaagaaaattctcaggccaggctcaatggctcatgcctataattgtagtgctttgggaggctgagctgggaggatcgcttaagaccagcctnggcaacagagtgagatcctgtctctacaaaaacaaaaattttttttttgttttctttttgagacggagtttcgctcttgttgcccaggctggagtgcaatggtgcggtcttggctcaccgcaacctccgcctcccagattaaagtgattctcctgcctcaacctcctgagtagctgggattacaggcatgcgccaccatgccgggcgaattttgtatttttaatagagacagggtttctctgtgttggtcaggctggtctcaaactcccaacctcaggtgatccgcccacctcagcctctcagagtgctgggattacagatgtgagccactgcacctggcaaaaaaaaaatttttttttaatttgtcaggtgtggtgatgcatgcatgtagtcccaaatacttaggaggctgaggtggaaggatcacttgagcccaggaggtcaaggcaacagtgagctgtgatcatgccactgcactccagtctgggtgacacagtgagaccctgtctcttaaaaaaaaaaaaagaaagaaaaaaaattattctttaggtaatttcacttggacatattcatgtatatggagtttgtcagctgactcaggcatttaccctaaagatcaaatctgttttcaaaaattggcattggccaggcacggtgacccacacctgtaatcccagcactttgggaggccgaggcgggcggatcacgaggtcaggggttcaagaccagcctggccaacatagtgaaaccccgtctctactaaaaatacaaaaaattagctgggcatggtggtgggcgcctgtaatcccagctacttgagaggctcaggcaggagaatcgcttgaacctgggagtggaggttgcagtgagccgagattgcaccattgtattccaccccgggtgacagtgcaagactccatctcaaaaaaaaaaaaaaaatttggcatcatt

SEQ ID NO: 3 - RNA, 30 nt, synthetic construct
  source 1..30: mol_type = other RNA
agcctaggcaacagagtgagatcctgtctc

SEQ ID NO: 4 - RNA, 30 nt, synthetic construct
  source 1..30: mol_type = other RNA
  modified_base 6: mod_base = i
  modified_base 10..11: mod_base = i
  modified_base 13: mod_base = i
  modified_base 15: mod_base = i
  modified_base 19: mod_base = i
agcctnggcnncngngtgngatcctgtctc

SEQ ID NO: 5 - DNA, 20 bp, synthetic construct
  misc_feature 6..15: note = DNA
  source 1..20: mol_type = other DNA
  misc_feature 1..5: note = RNA
  misc_feature 16..20: note = RNA
  modified_base 1..5: mod_base = OTHER; note = 2MOE
  modified_base 16..20: mod_base = OTHER; note = 2MOE
  modified_base 19: mod_base = OTHER; note = thymine
agaaacacctgcggcggctc

(19) United States
(12) Patent Application Publication
(10) Pub. No.: US 2026/0083766 A1
(43) Pub. Date: Mar. 26, 2026

(54) METHODS TO TREAT NEURODEGENERATIVE DISORDERS BY REDUCING NUCLEAR AGGREGATES AND MODULATING ADENOSINE-TO-INOSINE RNA EDITING

(71) Applicant: Northwestern University, Evanston, IL (US)

(72) Inventors: Nandkishore Raghav Belur, Evanston, IL (US); Joseph Robert Mazzulli, Chicago, IL (US)

(21) Appl. No.: 19/211,071

(22) Filed: May 16, 2025

Related U.S. Application Data
(60) Provisional application No. 63/648,407, filed on May 16, 2024.

(51) Int. Cl. A61K 31/7064; A61P 25/16; A61P 25/28; C12N 15/113

(52) U.S. Cl. CPC A61K 31/7064; A61P 25/16; A61P 25/28; C12N 15/113; C12N 2310/11

(57) ABSTRACT

This disclosure provides methods for treating neurodegenerative diseases and restoring neuronal synaptic function by administering adenosine-to-inosine editing modifiers.

### CROSS-REFERENCE TO RELATED APPLICATIONS

This application claims priority to U.S. Provisional Application No. 63/648,407, filed May 16, 2024. The contents of which is incorporated by reference in its entirety.

### STATEMENT REGARDING FEDERALLY SPONSORED RESEARCH

This invention was made with government support under grant numbers NS118824 and NS107768 awarded by the National Institutes of Health. The government has certain rights in this invention.

### REFERENCE TO AN ELECTRONIC SEQUENCE LISTING

The contents of the electronic sequence listing (70258102669.xml; Size: 12,316 bytes; and Date of Creation: May 16, 2025) is herein incorporated by reference in its entirety.

### BACKGROUND

The accumulation of aggregated proteins and failure of protein homeostasis (proteostasis) in neurons is thought to underlie cell death in age-related neurodegenerative disorders. In Parkinson's disease (PD) and Dementia with Lewy Bodies (DLB), proteinaceous inclusions termed Lewy bodies and Lewy neurites are comprised of the pre-synaptic protein α-synuclein (α-syn) that histopathologically define the disease. Lewy inclusions were initially identified as eosinophilic bodies that strongly react with anti-ubiquitin or neurofilament antibodies, however the identification of aggregation-promoting SNCA gene mutations in rare familial PD led to the discovery of α-syn as a Lewy body component and the classification of PD and DLB as synucleinopathies. The accumulation of filamentous α-syn and co-occurrence with neurofilament in axon terminals is considered a major pathology that is closely associated with synaptic degeneration and loss of function at nerve terminals. Early degeneration of synapses and axons is an initial key step in pathophysiology that occurs prior to cell body loss, however the mechanisms that trigger degeneration at axon terminals are not understood.

Recent genetic studies of common sporadic forms of PD and DLB have expanded our understanding of the pathophysiology beyond α-syn, establishing dysfunctional cellular degradation pathways as potential disease drivers that promote pathologic inclusions. Mutations in lysosomal GBA1 gene are the strongest link known to date, and other variants have been identified in over 15 additional lysosomal hydrolases, channel proteins, or trafficking machinery with distinct degradative functions. Furthermore, the accumulation of α-syn alone can directly disable multiple proteostasis pathways, including protein folding, maturation and degradation by lysosomes. Thus, an intrinsic relationship exists between pathological α-syn aggregates and genetic pathways in PD and DLB, suggesting that proteostasis failure may be a primary underlying cause of disease. Despite this, it is unclear how impairment of a general proteostasis pathway can result in the putative specific pathology that defines these diseases. Accordingly, there is a remaining need in the art for understanding the mechanism by which aggregates affect neurodegeneration, and for strategies to prevent neurodegeneration.

### SUMMARY

The present disclosure provides methods for treating neurodegenerative diseases including dissolving and inhibiting pathological aggregates and restoring neuronal synaptic function. The inventors demonstrate an increase in A to I RNA editing promotes aggregation of proteins and polynucleotides. The inventors found that inhibiting A to I RNA editing in these cells dissolves pathological aggregates and restores neuronal synaptic health.

One aspect of the present disclosure provides a method of treating a neurodegenerative disease in a subject in need thereof, the method comprising administering an inhibitor of Adenosine (A) to Inosine (I) RNA editing to the subject. In some embodiments, the inhibitor comprises at least one of: an ADAR1 inhibitor; an ADAR2 inhibitor; and an ADAR3 activator. In some embodiments the ADAR1 inhibitor inhibits at least one of ADAR1 expression and/or activity. In some embodiments, the ADAR1 inhibitor is a small-molecule drug. In some embodiments, the ADAR1 inhibitor comprises 8-aza-adenosine (8-aza). In some embodiments, the 8-aza is administered at a sub-toxic dose. In some embodiments, the 8-aza is administered at less than about 1 μM. In some embodiments, the 8-aza is administered between about 0.05 μM and about 1 μM. In some embodiments, the 8-aza is administered at about 200 nM. In some embodiments, the neurodegenerative disease is selected from the group consisting of Parkinson's disease, Alzheimer's disease, dementia with Lewy bodies and multiple system atrophy.

A second aspect of the present disclosure provides a method of preventing or dissolving pathological aggregates comprising at least one of NonPOU Domain-Containing Octamer-Binding Protein (NONO) and Splicing Factor, Proline- and Glutamine-Rich (SFPQ) in a cell, the method comprising contacting the cell with an inhibitor of adenosine (A) to inosine (I) RNA editing. In some embodiments, the inhibitor comprises at least one of: an ADAR1 inhibitor; an ADAR2 inhibitor; and an ADAR3 activator. In some embodiments, the ADAR1 inhibitor inhibits at least one of ADAR1 expression and activity. In some embodiments, the ADAR1 inhibitor comprises 8-aza. In some embodiments, the cell is contacted with less than about 1 μM 8-aza. In some embodiments, the cell is contacted with about 200 nM 8-aza. In some embodiments, the cell is an induced pluripotent stem neuronal (iPSn) cell derived from a subject having a neurodegenerative disease.

Another aspect of the present disclosure provides a method of treating a neurodegenerative disease or a neurodevelopmental disorder in a subject in need thereof, the method comprising administering an inhibitor of NEAT1_2 to the subject. In some embodiments, the NEAT1_2 inhibitor is 8-aza or an antisense oligonucleotide.

### BRIEF DESCRIPTION OF THE DRAWINGS

The patent or application file contains at least one drawing executed in color. Copies of this patent or patent application with color drawing(s) will be provided by the Office upon request and payment of the necessary fee.

The present technology can be better understood by reference to the following drawings. The drawings are merely exemplary to illustrate certain features that may be used singularly or in combination with other features and the present technology should not be limited to the embodiments shown.

FIGS. 1A-1G. Proteomic analysis of aggregated proteins in PD midbrain cultures. A) Midbrain cultures were labeled to perform SILAC-based mass spectrometry on soluble and insoluble fractions. Quadrant 1 represents proteins that shift from soluble into insoluble fractions in A53T α-syn iPSn compared to isogenic controls. Gray plots are non-significant, while white plots are significant compared to isogenic corrected lines (FDR adjusted p-value <0.05). Proteins outside of the dashed box are changed more than 2-fold and were prioritized. B) Plots of individual fold changes of protein levels obtained from SILAC labeled A53T iPSn at day 60 and day 90 compared to corrected lines (dashed line). Each plot represents an individual culture well (n=4, *p<0.05, FDR adjusted for multiple comparisons). Examples of control proteins with subtle changes are shown on the right side of the graph, demonstrating selectivity. C) Enrichment analysis of proteins with reduced solubility in d90 PD iPSn (orange shade in panel A) by biological process (left) or reactome (right) reveals overrepresentation and network associations of aggregated proteins. Each node represents an individual functional category; node size correlates to number of genes in each category; node color indicates P-value. D)-G) Correlation of aggregated proteins with primary amino acid sequence or secondary structure.

FIGS. 2A-2E. Nuclear aggregation of NONO/SFPQ in PD iPSn and post-mortem synucleinopathy brain. A) Sequential extraction/western blot analysis of NONO, SFPQ in day 60 and 90 patient iPSn compared to isogenic controls (Corr), quantified on the right (n=4 culture wells). Data is quantified as Insolubility normalized to CBB (decline in soluble and increase in insoluble fractions). CBB (Coomassie brilliant blue), GAPDH, and βiii-Tubulin are loading controls. B) Fixed iPSn (d90) were immunostained for NONO/SFPQ. A53T iPSn were imaged by confocal microscopy, showing 2 focal planes of the same cell (A53T-1, -2) to better reveal puncta. TH, tyrosine hydroxylase stain indicates accumulation in midbrain neurons. Scale bar=10 μm. Quantification is shown below for each separate protein or puncta that are colocalized (n=4 culture wells). C) Subcellular fractionation/western blot of d90 iPSn (n=3 culture wells). D) Sequential extraction/western blot analysis of human frontal cortex from healthy controls, Dementia with Lewy body (DLB), Alzheimer's disease (AD) or progressive supranuclear palsy (PSP) patients. CBB, Coomassie brilliant blue (load control). Right, Quantification of insolubility normalized to total protein (CBB) (n=4 controls, n=8 DLB, AD, or PSP), linked to FIG. 10B. Each plot represents a measure from an individual brain sample. E) Immunohistological analysis of human frontal cortex of DLB patients. Secondary (2ary) antibody alone is a negative control. Nuclei were stained with DAPI. Scale bar, 10 μm. Quantification on the right; each plot represents the average number of large inclusions per brain, from 4 individual brains. All values are mean±SEM, *p<0.05; **p<0.01; ****p<0.0001, using student's unpaired t-test (A, B, C, E). ANOVA with Dennett's post-hoc test was used for panel D.

FIGS. 3A-3K. Interaction and co-aggregation of α-syn oligomers with SFPQ. A) Proximity ligation assay of α-syn with either NONO or SFPQ in PD iPSn (A53T α-syn), quantified on the right (each plot represents an individual culture well). B) Confocal microscopy analysis showing co-localization of α-syn/NONO/SFPQ in the nucleus of PD iPSn through one individual 1 um focal plane as a representative example. Colocalization was quantified on the right (each plot represents an individual culture well). C) Purified recombinant SFPQ (40 kDa truncated form) was mixed with recombinant α-syn monomers (mon.) oligomers (olig.) or pre-formed fibrils (PFFs) in vitro (each at 1 mg/ml) and aggregation was assessed by sedimentation/western blot for SFPQ (C) or α-syn(D) (detected on the same membrane with different secondary channels). Asterisk indicates residual signal from BSA. Reactions were separated into supernatant (S) or 100,000×g pellet (P) fractions. High exposures (exp.) are shown to better reveal high molecular weight (HMW) forms of SFPQ (C) or α-syn(D). E) Both SFPQ and α-syn were detected on the same membrane with distinct labeled secondary antibodies, shown by colocalization of the signals from panels C and D. F) Quantification of pelleted proteins and HMW SFPQ at 24 hr (from 58 kDa to 123 kDa). G) Western blot analysis of SFPQ/α-syn co-aggregation rate from 0-48 hrs reveals initial stabilization of soluble HMW SFPQ by α-syn oligomers, followed by aggregation into pelleted fractions between 24 and 48 hrs. Asterisk indicates residual signal from BSA. H) Quantification of the SFPQ signal in grey plots or α-syn signal in yellow plots. SFPQ+α-syn oligomers followed a synchronized aggregation rate (red dotted lines). I) Quantification of HMW SFPQ in supe or pellet fractions at 48 hr. J) Incubations from 48 hr were separated into supe or pellet fractions followed by thioflavin T (ThT) reactivity (RFU, relative fluorescence units). K) High Salt (HS) soluble fractions from controls or DLB brains were analyzed by western blot, revealing soluble HMW SFPQ in vivo. Right, Quantification indicates values from three separate controls or DLB brains. All values are mean±SEM, *p<0.05; **p<0.01; ****p<0.0001, using student's unpaired t-test for A, B, and K. ANOVA with Tukey's test was used for F, H, I, and J.

FIGS. 4A-4K. Reduced ADAR3 expression and increased A-to-I editing in synucleinopathy patient material. A) Isogenic corrected (Corr) or A53T iPSn were transfected with a Renilla luciferase construct driven by the ADAR3 promoter, and normalized to a co-transfected construct expressing SV-40 driven Firefly luciferase. Black and white datapoints represent two independent passages, analyzed 48 hrs post-transfection (n=6-10) B) mRNA was quantified by Q-RT-PCR in patient A53T and isogenic corrected (Corr) iPSn at day 60 and d90 (n=3 culture wells). C) Western blot analysis of ADAR3 in iPSn (cultures matching with panel B, d90) shows near complete depletion of ADAR3. Right, quantification (n=3 culture wells). D) Western blot analysis of ADAR3 levels in human brain frontal cortex from healthy controls (ctrl) and Dementia with Lewy bodies (DLB) patients. Right, quantification normalized to CBB. E) Quantification of CYFIP2 and CADPS protein and mRNA by SILAC-MS or RT-PCR, respectively at d60. F) Sequencing chromatograms showing the edited region of CYFIP2 transcript in genomic DNA (gDNA) and complementary DNA (cDNA) for corr and A53T iPSn at day 60. The red arrow indicates the site of A-to-I edit in cDNA. Right, quantification of percent edit of CYFIP2 site in Corr compared to A53T iPSn. G) Percent edit of CADPS in Corr compared to A53T iPSn at d60 assessed by Sanger sequencing. H) Nuclei were isolated from GFP or ADAR3 lenti-infected A53T iPSn (d90) were isolated and A-to-I editing was assessed as in panel F. I) Correlation between percentage of edited CYFIP2 and age (years) in ctrl and DLB human frontal cortex samples. Each data point represents a measure of an individual patient. J) Quantification showing percentage edit of CYFIP2 from all the ctrl and LBD patient samples in panel I. K) Quantification of percentage edited CYFIP2 shown in panel I based on age (>65 years) and pathological diagnosis of patient samples (BLBD—Brainstem Lewy Body Disease, DLB SNCA mt—with α-synuclein mutations). All values are the mean±SEM, *p<0.05; **p<0.01, ***p<0.001, ****p<0.0001, using student's unpaired t-test (panel A, C-H, J) or ANOVA with Tukey's post-hoc test (panel B, K). Plots represent either an individual culture well or an individual brain sample. See FIG. 12 for more information.

FIGS. 5A-5F. RNA-seq of PD iPSn reveals increased A-to-I edit sites that correlate with decreased protein expression within synapse, axon, and mitochondria categories. A) Quantification of RNA A-I edit sites that are increased in PD (A53T) iPSn (orange) or isogenic controls (blue) at day 60. Below, assessment of average % increase in A-I editing within the 3,137 sites found elevated in PD iPSn. Each plot represents an individual edit site (avg from 4 culture wells). B) Heat map analysis of increased edit sites in PD (left) or controls (right). Each column represents an individual culture well from control (C) or A53T mutant (M), while each row represents individual edit sites identified that are significantly changed. Inset, the top 50 hyper-edited hits in PD iPSn with gene names shown on the left. C) Heat map of significantly changed RNA edit sites clustered by transcript location. The number of sites are indicated above each bar graph. D) Heat map of significantly changed RNA edit sites clustered by transcript type reveals that human specific Alu regions are most prominently affected. E) Enrichment analysis of edit sites that are significantly elevated by >10% in PD iPSn by cell component. Each node represents an individual functional category; node size correlates to number of genes in each category; node color indicates P-value. F) Correlation of changes in A-I editing with protein levels detected by SILAC-MS. If a transcript had more than one edit site, the site with the greatest % change was used. For CYFIP2 and CAPDS, values were obtained from Sanger sequencing.

FIGS. 6A-6I. Nuclear accumulation of A-to-I edited RNA in synucleinopathy patient iPSn and brain. A) Quantification of Inosine-containing poly (A) mRNA isolated from cytosol (Cyt) or nucleus (Nuc) of PD or corrected iPSn (n=5 culture wells, left graph). Equal amounts of mRNA per line (30 ng) were applied to membranes as determined by absorbance at 260 nm, and A260:280 ratios (right graph). B) Immunofluorescence analysis of PD (A53T) iPSn and isogenic corrected controls revealing inosine puncta that accumulates and colocalizes with NONO in the nucleus of patient cultures. RNAse treatment of fixed cultures reduces the inosine signal, indicating that it is derived from A-I edited RNA. Middle graph, Quantification of inosine puncta in the nucleus. Values were averaged and plotted from each culture well (n=4). C) Representative RNA-FISH confocal image of polyA+ RNA with oligo-dt probes showing increased nuclear accumulation in A53T patient compared to isogenic corrected (corr) iPSn. Nuclei are stained with DAPI. Scale bar, 10 um. Right, Quantification of percentage of cells with nuclear polyA+ foci in patient A53T iPSn compared to corr. Each datapoint represents an individual culture well (n=3). D) RNA-FISH of polyA+ mRNA in human frontal cortex tissue of healthy control and Dementia with Lewy Bodies (DLB) patients with immunohistochemistry for NONO (red) and neuronal marker NeuN (white). The white arrows indicate colocalization of NONO with polyA+ RNA foci. DAPI (blue) stains nucleus. Secondary antibody alone was used as a negative control. Scale bar, 10 um. Middle graph, quantification of Pearson's coefficient indicating the colocalization of individual nuclei from 3-4 different brains. Each data point indicates a NeuN+ nuclei taken from n=3 control and 4 DLB patients. Right graph, Pearson values were averaged and plotted from each individual brain. E) Quantification of nuclear mRNA levels of edited transcripts by RT-PCR relative to cytoplasmic mRNA levels. Each fraction was normalized to total mRNA levels of the respective transcript. F) Quantification of control mRNA transcripts that are not edited. G) FISH analysis of CADPS and CYFIP2 in fixed PD and isogenic corrected lines. PD lines showed an accumulation of mRNA inclusions of 1-2 um in diameter (white arrows). Right, Quantification of nuclear mRNA puncta normalized to nuclei (DAPI), where each color plot represents an individual culture well, and quantification of % of cells containing large puncta (1-2 um in diameter) where one plot represents an average value from an individual culture well. H) Immunofluorescence analysis of SFPQ (green) followed by FISH to detect PRKAR2A mRNA (red). Quantification of nuclear: cytoplasmic ratio of PRKAR2A is shown to the right (n=3 culture wells). I) Western blot validation of axon/synaptic proteins that are reduced in PD iPSn. GAPDH and actin are non-edited controls and indicate loading along with CBB. Right, Quantification of western blot data normalized to CBB. The values are represented as mean±SEM, *p<0.05; **p<0.01 and ****p<0.0001, using student's unpaired t-test.

FIGS. 7A-7K. Reducing A-to-I editing reverses nuclear retention of mRNA and restores expression of axon/synaptic proteins. A) Quantification of inosine-containing mRNA in A53T iPSn by anti-inosine dot blot of poly (A) purified mRNA, treated with 200 nM 8-aza-adenosine (8-aza) for 30 days. B) Confocal analysis of polyA+RNA-FISH of A53T patient iPSn cultures treated as in panel A. Nuclei are stained with DAPI. Scale bar, 10 um. Right, quantification of percentage polyA+ foci containing DAPI. Each datapoint represents the average from n=3 culture wells. C) Western blot analysis of ADAR3 after lenti-viral infection. Lenti-GFP was used as a control (n=4 culture wells). D) Quantification of polyA+ foci after GFP or ADAR3 expression in A53T PD iPSn, analyzed as in B. E) Quantification of nuclear mRNA levels of edited axonal and synaptic transcripts by RT-PCR relative to cytoplasmic levels in A53T iPSn cultures treated as in panel A. Actin was used as non-edited control (n=4-5 culture wells). F) Western blot analysis of corresponding axonal and synaptic proteins treated as in panel A. CBB, GAPDH, and βiii-Tubulin are loading controls. G) Quantification of panel F, normalized to CBB (n=8-9 culture wells). H) Western blot analysis of corresponding axonal and synaptic proteins treated as in panel C. I) Top, eGFP plasmid used showing the region of PRKAR2A 3′UTR and the % edit increase found in PD iPSn by RNA-seq. Bottom, Image of fixed HEK cells that were co-transfected with eGFP-PRK-3′UTR and either vector control or ADAR1 E713Q hyper-active mutant. DAPI indicates nuclear localization and white signal indicates GFP mRNA detected by FISH. % of total cells with nuclear GFP signal was quantified on the top graph and total GFP signal normalized to DAPI was quantified on the bottom (n=3 individual culture wells). J) Western blot analysis of PD iPSn (A53T) or isogenic controls (Corr) transduced with eGFP plasmids fused to the 3′UTR of PRKAR2A or without a 3′UTR (eGFP control). Quantification represents values from 3-4 culture wells. Transduced cultures were treated with 8-aza as in panel A and analyzed by GFP western blot. K) Immunostaining analysis of fixed cultures from isogenic controls (Corr) or PD iPSn (A53T) treated with veh or 8-aza as in A, using PSD-95 (post-synaptic marker) and synaptophysin (pre-synaptic marker). b-III-Tubulin and DAPI were used to visualize neuronal density. Images were selected based on approximately equal levels of neurites, estimated by b-iii-Tubulin. Below, Quantification of PSD-95/synaptophysin colocalized puncta. Colocalized signals of detectable puncta (both faint and intense) were quantified to estimate pre- and post-synaptic overlapping signals. Each plot represents an average quantification from an individual culture well (n=4 wells). See FIG. 15 for individual channel images. All values are the mean±SEM, *p<0.05; **p<0.01, ***p<0.001 and ****p<0.0001, using student's unpaired t-test (A-I), and ANOVA with Tukey's post-hoc test for J, K.

FIGS. 8A-8M. A-to-I edited RNAs trigger pathological protein aggregation. A) Sequential extraction/western blot analysis of A53T iPSn treated with 8-aza as in FIG. 7A. Quantification is shown on the right, normalized to CBB (Coomassie brilliant blue). biii-Tubulin and CBB are loading controls. B) Western blot analysis of ADAR3 levels in 8-aza treated A53T iPSn cultures (n=5). CBB (Coomassie blue), GAPDH and βiii-Tubulin are loading controls. Right, quantification normalized to CBB. C) A53T iPSn were infected with lenti-virus to express GFP or ADAR3 (MOI 3, dpi 14) and analyzed by sequential extraction/western blot for NONO and SFPQ. The dotted lines indicate cropped out irrelevant lanes, but samples were run on the same blot. D) Healthy control cultures were infected to overexpress hyperactive forms of ADAR1 and ADAR2 and analyzed sequential extraction/western blot to assess SFPQ and NONO solubility. Lysates were treated with either RNAse A or Endo V in vitro to remove total or inosine-containing RNA, respectively. CBB (Coomassie brilliant blue) was used as a loading control. The dotted lines indicate cropped out lanes, but samples were run on the same blot. Right, quantification of western blots. E) Native PAGE of RNA oligonucleotides (oligos) encompassing edit site 2 of PRKAR2A 3′UTR, detected by SYBR green II. Right, measure of oligo migration according to ssRNA molecular weight (MW) standards, in nucleotides (nt). M, Marker. F) Denaturing PAGE of RNA oligos as in E. Quantification to the right represents difference in migration between non-edited or A-to-I edited oligos, where each plot represents an individual gel lane replicate from 3 experimental setups. G) Denaturing PAGE analysis of RNA oligos that were digested with RNAse T1 or RNAse I. The dotted line indicates irrelevant lanes that were cropped out but were run on the same blot. H) 100 nM RNA FAM-labeled oligos were mixed with SFPQ at the indicated concentrations and analyzed by native TBE PAGE. FAM fluorescence of the bound oligo was quantified in the graphs below. Arrows indicate RNA oligo bound to SFPQ that is likely at 3 different structural states. Quantification in the bar graph represents 3 different experimental setups. I) Sedimentation/western blot analysis of 10 uM SFPQ mixed with or without RNA oligos at 1 uM. Quantification shown below represents 4 individual experimental setups. S, supernatant; P, pellet. J) Sedimentation analysis of RNA oligos from the same samples shown in panel I, mixed with or without recombinant SFPQ, analyzed by denaturing PAGE and detected by SYBR green II. Quantification shown below represents 4 individual experimental setups. K) RT-qPCR to measure RNA levels of NEAT1_2 in corrected or A53T iPSn. L) RT-qPCR to measure RNA levels of NEAT1_2 in post-mortem brain tissues from healthy age-matched controls or DLB patients. M) RT-qPCR to measure RNA levels of NEAT1_2 in A53T iPSn treated with the A-to-I RNA editing inhibitor, 8-aza. Values are the mean, +/− SEM. *p<0.05,**p<0.01, ***p<0.001, ****p<0.001 Student's unpaired t-test for panel A-C, F, and H, and ANOVA with Tukey's post-hoc test was used for D, I, and J.

FIGS. 9A-9N. Specificity and analysis of physicochemical properties of aggregated proteins in A53T iPSn. A) Protein quantification of essential paraspeckle proteins quantified by SILAC-MS in Corr or A53T iPSn, shown as fold control compared to Corr (n=4 for each time point). B) Analysis of LLR (Log-likelihood ratio) score, and indication of the presence of prion-like domains, plotted against the change in solubility shows a significant correlation. Only significantly changed proteins were used (white plots from FIG. 1A). C, D) Analysis of supersaturation values of proteins in the unfolded or folded state plotted against solubility. E-H) Analysis of molecular weight (MW), charge, isoelectric point (pI), or propensity to form beta-pleated sheet with solubility showed no significant correlations. I) SILAC labeled control iPSn were treated with 100 uM Leupeptin to inhibit lysosomal proteolytic activity for one month, then analyzed by sequential extraction/SILAC-MS as in FIG. 1 (n=4, *p<0.05). J) Control iPSn treated with 50 uM L-DOPA were analyzed by sequential extraction/western blot. Right, quantification of NONO/SFPQ normalized to CBB (n=4). The right graph indicates the level of oxidized catechols detected in intracellular lysates of A53T iPSn, as assessed by near-infrared fluorescence (nIRF) assay previously described (n=4) (Mazzulli et al, Anal. Chem, 2016). Values are the mean, +/− SEM. *p<0.05, Student's t-test. For each assay, each plot represents an individual culture well. K) Sequential extraction/western blot of FUS and PSPC1 from d60 and d90 A53T iPSn cultures, validating the SILAC-MS data from FIG. 1A, B (n=4 culture wells). L) Quantification of panel K. M) Quantification of mRNA from day 90 A53T and isogenic control midbrain neuronal cultures by RT-PCR normalized to β-actin (n=3). N) d90 A53T iPSn showing accumulation of insoluble α-syn(n=3). Values are the mean +/− SEM, *p<0.05, ****p<0.001, Student's t-test was used for all comparisons.

FIGS. 10A-10E. Pathological analysis of insoluble proteins in SNCA 3× iPSn and DLB brain tissues. A) Western blot analysis showing soluble and insoluble fractions of RNA-binding proteins and α-synin d60 SNCA 3× patient, isogenic control and healthy control iPSn (n=3). Below, quantification represented as insolubility fold change. Right, Western blot analysis of RNA-binding proteins and accumulation of insoluble α-synin d90 SNCA 3× patient and isogenic control iPSn (n=3). Values are the mean, +/− SEM. *p<0.05, Student's t-test. B) Sequential extraction/western blot analysis of post-mortem brain samples (frontal cortex) obtained from the Mayo Clinic Brain Bank (Jacksonville FL). Samples were sequentially extracted as described in FIG. 2D (high-salt soluble, 1% Sarkosyl insoluble (pellet resuspended in PBS)). Quantification of the blot is shown in FIG. 2D. Each plot represents an individual brain sample C) Sequential extraction/western blot analysis of post-mortem brain samples (frontal cortex) obtained from the Northwestern University Brain bank. D) Quantification of insolubility normalized to total protein (CBB) is shown below (n=4 controls, n=9 DLB; male, female), E) Grayscale image and representative threshold processing of nuclei from FIG. 2E, showing quantification of nuclear NONO puncta using Airlocalize software. Each plot (n) represents an individual puncta from n=1 control and n=1 DLB brain. Values are the mean, +/− SEM. *p<0.05, ****p<0.001 Student's unpaired t-test for panel D and E; ANOVA with Tukey's post-hoc test was used for A.

FIGS. 11A-11F. Colocalization of α-syn with NONO/SFPQ in the nucleus of PD patient iPSn and DLB post-mortem brain. A) Representative confocal stacks of d90 PD (A53T) orisogenic control lines using syn211 (C-term, AA 120-125), NONO (A11), and polyclonal SFPQ antibodies. The boxed in nuclei from A53T iPSn is shown in FIG. 3B, representing a 1 um confocal section to demonstrate colocalization in the nuclear focal plane. The images were quantified in FIG. 3B, right. B) Representative immunostaining images of α-syn (C20 antibody, C-term) colocalizing with NONO (A11) mainly in a diffuse pattern in the nucleus. Pearson's correlation indicates stronger colocalization in PD cultures. C) Examples of punctated α-syn/NONO staining in the nucleus of PD iPSn using α-syn antibody C20 occurred in approximately 20% of cells. Each plot represents an individual culture well. D) Immunofluorescence analysis using syn505 (N-term, AA 2-4) that preferentially detects pathogenic conformations, NONO (A11), and SFPQ shows colocalization and accumulation in the nucleus of PD iPSn. SNCA knock-out (KO) iPSn were used to assess antibody specificity of syn505. E) Immunohistological analysis of control and dementia with Lewy body (DLB) brains (two individual brains shown as representative examples) showing colocalization of α-syn(C20) with NONO (A11 direct conjugate antibody) and SFPQ (clone D8). Arrows indicate examples of colocalized nuclear inclusions. F) NONO and SFPQ do not colocalize with α-synin Lewy bodies or Lewy neurites. A-syn was detected with syn211, NONO with direct conjugate A11 mouse antibody, and SFPQ with a polyclonal rabbit antibody (see methods for details). Values are the mean, +/− SEM. *p<0.05, **p<0.01 Student's unpaired t-test was used for panels B and C.

FIGS. 12A-12E. Reduced ADAR3 mRNA by SFPQ knock-down and assessment of RNA editing in iPSn and DLB brain. A) Western Blot analysis showing siRNA-mediated knockdown of SFPQ in iPSC-neurons from healthy controls after 5 days. Cultures were transfected with scrambled (scrb) siRNA as control. CBB (coomassie blue), GAPDH, and βiii-Tubulin are loading controls. B) mRNA expression levels of SFPQ and its transcriptional target ADAR3 in healthy neuronal cultures measured by q-RT-PCR. mRNA levels were normalized to β-actin. Quantification represents mean±SEM, *p<0.05; ***p<0.001, using student's unpaired t-test. C) mRNA expression levels of ADAR1 assessed by q-RT-PCR in d60 A53T and isogenic corrected iPSn. (n=4 culture wells). D) Western blot analysis of ADAR1 and 2 in A53T and isogenic corrected lines at d60 (n=4-8 culture wells). E) Representative sequencing chromatograms of CYFIP2 edit region from control and DLB patients. The genomic DNA (gDNA) is shown on the top and the corresponding complementary sequence amplified from RNA (cDNA) appears on the bottom for each patient. The red arrow indicates the edit site location. Patient IDs are shown in top left box. Quantification is shown in FIG. 4 I-K.

FIGS. 13A-13D. Network analysis of proteins with reduced expression in A53T iPSn. A) Gene enrichment analysis of D60 cultures was done using Cellular Component Gene Ontology database. B) Select genes involved in axonal or synaptic function that are both established RNA A-to-I editing targets (assessed by accessing the RADAR database, Ramaswami and Li, Nucleic Acids Res. 2014, accessed between 2018-2020) and have corresponding reduced protein levels in PD-iPSn by SILAC-MS. C) Network analysis of reduced proteins in A53T iPSn reveals a significant overrepresentation in neuronal development, axon guidance, and synaptic function. Day 60 and 90 proteomic data from SILAC-MS quantification of proteins with decreased total abundance in A53T iPSn were analyzed by to reveal enrichment in protein networks using the Reactome database. Each node represents and individual network and edges reveal network interactions. Node color represents p-value, while node size indicates the number of corresponding protein-encoding genes per network. D) Day 60 Reactome analysis of decreased proteins in A53T iPSn, showing individual networks and corresponding p-values. This data was used to prioritize the study of axon guidance and synaptic proteins.

FIGS. 14A-14F. Specificity assessment of anti-inosine antibody and validation of decreased axon/synaptic proteins in patient iPSn and DLB brain. A) Equal amounts of purified 30 base-pair RNA oligonucleotide from the PRKAR2A 3′UTR containing either non-edited sequence (adenosine, guanosine, cytidine, and uridine (top)), or inosine in the place of adenosine (bottom) were applied to membranes at the indicated quantities in nanograms (ng) and probed with anti-inosine antibodies to assess the specificity of the inosine antibody. The sequences of the oligos are shown in FIG. 16C. B) Total mRNA expression levels of edited targets measured by RT-PCR normalized to β-actin (n=6-7 culture wells). C) Total abundance quantification of select axonal and synaptic proteins measured by SILAC-MS at day 60, quantified as fold change of Corr. D, E) Western blot analysis of the soluble fractions of DLB brain (frontal cortex). F) Quantification of the blots from D and E. Each plot represents a measurement from an individual patient's brain. Values are the mean±/− SEM, *p<0.05, **p<0.01, ****p<0.001 using Student's unpaired t-test.

FIGS. 15A-15G. Reduction of A-to-I editing by 8-azaadenosine and rescue of neurotoxicity in A53T iPSn. A) HEK cells were transfected with a fluorescent RNA editing reporter expressing an A-I editing linker flanked by RFP and GFP. Reduction of editing activity is observed by expression of both GFP and RFP in yellow cells. Right, quantification of GFP/RFP ratios (n=11 fields of view from 3 culture wells). B) Sanger sequencing of genomic and cDNA reveals that 8-aza treatment inhibits the editing of CYFIP2 transcript in A53T iPSn (n=4). Each plot represents a measure from an individual culture well. C) PolyA mRNA was isolated from 8-aza treated A53T iPSn then analyzed by UPLC-MS to detect RNA nucleotides for specificity analysis of the drug. D) The total mRNA levels of edited axonal and synaptic transcripts quantified by RT-PCR normalized to β-actin (n=3). E) Immunostaining analysis of fixed cultures from isogenic controls (Corr) or PD iPSn (A53T) treated with veh or 8-aza as in A, using PSD-95 (post-synaptic marker) and synaptophysin (pre-synaptic marker). b-III-Tubulin and DAPI were used to visualize neuronal density. Images are of the separate channels of the merged image shown in FIG. 7K. F) Western blot analysis of synaptophysin shows reduced levels in A53T iPSn that are rescued by 8-aza treatment, supporting the immunofluorescence data in panel E. G) Neuron viability was assessed by measuring the levels of LDH released into the media of A53T iPSn at day 120 treated with veh or 200 nM 8-aza for 30 days (n=5 culture wells). Each plot represents a measure from an individual culture well. For each assay, each plot represents a measure from an individual culture well.

FIGS. 16A-16E. A-to-I conversion on PRKAR2A 3′UTR induces structural disorder and promotes increased binding and aggregation of SFPQ. A) Healthy control cultures were infected to overexpress hyperactive forms of ADAR1 and ADAR2 and analyzed by western blot to assess the amount of overexpression. Lenti-RFP was expressed at equal titers as a control. Note that the functional form of ADAR1 naturally occurs in the insoluble fraction under our extraction conditions (linked to FIG. 8D). B) Predicted RNA secondary structure by RNAfold of the 1 Kb 3′UTR of PRKAR2A encompassing the editing enriched region that is hyper-edited in PD iPSn. Right, Structural analysis of the AluJr region where edit site 2 is changed from adenosine to inosine causing a predicted disruption in the double-stranded stem structure. Changing adenosine to inosine in sites 1, 3, 4, and 5, cause no predicted effect on secondary RNA structure, possibly because some of these adenosines do not participate in base-pairing and are present in bulges. Site 2 adenosine (position 94 from the start site) normally participates in an A-U pairing, and conversion to inosine forms an unstable I-U pair, resulting in structural collapse. Non-edited AluJr is SEQ ID NO: 1; A-to-I edited AluJr at site 2 (94 bp) is SEQ ID NO: 2. The bold/underlined adenosine (a) is an important site of A-to-I RNA editing that is increased in PD neurons that results in RNA structure changes. C) RNA oligonucleotides containing edit site 2 that were used for analysis. RNAfold predicts converting adenosines to inosines disrupts the stem structure, similar to the effect on the full length 3′UTR. This is the RNA oligo that was used in FIG. 8 E-J. Potential SFPQ binding sites are outlined in red. Un-edited PRK 3′ UTR oligo 1 is SEQ ID NO: 3; A-to-I edited PRK 3′UTR oligo 1 is SEQ ID NO: 4. D) Native polyacrylamide gel electrophoresis/western blot of SFPQ species mixed with PRK 3′UTR oligos indicates that inosine-containing oligonucleotides promote high molecular weight (HMW) SFPQ species. The dotted line indicates irrelevant lanes that were cropped out but were run on the same blot. Quantification is on the right, n=3 separate experimental setups. Values are the mean±/− SEM. *p<0.05, **p<0.01, Student's t-test (panel A), or ANOVA with Tukey's post-hoc test (panel D). E) Summary of the pathogenic role of increased RNA A-to-I editing and NONO/SFPQ inclusions in PD/DLB. The pathogenic process is initiated by interaction of soluble α-syn oligomers with NONO/SFPQ, inducing the conversion into insoluble inclusions in the nucleus. This occurs prior to the formation of insoluble α-syn or Lewy inclusions. Elevations in A-to-I editing are triggered by ADAR3 depletion via sequestration of soluble, functional SFPQ into inclusions bodies. Edited mRNAs then have a dual pathogenic function by potentiating the aggregation of nuclear inclusions (red arrow) and retention in the nucleus prevents them from being translated into protein. The reduction of vital axon, synaptic, and mitochondrial proteins results in neurodegeneration.

FIG. 17. Symptomatic Transgenic mice expressing human A53T α-synuclein do not exhibit aggregation of NONO, SFPQ, FUS, or PSPC1. Spinal cord from symptomatic transgenic mice expressing human A53T α-synuclein (line M83 driven by PrP¹²⁷) aged between 11 and 16 months of age were analyzed by sequential extraction/western blot analysis. The spinal cord was chosen since this region has the most severe synucleinopathy. CBB, Coomassie brilliant blue was used as a loading control. Each gel lane represents a sample from a different mouse brain. Age-matched non-transgenic (nTg) mice of the same background were used as a control.

FIG. 18. Summary of the new pathology and disease mechanisms, and methods to restore neuronal health. I) The inventors describe a new mechanism showing that A-to-I edited RNAs that accumulate in the nucleus are critical for promoting NONO/SFPQ aggregation and translational shut down. Once NONO/SFPQ are sequestered into insoluble, non-functional aggregates, they cannot perform their normal functions. One critical function of SFPQ is to act as a transcriptional co-activator and turn on the transcription of ADAR3, a brain-specific RNA inhibitor. Data demonstrates that A-to-I editing unwinds dsRNAs and allows them to bind with greater affinity to SFPQ. This binding process dramatically promotes its aggregation into insoluble inclusions. This produces a self-propagating pathogenic cycle of disease that culminates in reduced nuclear export and translation of essential mRNAs required for synaptic function. II) Small molecule inhibitor of ADARs 1 and 2 called 8-aza-adenosine, as well as overexpression of ADAR3 to reduce RNA editing can rescue this process. These treatments allow double stranded IRAlus to remain structured, and therefore they interact less with SFPQ and prevent its aggregation (Bottom Panel II, Arrows 1 and 2). Once solubilized, NONO and SFPQ become functional once again and SFPQ can resume its normal activity as a transcriptional co-activator of ADAR3 and restore its expression (Panel II, Arrow 3). As a consequence of reduced editing, the mRNAs are no longer sequestered into protein aggregates and are therefore free to be exported to the cytoplasm where translation occurs (Panel II, Arrow 4). This in turn restores expression of axon and synaptic proteins, and reverses synaptic loss in PD and DLB (Panel II, Arrow 5).

FIGS. 19A-19B. Selective reduction of NEAT1_2 isoform reduces pathological SFPQ aggregation in PD midbrain neurons. A) A53T iPSn were transfected with control or NEAT1_2 targeted gapmers (ASO) followed by analysis of NEAT1_2 transcript levels by Q-RT-PCR (4 days post-transfection, 300 nM). NEAT1_2 transcript levels were normalized to beta-actin mRNA (ACTB). B) Sequential extraction/Western blot analysis for SFPQ of PD A53T iPSC-midbrain neurons to measure insoluble levels of SFPQ after NEAT1_2 knock-down (KD). KD was achieved by CRISPR-Cas9 targeting of NEAT1_2 at the 3′ end of the transcript and compared to control A53T iPSn that express elevated levels of NEAT1_2. Values are the mean, +/− SEM, n=3-4 biological replicates, ns=not significant, **p<0.01.

### DETAILED DESCRIPTION

The present disclosure provides methods for treating neurodegenerative diseases including dissolving and inhibiting pathological aggregates and restoring neuronal synaptic function. The inventors demonstrate that pathological aggregates in the nucleus of patients with synucleinopathies comprise RNA binding proteins (including NONO and SFPQ), the long non-coding RNA called NEAT1_2, and mRNA that trigger Adenosine (A) to Inosine (I) RNA editing. An increase in A to I RNA editing promotes additional aggregation of synaptic, axonal and mitochondrial transcripts. The inventors found that inhibiting A to I RNA editing in these cells dissolves pathological aggregates and restores neuronal synaptic health.

In one aspect, the present disclosure provides methods of treating neurodegenerative diseases. A neurodegenerative disease is caused by the progressive loss of structure or function of neurons, in the process known as neurodegeneration. Such neuronal damage may ultimately involve cell death. Neurodegenerative diseases include but are not limited to amyotrophic lateral sclerosis, multiple sclerosis, Parkinson's disease, Alzheimer's disease, Huntington's disease, dementia with Lewy bodies, multiple system atrophy, tauopathies, and prion diseases. Some neurodegenerative diseases are classified as proteinopathies as they are associated with the aggregation of misfolded proteins. Protein toxicity is one of the key mechanisms of many neurodegenerative diseases. Proteins that have been associated with aggregates include, but are not limited to alpha-synuclein, tau, amyloid beta and prion. Neurodegenerative diseases characterized by the abnormal accumulation of aggregates of alpha-synuclein are called synucleinopathies. Alpha-synuclein can aggregate to form insoluble fibrils in pathological conditions characterized by Lewy bodies, such as Parkinson's disease, dementia with Lewy bodies, and multiple system atrophy. Alpha-synuclein is the primary structural component of Lewy body fibrils. In addition, an alpha-synuclein fragment, known as the non-Abeta component (NAC), is found in amyloid plaques in Alzheimer's disease.

The methods of treating neurodegenerative disease comprise inhibiting Adenosine (A) to Inosine (I) RNA editing. Adenosine-to-Inosine (A to I) RNA editing is a post-transcriptional modification that converts adenosines to inosines in both coding and noncoding RNA transcripts. It is catalyzed by ADAR (adenosine deaminase acting on RNA) enzymes, which exist throughout the body but are most prevalent in the central nervous system. Inosines exhibit properties that are most similar to those of guanosines. As a result, ADAR-mediated editing can post-transcriptionally alter codons, introduce or remove splice sites, or affect the base pairing of the RNA molecule with itself or with other RNAs.

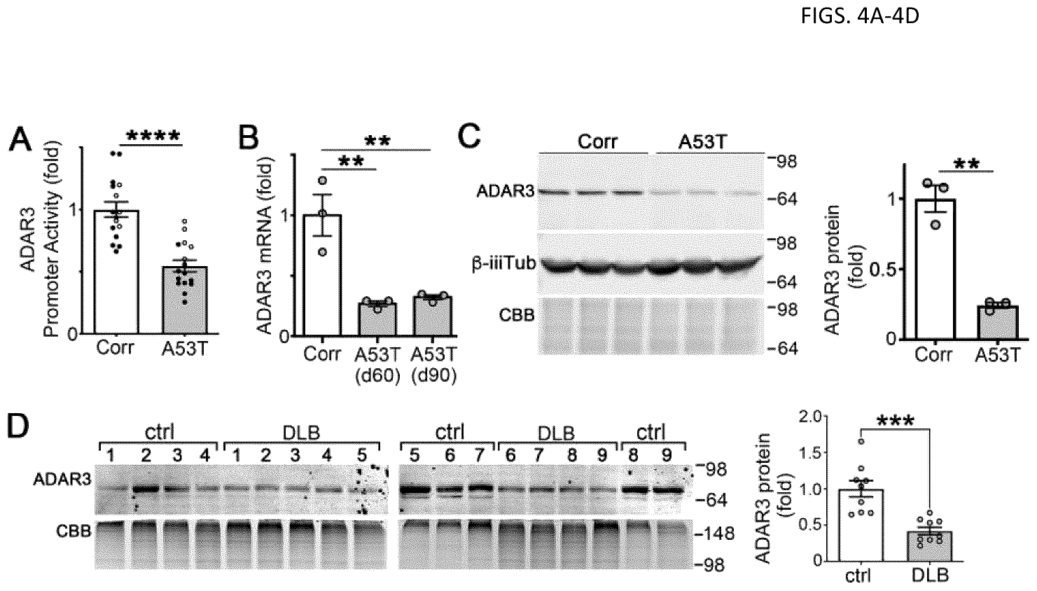

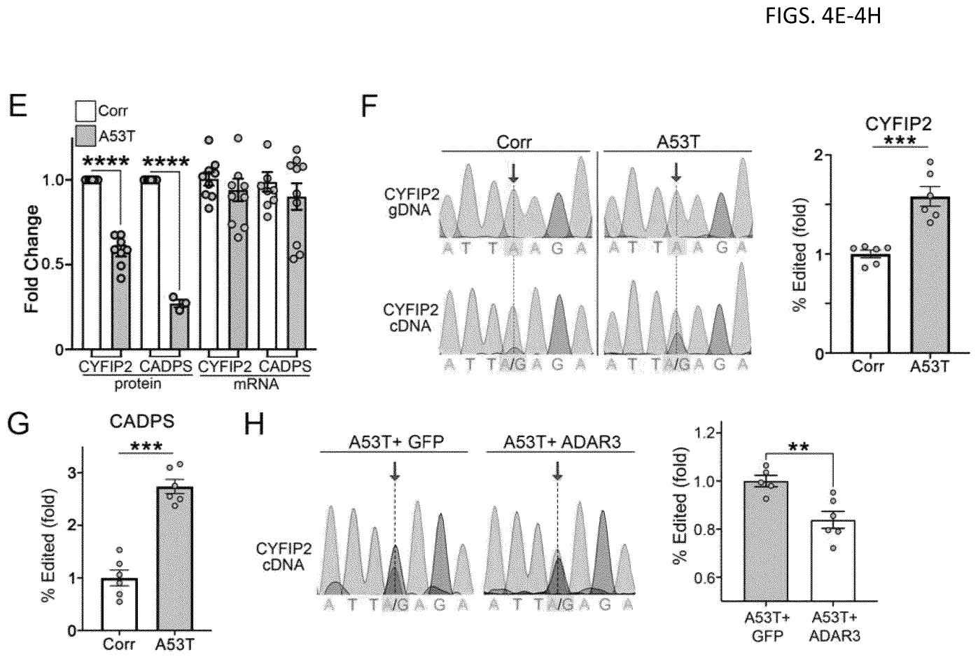

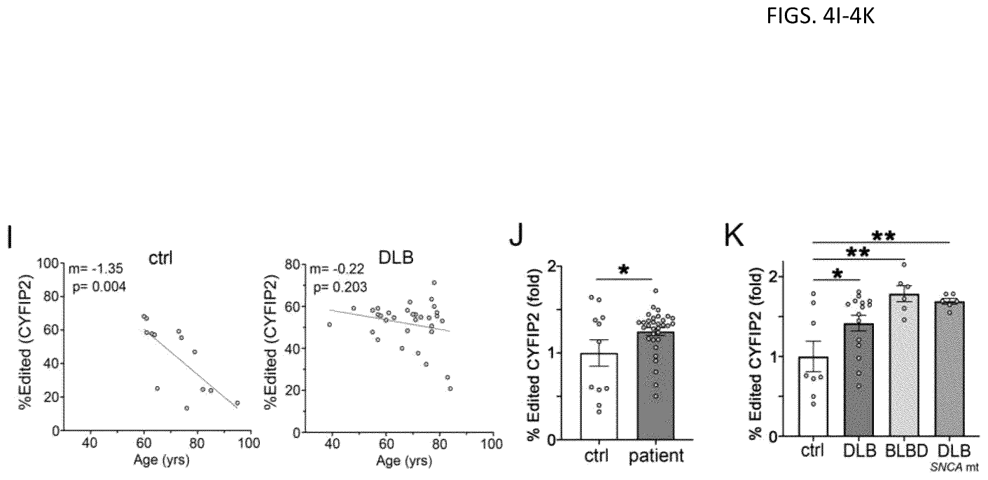

Three primary members of the ADAR family have been identified in humans: ADAR1, ADAR2 and ADAR3. ADAR1 and ADAR2 are present in many tissues, whereas ADAR3 is specifically expressed in brain tissues and is believed to be catalytically inactive. ADAR3 binds to dsRNA sequences in competition with the enzymatically active ADARs 1 and 2 and inhibits A-to-I editing. By inhibiting A-to-I editing, overexpression of ADAR3 promotes dsRNA structure, while its depletion can reduce dsRNA structure, consistent with the effect of inosine on destabilizing dsRNA. ADARs contain a conserved deaminase domain that mediates A-to-I editing, as well as variable double-stranded RNA-binding domains that are required for substrate specificity and binding. Changes in ADAR activity, caused by mutations or changes in expression, have been associated with a wide range of human diseases, including cancer, neurological disorders, metabolic diseases, viral infections and autoimmune disorders. Specifically, increased A-to-I RNA editing in Parkinson's disease (PD) brain has been independently confirmed in other labs supporting our findings from DLB brain (FIGS. 4I-K) and strengthening the rationale to reduce A-to-I editing as a treatment for synucleinopathies (D'Sa et al., Astrocytic RNA editing regulates the host immune response to alpha-synuclein. Sci Adv. 2025 Apr. 11; 11(15):eadp8504.).

NonPOU Domain-Containing Octamer-Binding Protein, (NONO) and Splicing Factor, Proline- and Glutamine-Rich, (SFPQ) participate in transcriptional and posttranscriptional regulation as well as contribute to paraspeckle subnuclear body organization. NONO and SFPQ are members of the Drosophila Behavior/Human Splicing (DBHS) family of RNA/DNA binding cofactors and participate in a range of cellular processes including binding DNA and RNA via highly conserved RNA-binding domains. NONO/SFPQ contain low complexity prion-like domains that allow for liquid-phase transitions into functional aggregates that preferentially and directly bind to inosine-containing mRNAs. Described herein, the inventors found that nuclear aggregates comprised mainly NONO, SFPQ, and A-to-I editing mRNA accumulate in the nucleus of patients with synucleinopathies and shut down the production of essential proteins that encode axon, synaptic, and mitochondrial proteins.

NONO and SFPQ have also been linked to other neurodegenerative diseases. Genetic variants of SFPQ are associated with motor neuron disease and synaptic abnormalities, and SFPQ aggregates occur in amyotrophic lateral sclerosis^{57,58}. Loss of NONO in humans causes a severe neurodevelopmental disorder characterized by intellectual disability and loss of synapses, establishing its significance in maintaining neuronal health⁵⁹. Therefore, the methods provided in this disclosure may also apply to neurodegenerative and neurodevelopmental diseases described above that demonstrate specific loss of function in either SFPQ and/or NONO proteins.

Nuclear aggregates of NONO and SFPQ are stimulated by expression of the long non-coding RNA called nuclear-enriched abundant transcript 1_2 (nuclear paraspeckle assemble transcript or NEAT1_2), which forms a scaffold for NONO and SFPQ proteins to interact and aggregate. Overexpression of NEAT1_2 can occur specifically from mitochondrial stress (Wang Y. et al, Genome-wide screening of NEAT1 regulators reveals cross-regulation between paraspeckles and mitochondria. Nat Cell Biol. 2018 October; 20(10):1145-1158), which is prevalent in Parkinson's disease and related synucleinoapthies. NEAT1_2 overexpression has been documented in many neurodegenerative diseases including Parkinson's disease, Lewy Body dementia, Amyotrophic Lateral Sclerosis, Frontotemporal dementia, Huntington's disease, and Alzheimer's disease (An H. et al, NEAT1 and paraspeckles in neurodegenerative diseases: A missing Inc found?Noncoding RNA Res. 2018 Nov. 15; 3(4):243-252). Given that these diseases have documented pathology that is similar to what the inventors uncovered in synucleinoapthies, including NEAT1_2 accumulation, NONO, and SFPQ aggregation, the methods described here may be applied as therapies to treat these other diseases. Specifically, reduction of the NEAT1_2 isoform and/or inhibition of RNA editing in these diseases is expected to dissolve pathology and restore neuronal health.

The A to I inhibitor may comprise a small-molecule drug, an oligonucleotide, or an antibody or antibody fragment. An inhibitor may be a competitive inhibitor or a non-competitive inhibitor, may be reversible or irreversible. The A to I inhibitor may inhibit one or more ADAR1 and ADAR2. The A to I inhibitor may be an ADAR3 activator. ADAR 1 and ADAR2 inhibitor may inhibit ADAR1 and ADAR2 expression or activity. ADAR3 activators may increase ADAR3 expression or activity. Oligonucleotide inhibitors include interfering RNAs. RNA interference is a biological process in which RNA molecules are involved in sequence-specific suppression of gene expression by double-stranded RNA, through translational or transcriptional repression. Without limitation examples of interfering RNA include microRNA, small interfering RNA (siRNA), short/small hairpin RNA (shRNA), and piwi-interacting RNA (piRNA).

The A to I inhibitor may comprise a small-molecule drug incorporated into an oligonucleotide structure. For example, a small-molecule drug/oligo inhibitor of ADAR1 and ADAR2 may be 8-Azanebularine-Modified RNA Duplexes (Mendoza et al., Selective Inhibition of ADAR1 Using 8-Azanebularine-Modified RNA Duplexes. Biochemistry. 2023 Apr. 18; 62(8):1376-1387). ADAR3 can be increased by any means known in the art, for example by overexpression using a lentiviral vector as shown in Example 1. In exemplary embodiments, the ADAR1 inhibitor is 8-Aza-Adenosine (8-aza) (CAS No. 10299-44-2). In preferred embodiments, the 8-aza is administered at a sub-toxic dose. A sub-toxic dose is a dose that does not induce cell death. The 8-aza may be administered at less than about 1 μM. The 8-aza may be administered at between about 0.05 μM and about 1 μM, and any dose or range in between. The 8-aza may be administered at about 200 nM.

The A to I inhibitor may comprise an oligonucleotide inhibitor, such as an antisense oligonucleotide, an interfering RNA, or a guide RNA.

In some embodiments, the inhibitor comprises a NEAT1_2 inhibitor. The NEAT1_2 inhibitor may reduce or inhibit at least one of NEAT1_2 expression and activity. The NEAT1_2 inhibitor may comprise a small-molecule drug, an oligonucleotide, or an antibody or antibody fragment. In exemplary embodiments, the NEAT1_2 inhibitor reduces NEAT1_2 expression. In exemplary embodiments, the NEAT1_2 inhibitor is 8-aza or an anti-sense oligonucleotide such as SEQ ID NO: 5.

As used herein “inhibit” means to reduce, attenuate, stop, make less likely or prevent as dependent on context. For example, an inhibitor of A to I RNA editing may reduce, prevent, or decrease A to I editing. A to I editing can be measured by any means know in the art including but not limited to sequencing, and High-performance liquid chromatography-mass spectrometry (HPLC-MS). An inhibitor may directly or indirectly inhibit expression or activity of a molecule. As used herein, the term “decrease” or the related terms “decreased,” “reduce” or “reduced” refers to a statistically significant decrease. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero).

The methods provided herein may decrease pathological aggregates in the nucleus, particularly of cells within the brain. Pathological aggregation refers to the accumulation of aggregated proteins, or protein oligomers in the form of intracellular inclusions that causes cellular toxicity, injury and or cell death. Pathological aggregates may comprise aggregated proteins and aggregated RNA.

In another aspect, a method of preventing or dissolving pathological aggregates in the central nervous system is provided, the method comprising inhibiting adenosine (A) to inosine (I) RNA editing to a cell or subject in need. As used herein, “preventing” or “dissolving” as it refers to pathological aggregates means to reduce the accumulation of aggregates, decrease the size of aggregates, decrease or slow the accumulation of aggregated protein or RNA. In embodiments, new aggregates are prevented from forming. In other embodiments existing aggregates decrease in size or frequency. The pathological aggregates may comprise at least one of NonPOU Domain-Containing Octamer-Binding Protein (NONO) and Splicing Factor, Proline- and Glutamine-Rich (SFPQ).

The A to I inhibitor may comprise a small-molecule drug, an oligonucleotide, or an antibody or antibody fragment. An inhibitor may be a competitive inhibitor or a non-competitive inhibitor, may be reversible or irreversible. The A to I inhibitor may inhibit one or more ADAR1 and ADAR2. The A to I inhibitor may be an ADAR3 activator. ADAR 1 and ADAR2 inhibitor may inhibit ADAR1 and ADAR2 expression or activity. ADAR3 activators may increase ADAR3 expression or activity. ADAR3 can be increased by any means known in the art, for example by overexpression as shown in Example 1.

A “subject in need thereof” as utilized herein may refer to a subject in need of treatment for a neurodegenerative disease. A subject in need thereof may include a subject having, or suspected of having Alzheimer's disease, amyotrophic lateral sclerosis, motor neuron disease, prion disease, multiple system atrophy, ataxia, progressive multifocal leukoencephalopathy, corticobasal degeneration, Lewy body disease, multiple sclerosis, dementia, or Huntington's disease. The term “subject” may be used interchangeably with the terms “individual” and “patient” and includes human and non-human mammalian subjects.

The cell may be derived from a subject having a neurodegenerative disease. In exemplary embodiments, the cell is an induced pluripotent stem neuronal (iPSn) cell derived from a subject having a neurodegenerative diseases. The cell may be a primary cell or an immortalized cell, e.g. from a cell line.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. In some embodiments, the subject is responsive to therapy with a A to I RNA inhibitor, and include use in combination with one or more additional therapeutic agents. The term “treat” further includes the reduction in one or more symptom associated with a neurodegenerative disease, for example reduction or inhibition of neuron loss, reduction or inhibition of memory loss, reduction or inhibition of motor neuron loss or function, or reduction or inhibition of any symptom of neurodegenerative disease.

As used herein the term “effective amount” refers to the amount or dose of the small-molecule drug that provides the desired effect. In some embodiments, the effective amount is the amount or dose of the small-molecule drug, upon single or multiple dose administration to the subject, which provides the desired effect in the subject under diagnosis or treatment.

An effective amount can be readily determined by those of skill in the art, including an attending diagnostician, by the use of known techniques and by observing results obtained under analogous circumstances. In determining the effective amount or dose of small-molecule drug administered, a number of factors can be considered by the attending diagnostician, such as: the species of the subject; its size, age, and general health; the degree of involvement or the severity of the disease or disorder involved; the response of the individual subject; the particular small-molecule drug administered; the mode of administration; the bioavailability characteristics of the preparation administered; the dose regimen selected; the use of concomitant medication; and other relevant circumstances.

As used herein, the term “administering” an agent, such as a therapeutic entity like an inhibitor of A to I RNA editing, to a subject or cell, is intended to refer to dispensing, delivering or applying the substance to the intended target. In terms of the therapeutic agent composition, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. In some embodiments, the A to I RNA inhibitor may be administered more than once and or in combination with other methods of standard of care.

“Contacting a cell” refers to adding an agent, e.g. 8-aza to a culture medium or buffer containing the cell.

As used herein, a “small-molecule drug” is a molecule comprised of 20 to 100 atoms and has a molecular mass of less than 1000 g/mol or 1 kilodalton. Small-molecules drugs can typically be administered by a variety of routes (including orally) and can pass through cell membranes to reach intercellular targets.

The terms “antibody” refers to immunoglobulin molecules or other molecules which comprise an antigen binding domain. The term “antibody” is thus intended to include whole antibodies (e.g., IgG, IgA, IgE, IgM, or IgD), monoclonal antibodies, chimeric antibodies, and humanized antibodies. The term “antibody fragment” as used herein is intended to include any appropriate antibody fragment that displays antigen binding function, for example, Fab, Fab′, F(ab′)2, single chain variable fragment (scFv), Fv, dsFv, ds-scFv, Fd, mini bodies, single domain antibodies, antigen binding fragments, monobodies, and multimers thereof and bispecific antibody fragments.

Oligonucleotide inhibitors include, but are not limited to, antisense oligonucleotides (ASOs), small interfering RNAs (siRNA); short hairpin RNAs (shRNA); microRNAs (miRNA); artificial microRNAs (amiRNA); antisense oligonucleotides (ASO), including gapmers; and nucleic acid aptamers.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Example 1

In the following example, the inventors describe methods of dissolving pathological aggregates and reversing synaptic loss in subjects with neurodegenerative diseases.

We identify a novel pathology comprised of insoluble RNA binding proteins, NONO and SFPQ, in the nuclei of patient-derived iPSC midbrain neurons and synucleinopathy patient brains. NONO/SFPQ regulate gene expression as components of paraspeckles, punctate nuclear bodies of ˜0.3 mm in diameter that sequester mRNAs and proteins²⁷⁻³². NONO/SFPQ contain low complexity prion-like domains that allow for liquid-phase transitions into functional aggregates³³ that preferentially and directly bind to inosine-containing mRNAs²⁹. Adenosine-to-inosine (A-to-I) RNA editing occurs through an enzymatic reaction catalyzed by Adenosine Deaminase Acting on RNA (ADARs) 1 and 2, which bind and modify double stranded (ds) RNA sequences³⁴. Editing converts a stable A:U base pair into an unstable I:U base pair, causing dsRNA sequences to unwind into RNAs with single-stranded (ss)-like characteristics³⁵. As ADARs require dsRNA substrates³⁴, the presence of inosine directly indicates a double-stranded structure in vivo. Further, A-to-I editing of dsRNA predominantly occurs within primate-specific inverted Alu repetitive elements of introns and 3′ untranslated regions (UTRs)³⁶. Although the biological function is not completely understood, previous work has associated A-to-I editing with reduced gene expression and nuclear retention^{29,37-39}. Under physiological conditions, a few select mRNAs have been shown to escape nuclear retention through binding competition between NONO and STAUFEN1, or CARM1 modification of NONO^{40,41}. In addition to binding A-to-I edited transcripts, paraspeckles also control A-to-I editing through sequestration of soluble SFPQ, a required transcriptional enhancer of Adenosine Deaminase Acting on RNA-3 (ADAR3; ADARB2)^{42,43}. ADAR3 is brain specific and despite its name, has no detectable catalytic activity⁴⁴. Instead, ADAR3 binds to dsRNA sequences in competition with the enzymatically active ADARs 1 and 2, and inhibits A-to-I editing⁴⁴⁻⁴⁷. By inhibiting A-to-I editing, overexpression of ADAR3 promotes dsRNA structure, while its depletion can reduce dsRNA structure, consistent with the effect of inosine on destabilizing dsRNA⁴⁷.

The importance of RNA editing in the brain is well-established, since it is essential for human brain development, and axon/synaptic transcripts are heavily targeted by ADARs⁴⁸⁻⁵⁴. Recent studies have also shown that A-to-I RNA editing is altered in blood samples and brain tissue of PD patients, providing in vivo evidence that RNA editing changes are associated with disease^{55,56}. Furthermore, NONO and SFPQ have also been linked to neurodegenerative disease. Genetic variants of SFPQ are associated with motor neuron disease and synaptic abnormalities^{57,58}, and SFPQ aggregates occur in amyotrophic lateral sclerosis⁵⁸. Loss of NONO in humans causes a severe neurodevelopmental disorder characterized by intellectual disability and loss of synapses, establishing its significance in maintaining neuronal health⁵⁹. Here, we identify and characterize the downstream functional consequences of nuclear inclusions comprised of NONO/SFPQ and edited mRNA.

### Results

Proteomic Analysis of Aggregated Proteins in PD Patient iPSn

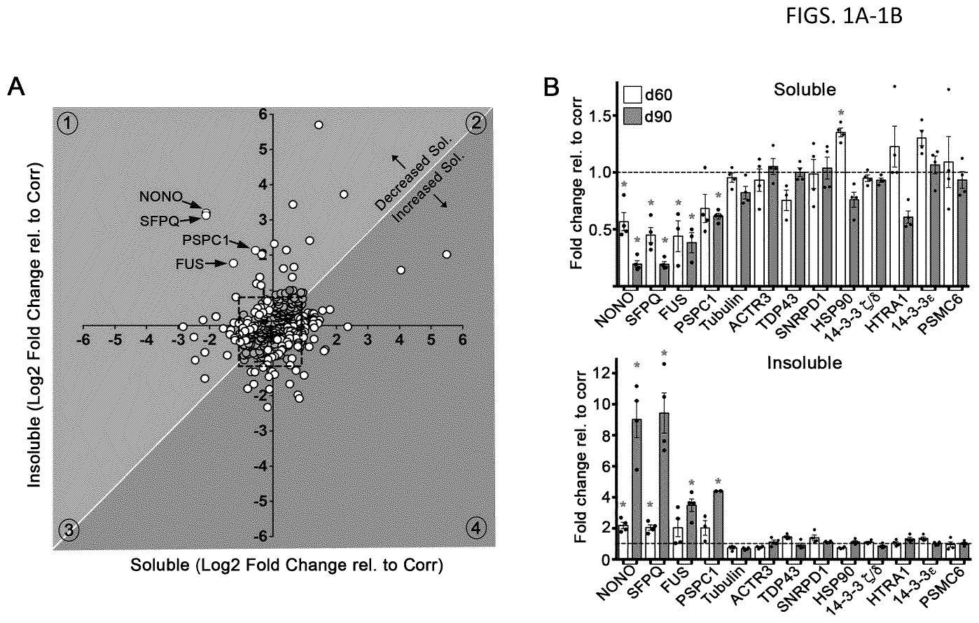

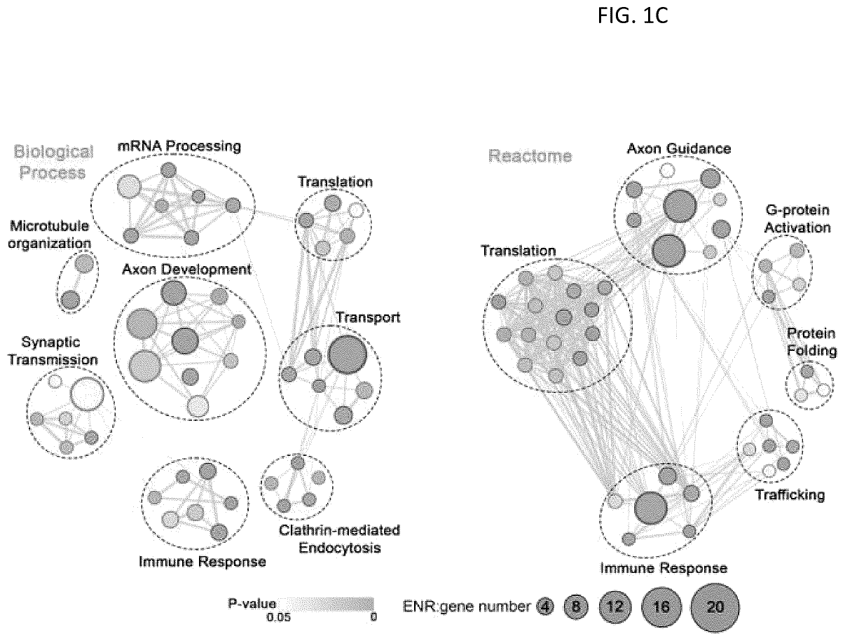

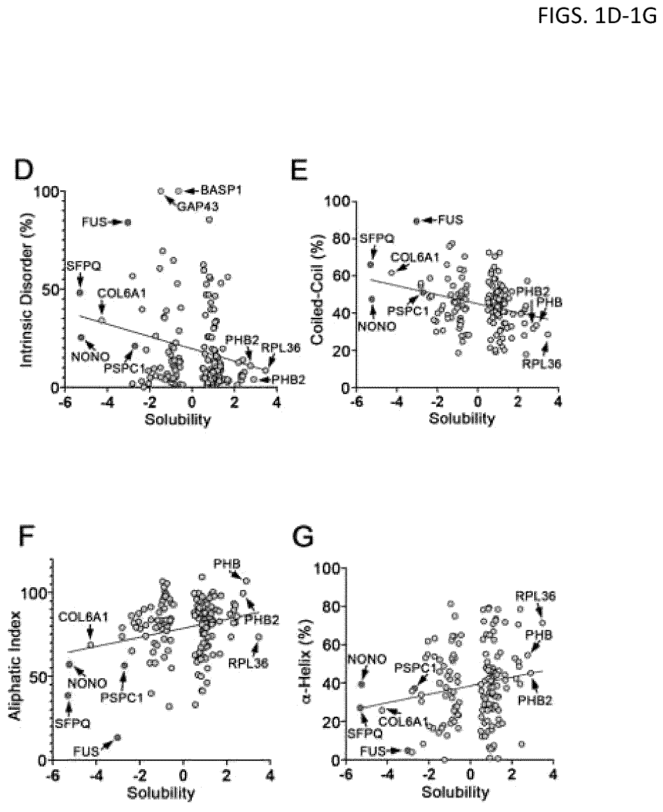

To gain a comprehensive understanding of protein solubility changes in synucleinopathies, we performed sequential extractions followed by quantitative mass spectrometry⁶⁰ on established iPSC-midbrain culture lines (iPSn) derived from PD patients harboring SNCA mutation A53T and matching isogenic controls^{7,19,24,61-63} PD iPSn are mature at day (d) 60 post-differentiation, and gradually accumulate insoluble α-syn between d60-90, followed by neurite degeneration after d100^{19,64}. Proteome aggregation was examined by searching for proteins that shifted from their normal soluble state into the insoluble fraction (FIG. 1A, quadrant 1). Most detected proteins showed no change in solubility (gray plots, FIG. 1A) however a small subset of proteins underwent a significant shift into the insoluble state (white plots in quadrant 1, FIG. 1A). The largest shift occurred in two nuclear proteins involved in gene regulation and paraspeckle formation, NONO and SFPQ (FIG. 1A). Validation studies across multiple culture batches showed that NONO/SFPQ insolubility progressively increased with culture age between d60 and d90, revealing up to a 9-fold elevation of insoluble protein (FIG. 1B).

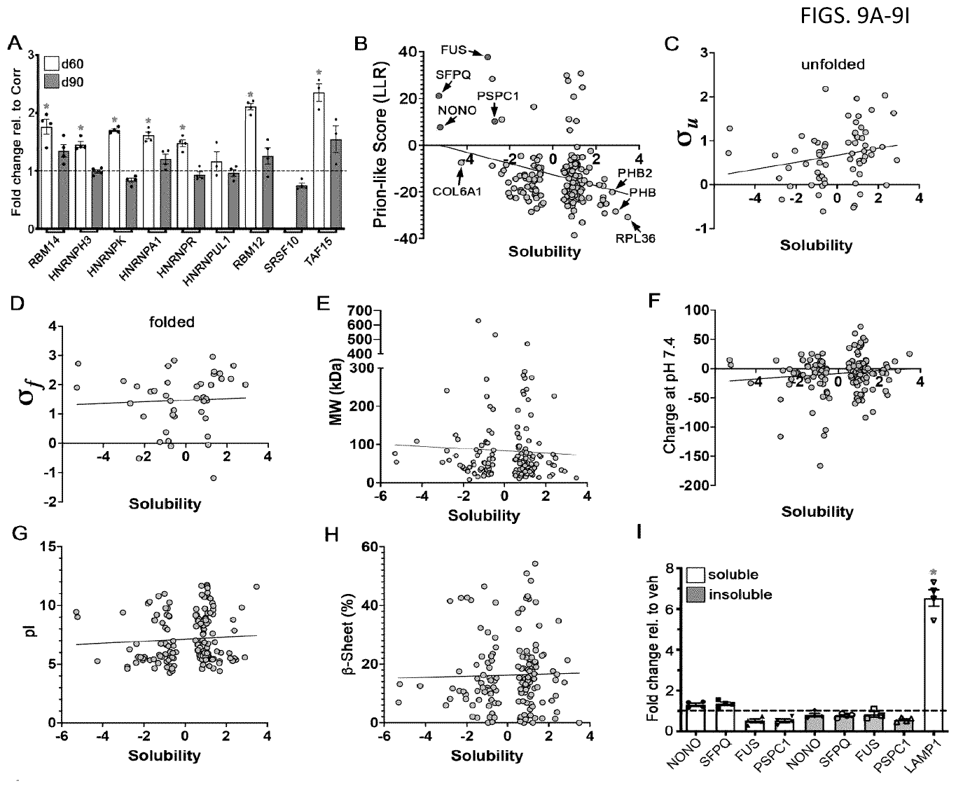

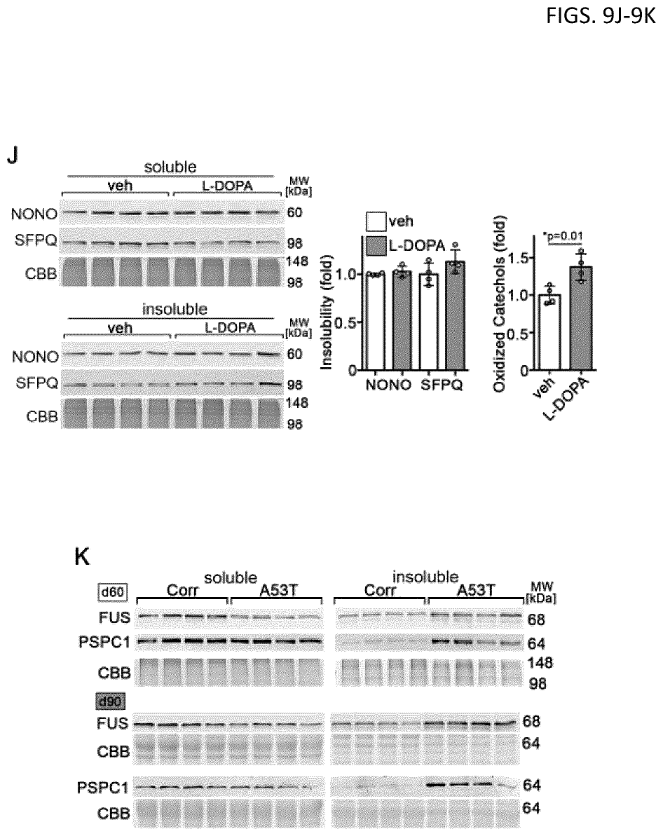

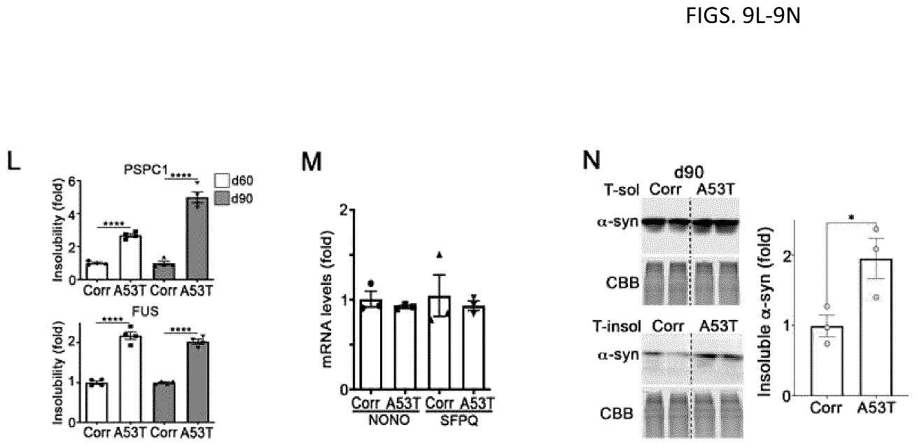

Gene ontology analysis of insoluble proteins showed that the most prominent solubility shifts occurred in categories of mRNA processing, translation, and axon development (FIG. 1C). Other RNA binding proteins, including FUS and PSPC1, were also elevated in PD iPSn although not as dramatically as NONO/SFPQ (FIG. 1A, B). Protein solubility changes showed specificity, since highly stable structural proteins including actin, tubulin, and the ALS-linked RNA-binding protein TDP-43 were not dramatically changed (FIG. 1B). We quantified other paraspeckle proteins that are essential for their formation, including RNA binding proteins RBM14, HNRNPH3, HNRNPK²⁷, as well as HNRNPA1 that has been linked to other degenerative conditions⁶⁵. Some, but not all, of these proteins were increased in the insoluble fraction at d60 by ˜2 fold, however none of these proteins progressively accumulated with culture age as was observed with NONO/SFPQ (FIG. 9A).

To understand the basis for solubility changes in PD iPSn, we next examined physicochemical properties of proteins with significantly altered solubility (all white plots in FIG. 1A). We found significant negative correlations between protein solubility and intrinsic disorder⁶⁶ metastable coiled-coil domains that provide structural flexibility⁶⁷, and low complexity prion-like domains⁶⁸ (FIG. 1D, E; FIG. 9B). SFPQ was previously noted to contain highly extended coiled-coil domains that are essential for its self-assembly⁴³. Significant positive correlations were noted with aliphatic index (a measure of thermostability⁶⁹) and alpha-helical content (FIG. 1F, G). Comprehensive analysis examined correlations with supersaturation values (a metric that incorporates both expression levels and intrinsic aggregation propensity²⁶), protein size, charge, isoelectric point, and beta-sheet propensity, but no significant relationships were found (FIG. 9 C-H). These data indicate that protein solubility changes in PD iPSn can be partly explained by basic physicochemical properties, where proteins with flexible, metastable domains appear to be more sensitive to aggregation.

We next determined if NONO/SFPQ aggregation occurs upon general cellular stress associated with PD, including lysosomal dysfunction^{22,24} and oxidant stress^{70,71}. Healthy control iPSn at d60 were treated with lysosomal inhibitors for 1 month, followed by quantitative MS analysis. This showed a dramatic accumulation of LAMP1 protein consistent with lysosomal dysfunction, however no changes in the levels or solubility of NONO, SFPQ, FUS, or PSPC1 were observed (FIG. 9I). We next stressed iPSn with excess levels of the catecholamine L-DOPA, which induces oxidation in midbrain DAergic neurons^{72,73}. L-DOPA treatment significantly enhanced oxidation as expected⁷², but had no effect on NONO or SFPQ solubility (FIG. 9J). These data suggest that solubility changes are specific to synucleinopathy patient neurons rather than generalized cellular stress.

Nuclear Inclusions of NONO/SFPQ in Patient iPSn and Synucleinopathy Patient Brain.

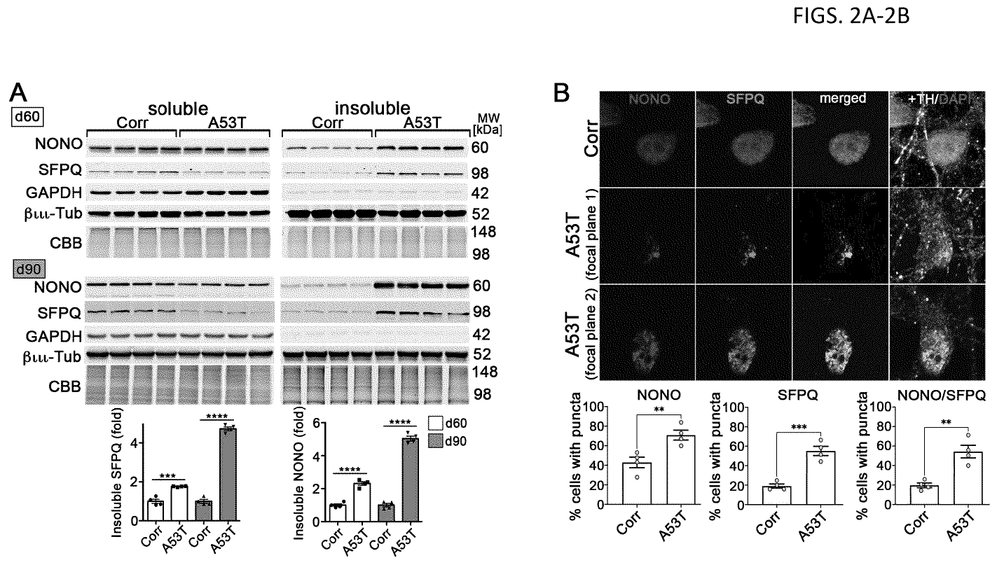

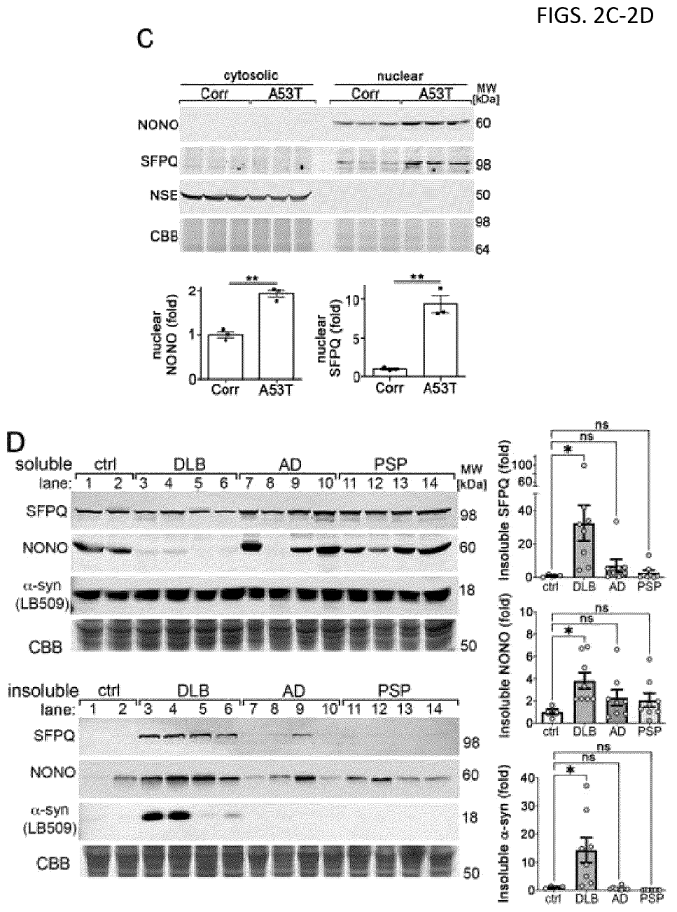

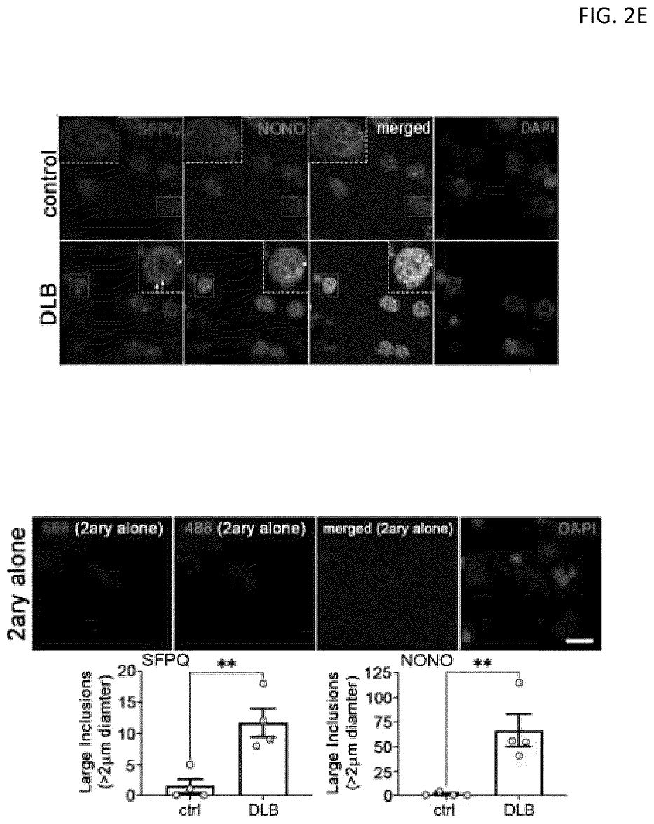

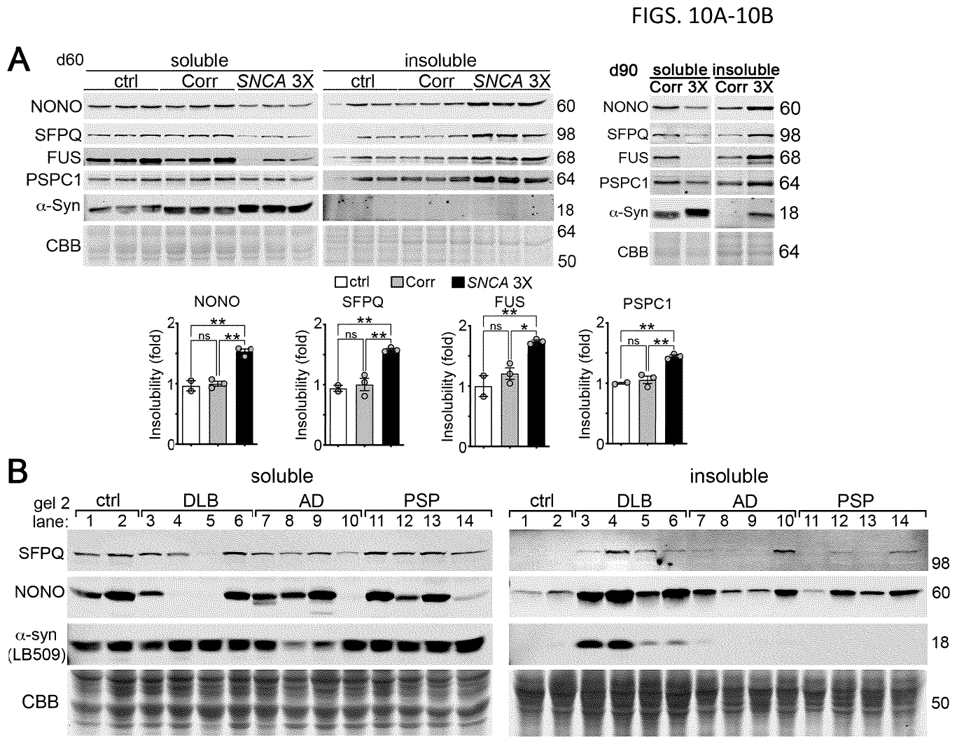

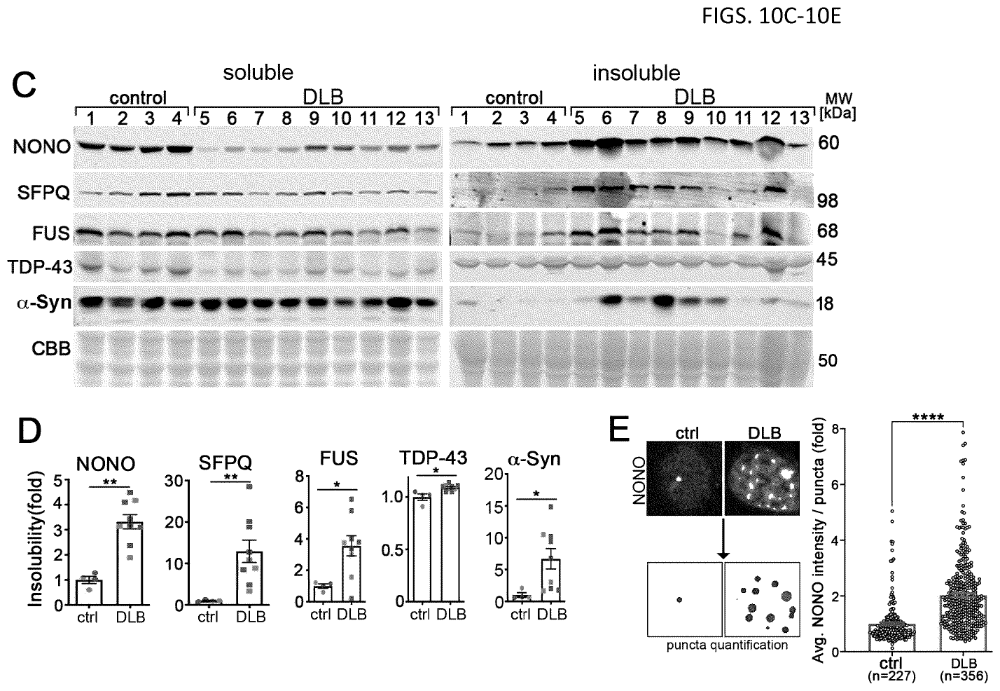

To validate the proteomic results, we analyzed iPSn lysates by western blot and fixed cultures by immunofluorescence staining. Western blot showed an elevation of NONO, SFPQ, FUS, and PSPC1 in the insoluble fraction of d60 and d90 cultures that exacerbated over time, and a reduction in the soluble fraction (FIG. 2A, FIG. 9K, L). Analysis of mRNA showed no changes in the levels of NONO or SFPQ mRNA, indicating that the accumulation occurred post-transcriptionally (FIG. 9M). In comparison, the levels of insoluble α-synin PD iPSn were about 2 fold higher in PD iPSn (FIG. 9N). Next, the location of NONO/SFPQ aggregates was examined by immunofluorescence staining of fixed PD cultures and nuclear/cytoplasmic fractionation. NONO/SFPQ were found in an evenly distributed, diffuse pattern in the nuclei of isogenic control neurons (FIG. 2B). In PD patient iPSn, confocal analysis showed increased NONO/SFPQ puncta in the nucleus that occurred as either large inclusions (FIG. 2B, A53T, focal plane-1), or small puncta (FIG. 2B, A53T, focal plane-2). Importantly, no changes in tyrosine hydroxylase staining (white) occurred between controls and PD iPSn (FIG. 2B). Biochemical fractionation/western blot confirmed nuclear accumulation of NONO and SFPQ (FIG. 2C). Finally, analysis of a distinct synucleinopathy patient line that harbors a triplication of the wild-type (wt) SNCA locus (3X)¹⁹ revealed elevated insoluble NONO, SFPQ, FUS, and PSPC1 at day 60, even in the absence of detectable insoluble α-syn at this time point (FIG. 10A). Insolubility of these proteins persisted to day 90 in this line (FIG. 10A, right).

To determine the relevance of these findings to human disease, we analyzed synucleinopathy patient brain. Biochemical analysis of cortical samples from pathologically confirmed regions of sporadic DLB and age/gender matched controls showed that insoluble NONO/SFPQ was dramatically elevated, with an average increase of 30-fold, and the most severe patient exhibiting a 100-fold increase in SFPQ insolubility (FIG. 2D; FIG. 10B). Analysis of two distinct neurodegenerative diseases involving either intermediate levels of α-synpathology (AD with minor α-synpathology) or the tauopathy, progressive supranuclear palsy (PSP), showed no statistical difference in insoluble NONO/SFPQ, although the average of NONO trended slightly higher in AD and PSP samples (FIG. 2D; FIG. 10B). This suggests that NONO/SFPQ aggregation does not occur from general neurodegeneration. We then validated these data using a separate cohort of post-mortem tissue obtained from a distinct brain bank. Elevated levels of insoluble NONO and/or SFPQ was detected in all of the DLB samples analyzed and showed an average increase of ˜12-fold in SFPQ and ˜4-fold increase in NONO (FIG. 10C, D). In comparison, ˜70% of DLB samples showed an increase in insoluble α-syn (FIG. 2D, FIG. 10C, D), using the highly reliable and sensitive antibody LB509 that was generated directly against purified Lewy bodies 74 Analysis of insoluble FUS showed a 4-fold elevation in DLB brain, while only a subtle change was found with TDP-43 (˜10% increase) (FIG. 10C, D).

Confocal imaging of fixed DLB brain revealed nuclear inclusions of NONO/SFPQ that occurred in a punctated pattern (FIG. 2E). Partial colocalization of NONO and SFPQ puncta was observed (FIG. 2E, arrows), although large inclusions >2 μm in diameter comprised of individual NONO or SFPQ proteins also commonly occurred (FIG. 2E, arrowhead). The large size of these inclusions compared to physiological paraspeckles (˜0.3 mm), in addition to separate inclusions of NONO or SFPQ, suggests that the inclusions are not paraspeckles. Quantification of inclusion diameter showed that the nuclei of DLB brains contained a higher number of large inclusions (>2 um diameter), and also contained a higher number of total puncta per cell that exhibited a stronger intensity (a reflection of both stronger pixel intensity and larger puncta size) (FIG. 2E, S2E). Collectively, biochemical and pathological data validate that accumulation of insoluble NONO/SFPQ occurs in synucleinopathy patient brain, supporting pathological relevance for the pathway in disease.

### Interaction and Co-Aggregation of α-Syn and SFPQ In Vitro and In Vivo.

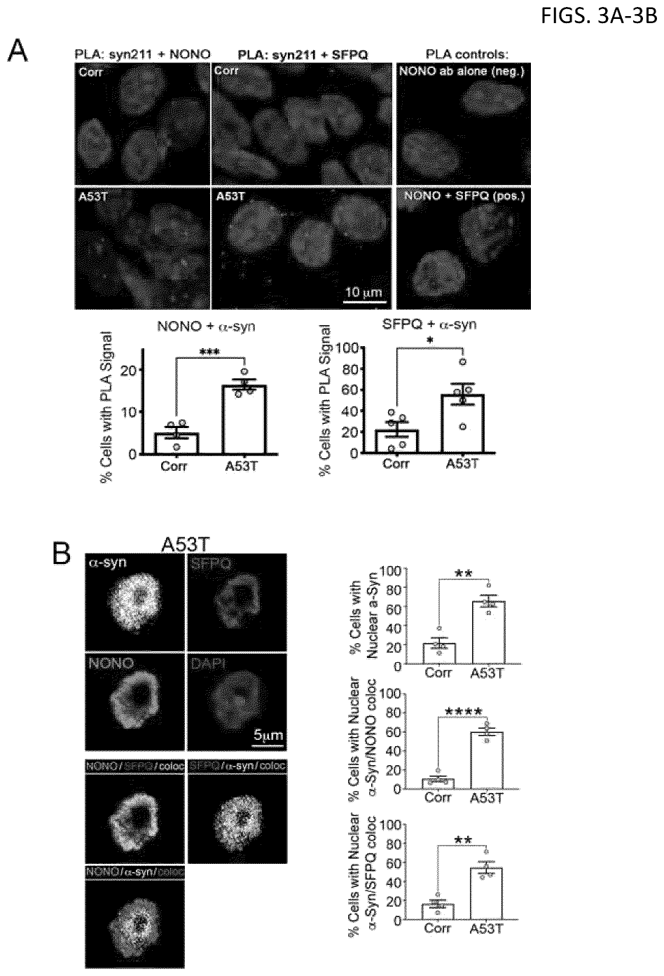

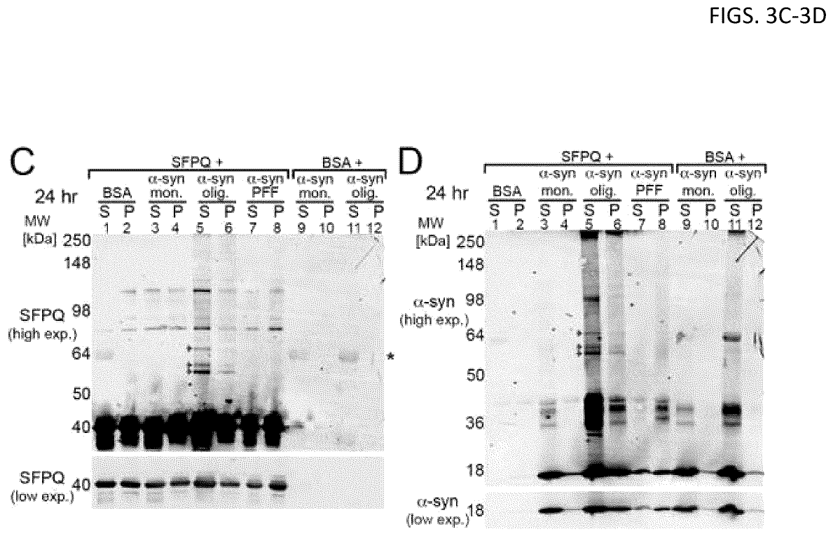

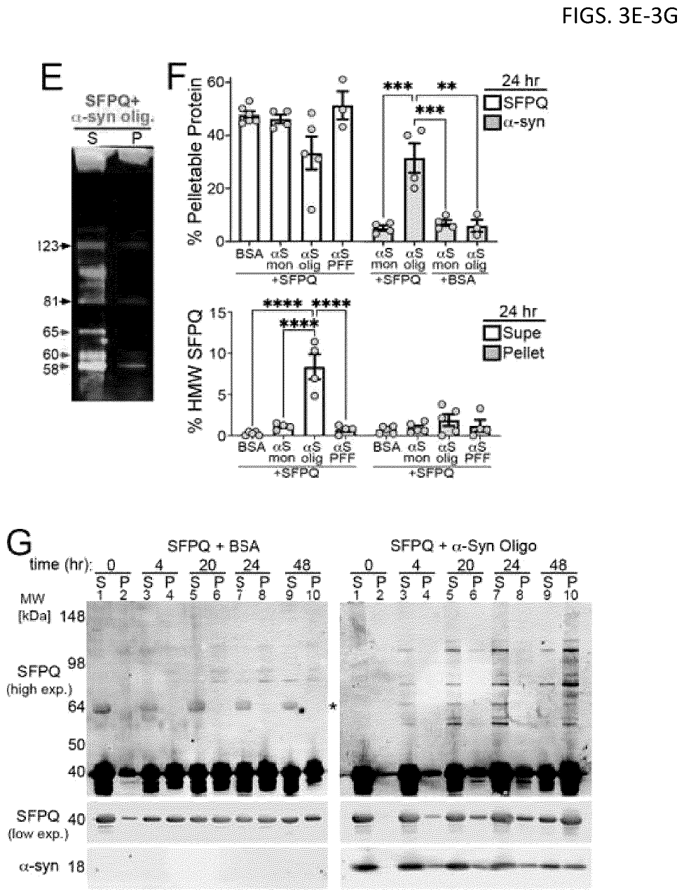

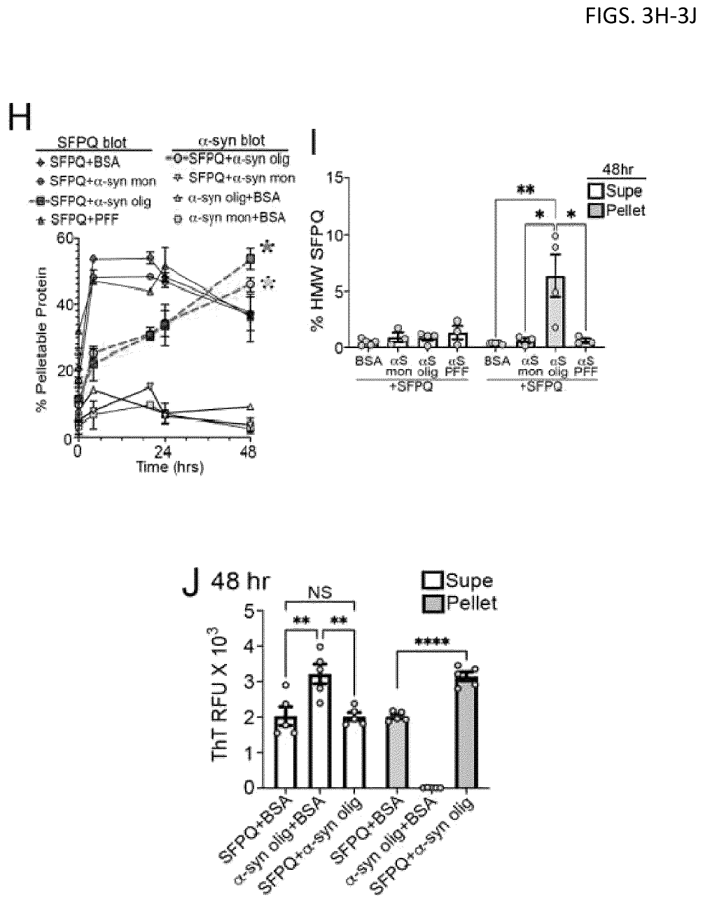

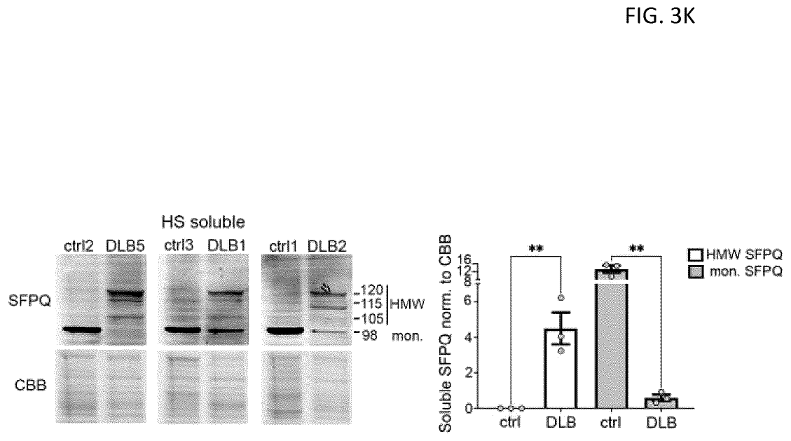

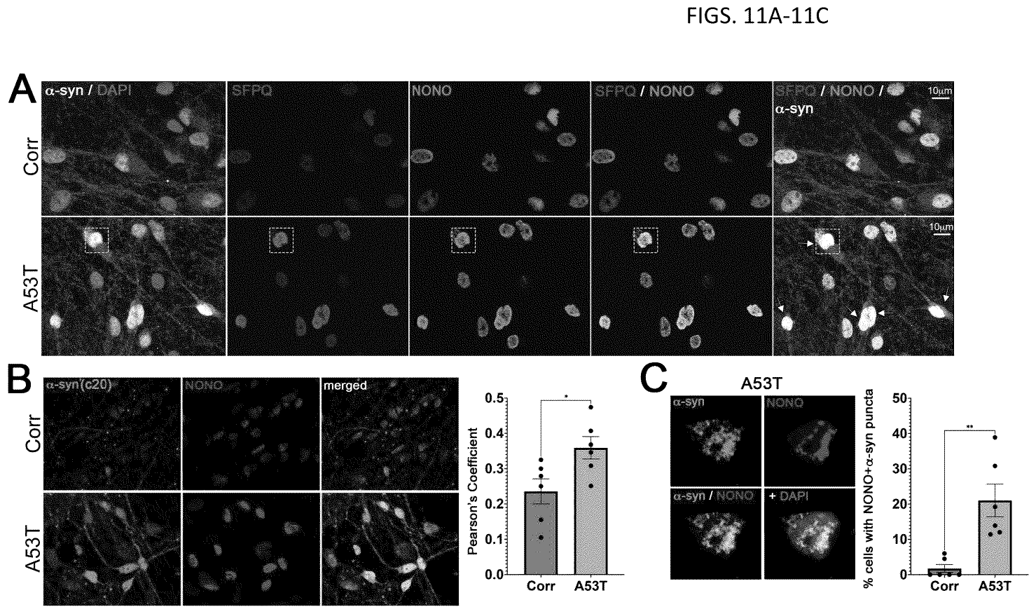

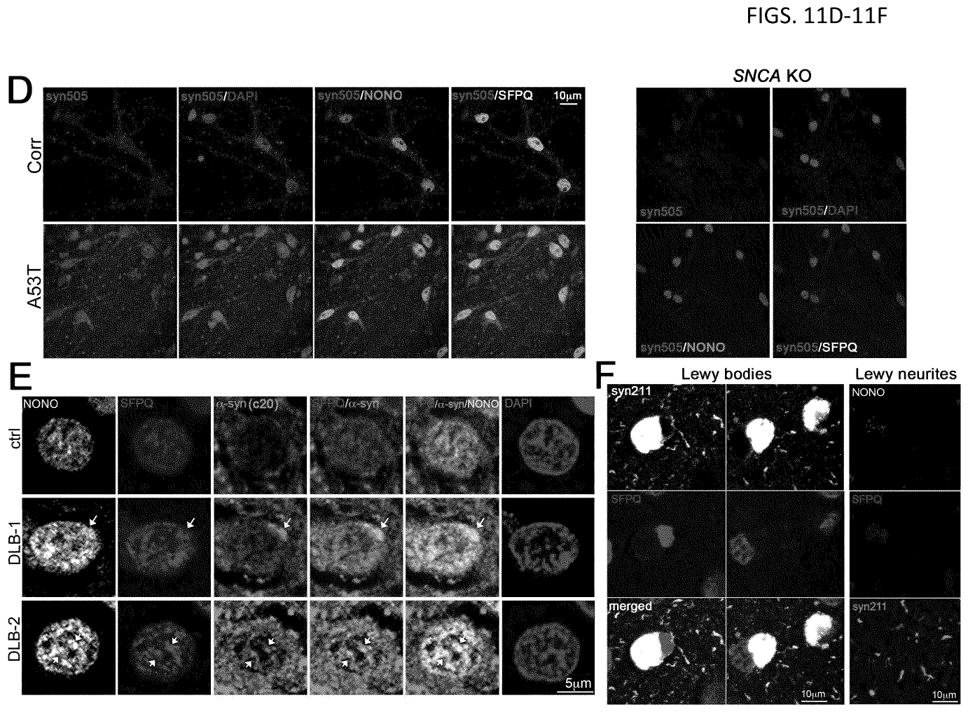

To gain insight into the mechanism of NONO/SFPQ aggregation, we tested if α-syn associates with NONO or SFPQ in the nucleus. This hypothesis is based on data showing α-syn is present in the nucleus in model systems⁷⁵, in synucleinopathy patient brain^{76,77}, and previous interactome studies show that α-syn interacts with NONO and SFPQ⁷⁸. We confirmed the interaction of α-syn with NONO/SFPQ by proximity ligation assays (PLA) (FIG. 3A), and confocal microscopy showed that ˜50% of cells contained nuclear α-syn colocalized with either NONO or SFPQ (FIG. 3B, FIG. 11A). Nuclear accumulation was confirmed with three independent α-syn antibodies that react with both N and C-terminus of the protein including syn211 (residues 120-125), C20 (polyclonal c-term), and syn505 (residues 2-4) (FIG. 3B, FIG. 11B-D). Syn505 preferentially detects pathological, soluble cross-linked oligomers⁷⁹ and was generated against oxidized/nitrated α-syn epitopes 9. α-syn and NONO/SFPQ colocalization was observed as a discrete diffuse pattern, as shown by 1 um confocal slices taken throughout the nucleus of PD iPSn (FIG. 3B, FIG. 11A). In focal planes beyond the nucleus, α-syn was also detected in a punctated pattern within neuronal extensions of both controls and PD iPSn, consistent with its expected synaptic localization (FIG. 11B, D). However colocalization with NONO/SFPQ was mainly restricted to the nucleus. We also observed colocalization within discrete puncta in ˜20% of PD iPSn but not controls (FIG. 11C), although most cells with α-syn-SFPQ/NONO colocalization (˜80%) exhibited a diffuse staining pattern.

Analysis of post-mortem DLB brain showed that α-syn colocalized with NONO/SFPQ in the nucleus, while controls showed little colocalization (FIG. 11E). Importantly, NONO/SFPQ inclusions were not detected in Lewy bodies or neurites that histopathologically define synucleinopathies (FIG. 11F). These data indicate that non-Lewy body α-syn-species accumulate within the nucleus of DLB brain and colocalize with NONO/SFPQ, suggesting that nuclear forms of α-syn may interact and trigger their aggregation in disease.

Since sequential extractions and PLA data indicated that SFPQ was more dramatically affected by α-syn compared to NONO in DLB brain (FIG. 2D; FIG. 3A), we next focused on testing if α-syn and SFPQ could directly interact and influence each other's aggregation using purified recombinant proteins. A 40 kDa truncated form of SFPQ was utilized that includes the metastable coiled-coil motif essential for polymerization, given that it was not possible to purify the full length SFPQ at quantities required for in vitro aggregation studies 43. α-syn and SFPQ were incubated at 1 mg/ml under physiological conditions (pH 7.4, 37° C.) with sample agitation, and aggregation was assessed by centrifugal sedimentation/western blot analysis. In control conditions using bovine serum albumin (BSA) to control for molecular crowding, ˜50% of SFPQ converted from the soluble supernatant (S) into pellet (P) fraction at 24 hours (hrs) (FIG. 3C, lanes 1, 2; FIG. 3F). Analysis of α-syn monomers+BSA showed that ˜95% of α-syn remained in the supernatant (FIG. 3D, lanes 9, 10; FIG. 3F), consistent with previous studies of α-syn aggregation at 1 mg/ml⁸⁰. Mixing different α-syn species with SFPQ, including α-syn monomers, oligomers (generated by lyophilization⁸¹), or fragmented pre-formed fibrils (PFF) showed that only α-syn oligomers influenced SFPQ aggregation by inducing high molecular weight (HMW) SFPQ (ca. ˜58 and 123 kDa) in the supernatant fraction (FIG. 3C, lane 5; FIG. 3F). Colocalization analysis on western blots indicated that species at 58, 60, and 65 kDa contained co-migrating SFPQ and α-syn species that were not dissociated by SDS and heat (FIG. 3E, F). Even though previous work showed that α-syn oligomers generated by lyophilization are kinetically trapped in the soluble state and resist conversion into pelletable aggregates⁸¹⁻⁸⁴, we found that SFPQ could trigger the conversion of α-syn oligomers into pelletable aggregates, thus demonstrating a reciprocal relationship between SFPQ and α-syn (FIG. 3D, lane 6). Note that the HMW smear of α-syn in the soluble fraction confirms that oligomers were generated successfully by the lyophilization protocol (FIG. 3D, lane 5). Kinetic analysis of SFPQ aggregation between 0 and 48 hrs showed that oligomeric α-syn kinetically stabilized soluble HMW SFPQ species up to 24 hr (FIG. 3G, lanes 7, 8; FIG. 3H). Between 24-48 hr, soluble HMW SFPQ oligomers were then converted to pelletable aggregates, indicating they are aggregation competent (FIG. 3G, lane 9, 10; FIG. 3H, I). Consistent with a reciprocal effect on aggregation of the two proteins, the sedimentation of α-syn oligomers and SFPQ became synchronized when mixed (red dotted lines), an effect that was not observed when SFPQ was mixed with either α-syn monomers, PFFs, or BSA (FIG. 3H). We next tested if mixing α-syn oligomers and SFPQ induced conformational changes using the amyloid binding dye thioflavin T (ThT). In supernatant fractions at 48 hrs, α-syn oligomers reacted slightly more with ThT compared to SFPQ and α-syn/SFPQ mixtures (FIG. 3J, open columns). In contrast, pelletable α-syn/SFPQ mixtures showed higher ThT fluorescence compared to either protein alone, indicating increased amyloid content (FIG. 3J, gray columns). These data indicate that α-syn oligomers affect SFPQ aggregation by initial kinetic stabilization of soluble HMW SFPQ species followed by their aggregation into pelleted ThT-positive aggregates at 48 hr.

To determine if HMW forms of SFPQ occur in DLB brain, soluble brain extracts were analyzed by Western blot analysis. While only a single 98 kDa SFPQ species was detected in control brains representing the full-length protein, DLB brains showed HMW SFPQ species migrating at 120, 115, and 105 kDa (FIG. 3K). These data indicate that HMW forms of SFPQ exist in vivo and are associated with disease, suggesting that SFPQ aggregation in DLB brain may proceed through initial formation of soluble, HMW SFPQ followed by aggregation into large insoluble inclusions.

Depletion of ADAR3 and Increased A-to-I Editing of RNA in PD iPSn and DLB Brain

We next determined the pathophysiological consequences of nuclear NONO/SFPQ inclusions. Soluble SFPQ regulates RNA editing as a required transcriptional activator of ADAR3^{42,43}, a brain specific inhibitor of RNA A-to-I editing^{44-47,85}. NONO/SFPQ complexes also bind and sequester A-to-I edited mRNAs within paraspeckles^{29,37-39,86}. Given that multiple connections exist between NONO/SFPQ and A-to-I editing, and the effects of editing in synucleinopathies are currently unknown, we examined this pathway in more detail. We hypothesized that the effects of NONO/SFPQ inclusions may be twofold: 1) SFPQ aggregation may cause a loss of its function by sequestering soluble, functional SFPQ, resulting in reduced ADAR3 expression and increased A-to-I editing; and 2) NONO/SFPQ inclusions may aberrantly bind and sequester A-to-I edited transcripts that are essential for neuronal health, resulting in reduced gene expression and neurodegeneration.

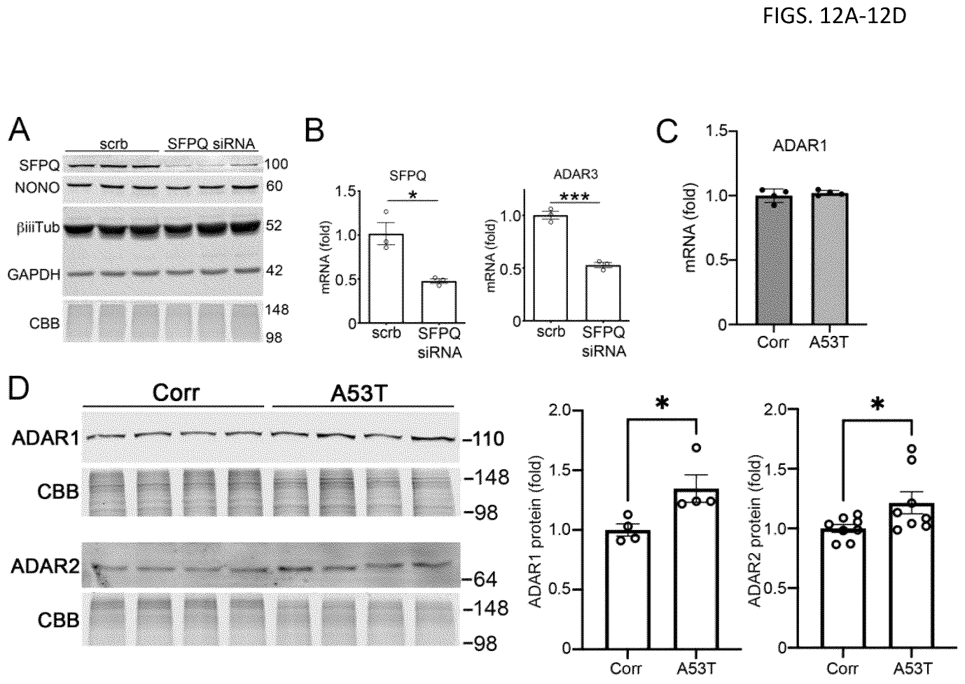

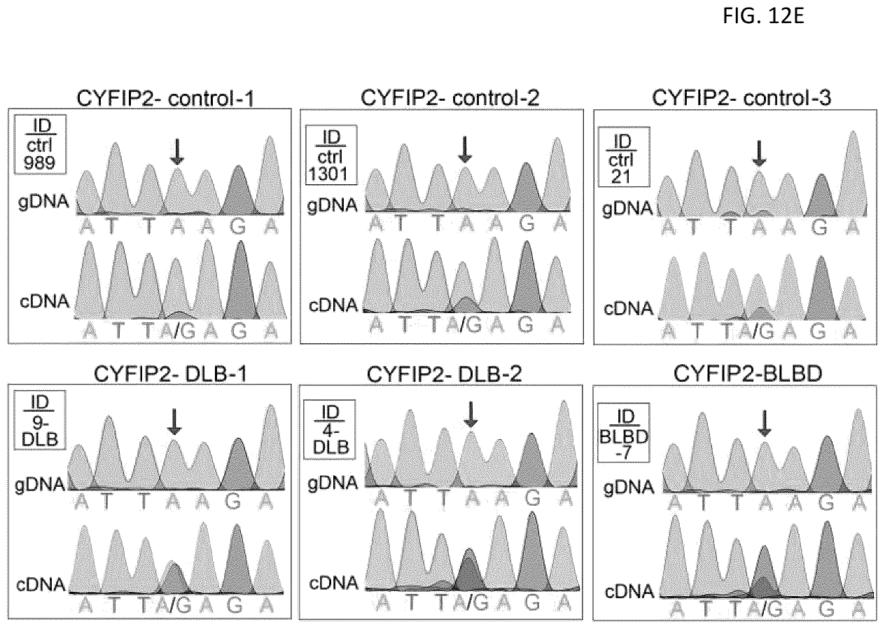

To address the first scenario, we determined the expression levels of ADAR3. We first confirmed that soluble SFPQ is essential for ADAR3 expression in healthy control iPSC-neurons by siRNA knock-down of SFPQ. This resulted in dramatic reduction of ADAR3 mRNA (FIG. 12A, B), which is consistent with previous findings in other non-neuronal cell types^{42,43}. Next, using PD iPSn, we directly tested the promoter activity of ADAR3 with a luciferase reporter and found that it was dramatically reduced (FIG. 4A). Endogenous ADAR3 mRNA and protein were also reduced by ˜75% (FIG. 4B, C). This effect was specific to ADAR3, since the mRNA level of ADAR1 did not change, and the protein levels of ADARs 1 and 2 were mildly increased in PD iPSn (FIG. 12D). We assessed the in vivo relevance of these findings by analyzing DLB brain lysates by western blot, which revealed a ˜70% reduction in ADAR3 compared to healthy controls (FIG. 4D). Given that soluble SFPQ is essential for ADAR3 expression (FIG. 12A)^{42,43}, these data suggest that insoluble aggregates of SFPQ confer loss-of-function in PD iPSn and DLB brain.

Since ADAR3 is an established regulator of A-to-I editing⁴⁴⁻⁴⁶, we next determined if RNA editing levels were altered in PD patient iPSn. Editing events are detected by comparing genomic DNA with cDNA sequences and appear as adenosine-to-guanosine (A-to-G) mismatches, since inosine is interpreted as guanosine by the sequencing machinery. We started by assessing edit levels in two select transcripts, Cytoplasmic FMR1-interacting protein 2 (CYFIP2) and Calcium-dependent secretion activator 1 (CADPS; CAPS-1), since previous work showed they have a high confidence of A-to-I editing occurrences in vivo⁵³ (e.g. no evidence for A-G variants at the genomic DNA, A-to-I editing levels of ≥50% in cDNA from multiple brain samples, and evidence that editing in this region is conserved in evolution). Furthermore, CYFIP2 and CADPS are neuronally expressed, essential for synaptic/axonal health⁸⁷⁻⁸⁹, and our proteomic analysis showed that both proteins are reduced in PD iPSn while total mRNA levels were unchanged (FIG. 4E). Sequencing revealed that the percentage of A-to-I edited CYFIP2 and CADPS transcripts were elevated in PD iPSn (FIG. 4F, G). Furthermore, restoring the expression of ADAR3 by lenti-viral infection of PD iPSn reduced CYFIP2 editing (FIG. 4H). This indicates that A-to-I editing is increased in PD patient iPSn through ADAR3 depletion.

We next measured A-to-I editing in human DLB brain. The percentage of edited CYFIP2 transcripts significantly declined with age in the cortex of healthy individuals, which is a normal physiological response and is consistent with previous findings⁹⁰ (FIG. 4I, left). However in DLB cortex, A-to-I editing remained elevated as a function of age, and the trend significantly differed from controls (FIG. 4I, right). Quantification of the combined age groups indicated a significant increase in A-to-I editing in DLB (FIG. 4J), while closer examination of the population of age 65 and older showed a more dramatic elevation in sporadic DLB and 2-fold elevation in familial PD patients that harbor SNCA mutations (FIG. 4K). In many DLB cases, the edited transcript represented ≥50% of the total proportion of expressed transcripts (FIG. 12E). Interestingly, analysis of cortical samples from early-stage sporadic patients with only brainstem Lewy bodies (BLBD) but free of cortical Lewy bodies, showed a 2-fold increase in the percentage of A-to-I edited mRNA (FIG. 4K). This suggests that increased A-to-I editing may occur during the earliest stages of pathology prior to the formation of insoluble, microscopically visible Lewy inclusions. This is consistent with immunostaining data showing colocalization of diffuse, non-Lewy body α-syn with NONO/SFPQ (FIG. 3B, FIG. 11), and soluble α-syn oligomers that trigger SFPQ conversion into aggregates (FIG. 2). This is further supported by the fact that increased editing was observed prior to neurodegeneration in d60 PD iPSn (FIG. 4F, G), suggesting that it may contribute to early, initial stages of synaptic degeneration that characterize early-stage PD pathology^{9,12,91}.

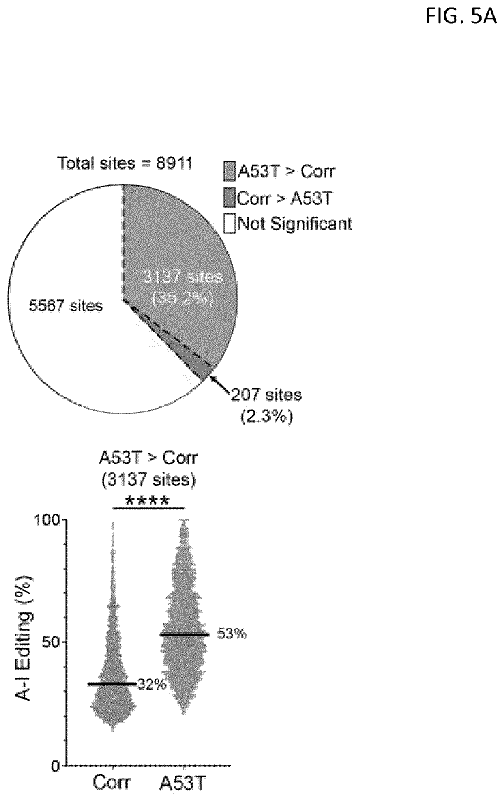

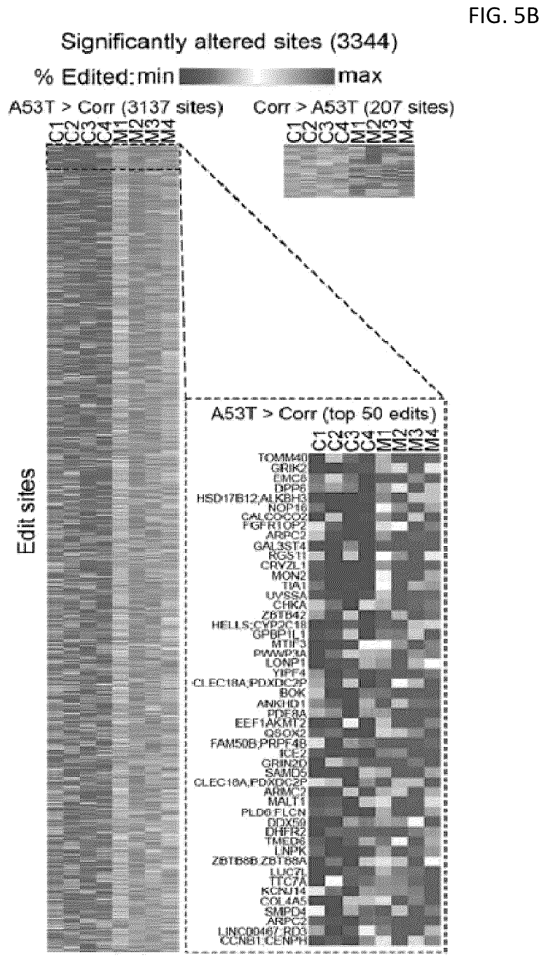

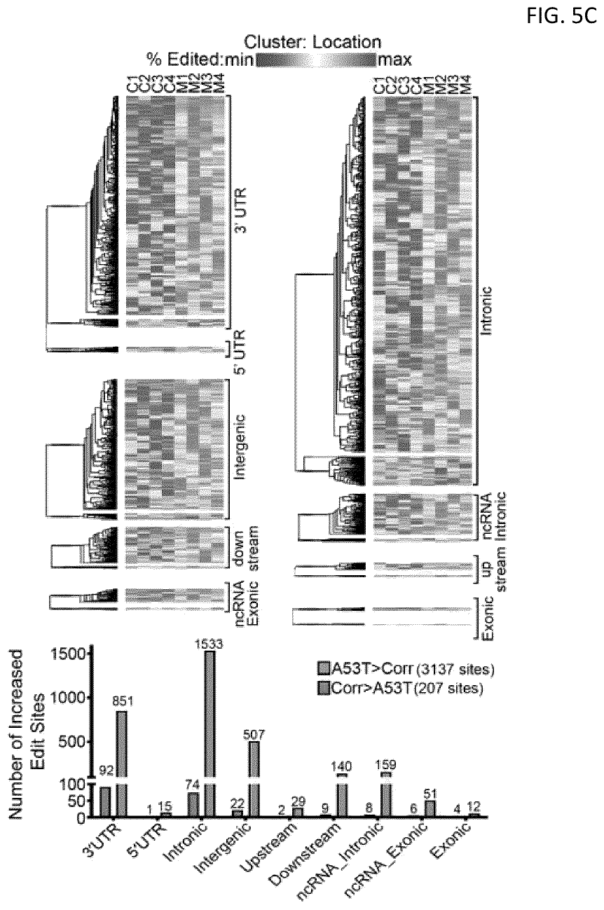

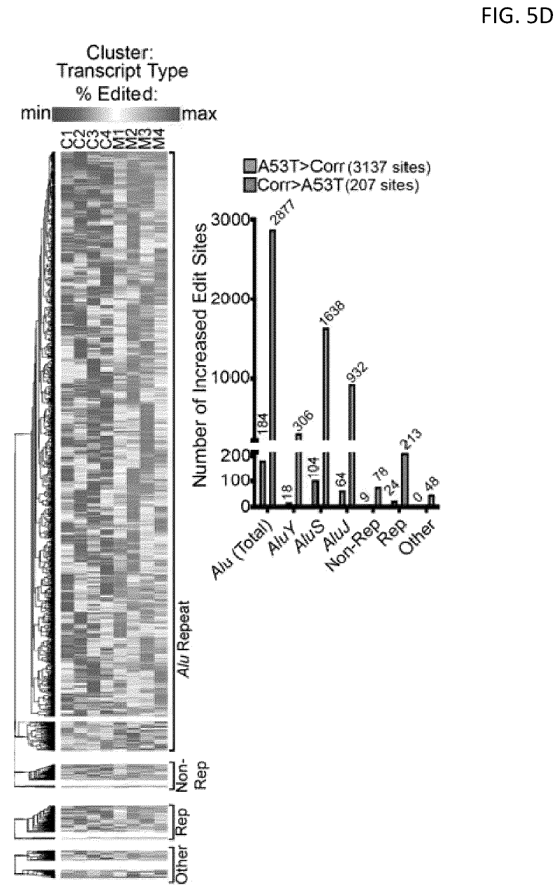

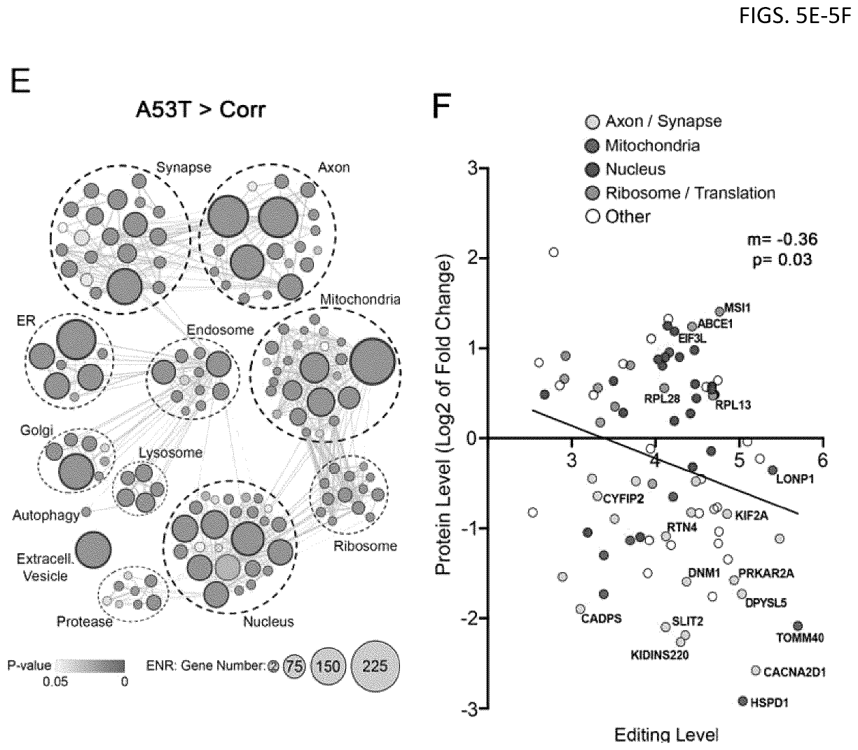

We expanded our analysis of A-to-I editing events in PD iPSn and determined if global changes occur by RNA-seq. Poly-A mRNA was isolated from PD and isogenic corrected iPSn, then compared to whole genome sequencing data. Using a 300M base-pair read depth for RNA-seq, we identified 8,911 A-to-I edit sites, calculated as percent of total transcripts. Of these, 7090 sites (80%) were elevated in PD iPSn, with 3,137 sites that reached statistical significance (FIG. 5A). Within all sites that were significantly changed, an average increase of 21% in the conversion of A-to-I was observed in PD iPSn compared to isogenic controls (FIG. 5A, bottom). In contrast, only a small portion of edit sites (207 sites, or 2.3%) were elevated in isogenic controls (FIG. 5A, B). A heat map of the editing changes in 4 individual culture well replicates revealed robust and consistent elevations in all sites identified (FIG. 5B). Examination of the top 50 increased edited transcripts in PD iPSn revealed changes in regulators of synapse function and ion channels (GRIK2, GRIN2D, DPP6, KCNJ14), mitochondrial proteins (TOMM40, LONP1, MTIF3), endoplasmic reticulum (EMC8, LNPK, TMED6), and RNA processing proteins (TIA1, ICE2, LUC7L, UVSSA). By focusing only on significantly changed edited sites, dendrograms showed that editing was most frequently elevated within intronic (1533 sites), 3′UTR (851 sites), and intergenic regions (507 sites) in PD iPSn (FIG. 5C). By contrast, only 12 edit sites were found to be elevated in exonic regions (FIG. 5C). The majority of increased editing (92% or 2877 sites) occurred in human specific, inverted Alu repetitive elements. A breakdown of the Alu subtypes showed that most changes occur within more recently evolved and active Alu subtypes Y and S (combined 1944 sites), compared with older, less active AluJ sites (FIG. 5D).

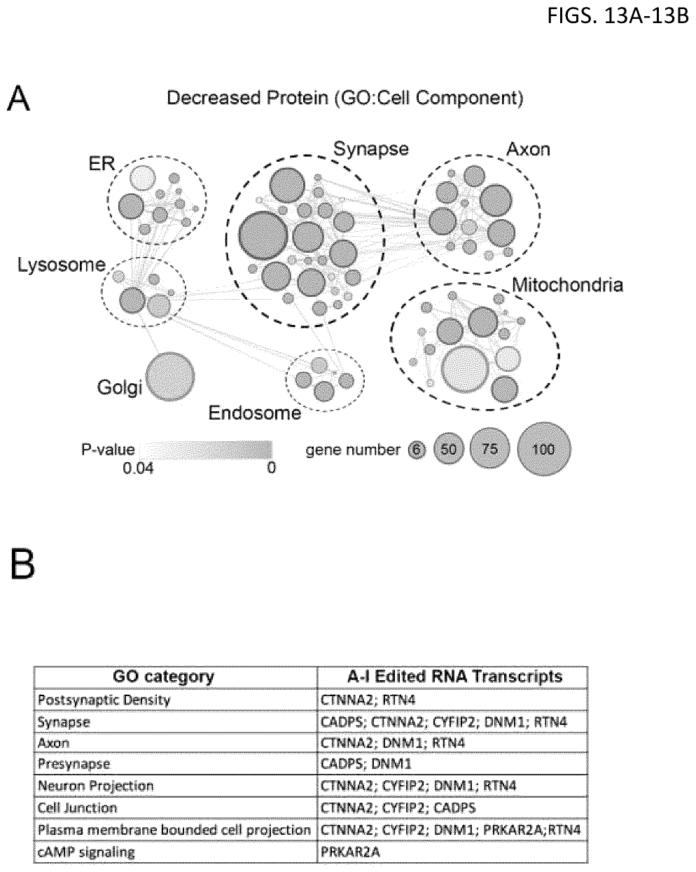

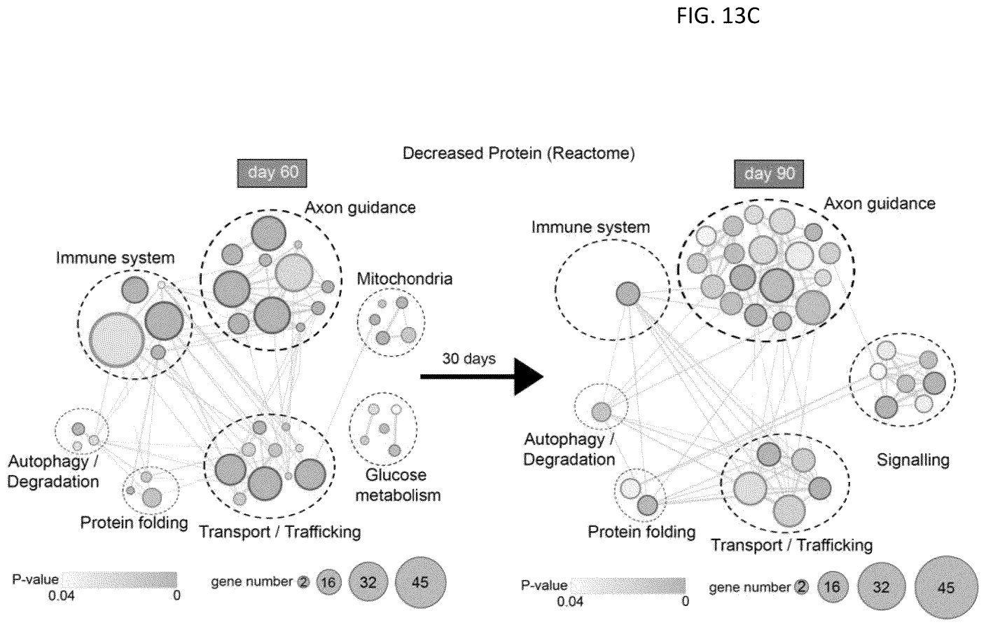

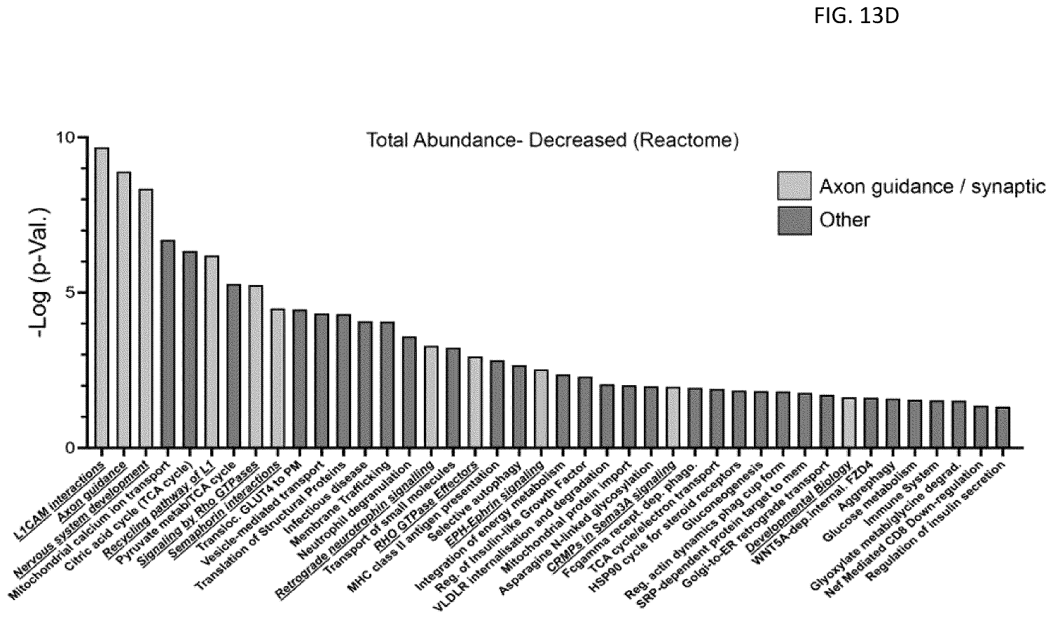

To determine the biological consequences of increased RNA editing, we performed network analysis on transcripts with increased A-to-I editing that are greater than 10% higher in A53T compared to corrected iPSn, and correlated the results with proteomic data. The largest proportion of transcripts with increased editing in PD occurred within the synapse, axon/axon guidance, mitochondria, and nucleus categories (FIG. 5E). These categories are consistent with known sites/transcripts identified from previous studies of physiological editing in human brain^{48,49,53,92}. Transcript networks with increased editing showed significant overlap with protein networks that are reduced in PD iPSn, including synapse, axon guidance and nervous system development (FIG. 13A-C). Prioritizing functional categories of reduced proteins by p-value revealed that axon guidance/nervous system development categories were the most significantly affected proteomic categories (FIG. 13D). Next, we correlated edited transcripts with their corresponding protein levels from SILAC-MS analysis. This revealed an overall negative correlation mostly driven by axon, synapse, and mitochondrial categories, (FIG. 5F, yellow and red plots). In this analysis, we included both CYFIP2 and CADPS transcripts found to be edited by Sanger sequencing (FIG. 4E-G). The overall negative correlation of A-to-I editing with protein expression suggests that the biological consequence of dysregulated editing may be to aberrantly reduce expression of the synapse, axon, and mitochondrial proteins that are essential for neuron survival.

Increased A-to-I Editing is Associated with Nuclear Retention of mRNA and Reduced Expression of Essential Axon and Synaptic Transcripts.

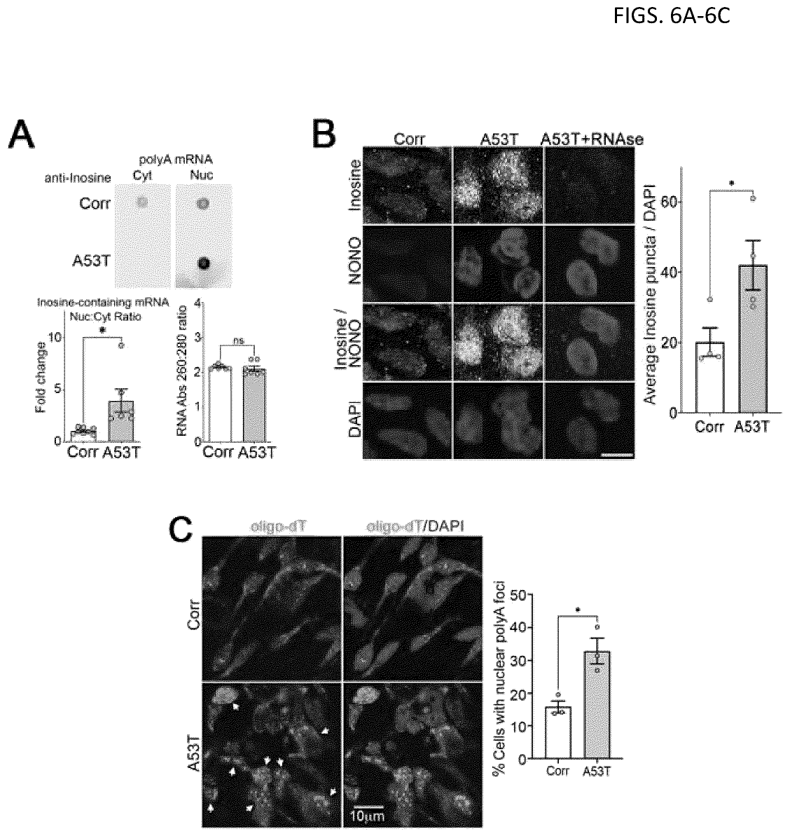

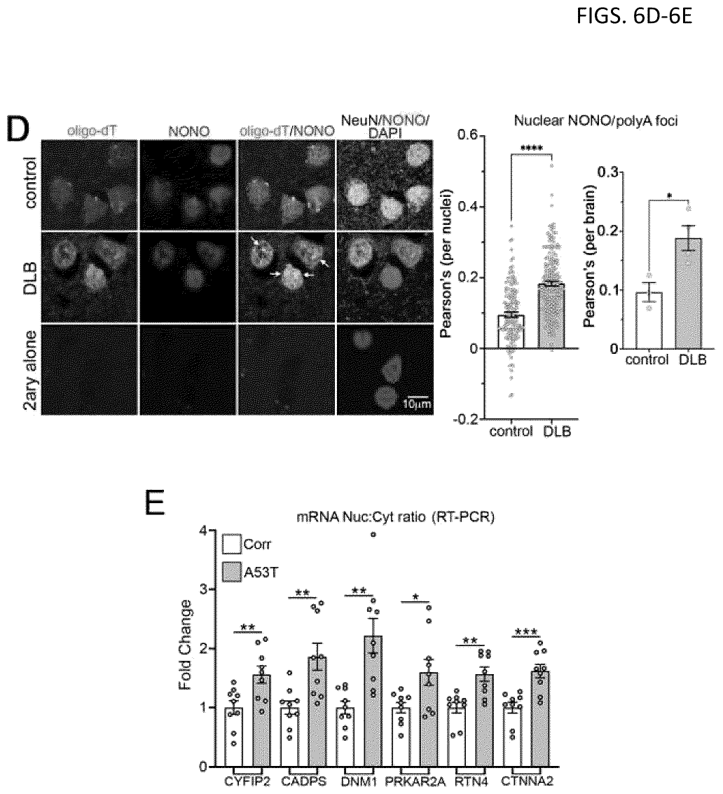

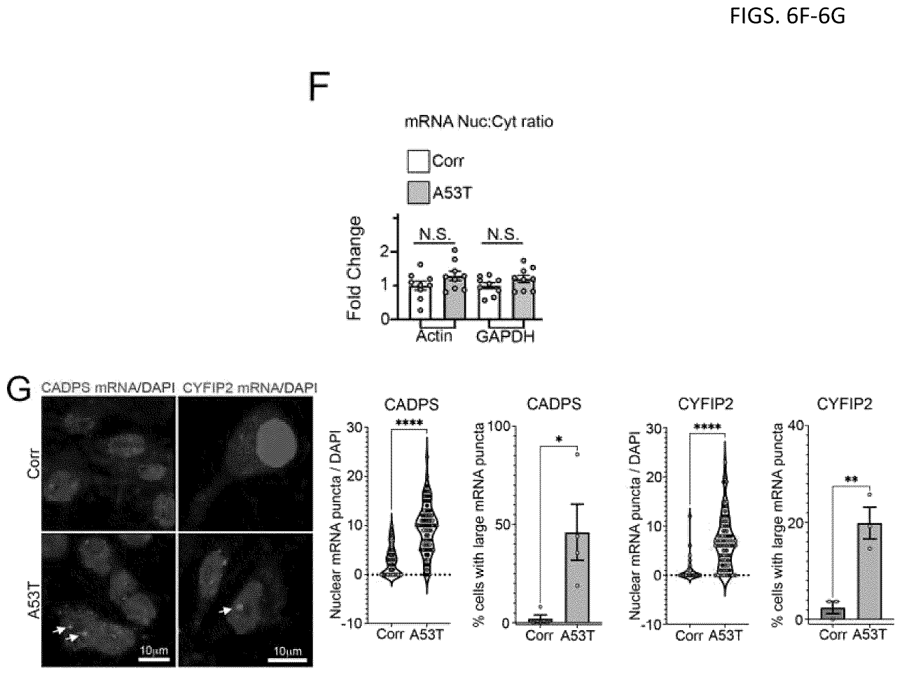

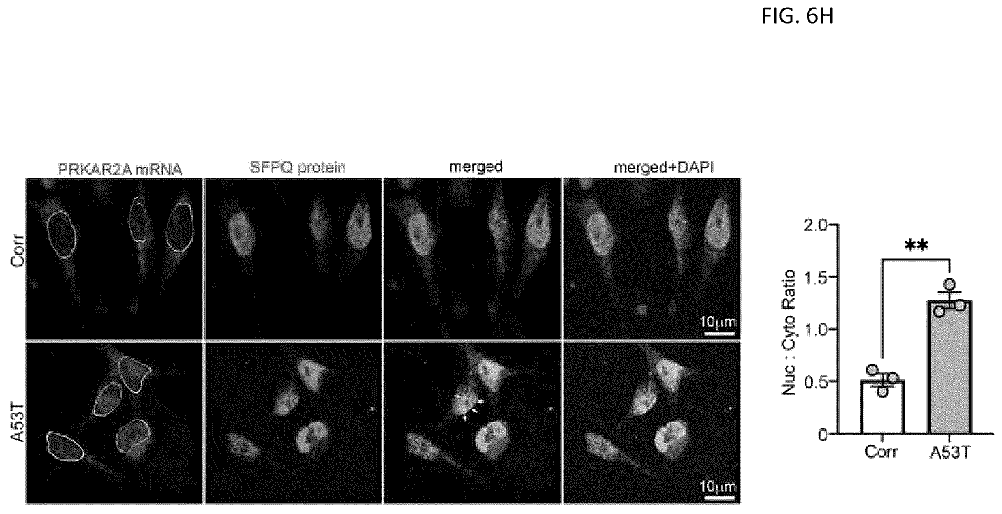

A-to-I editing may regulate gene expression in multiple ways³⁴. Previous studies showed that NONO/SFPQ complexes directly and preferentially bind inosine-containing RNA²⁹ and promote nuclear retention^{29,37-39,86}, and we observed that NONO/SFPQ inclusions accumulate in the nucleus of PD iPSn and DLB brain (FIGS. 1, 2). Therefore, we hypothesized that increased A-to-I editing results in nuclear sequestration and reduced expression of genes encoding for vital neuronal transcripts. To test this, we first determined the subcellular location of inosine-containing RNA. Isolation of poly-A mRNA from nuclear and cytoplasmic fractions followed by quantification of inosine showed an elevation of edited mRNA PD iPSn nuclei compared to isogenic controls, while almost no edited mRNA was found in the cytoplasm of PD iPSn (FIG. 6A). In comparison, isogenic corrected lines showed ˜65% inosine-mRNA in the nucleus and ˜35% in the cytoplasm. This indicates that physiologically edited mRNAs in healthy cells that lack NONO/SFPQ nuclear inclusions are exported to the cytoplasm. However under pathological conditions where increased A-to-I editing and NONO/SFPQ inclusions co-exist, inosine-containing mRNAs accumulate in the nucleus.

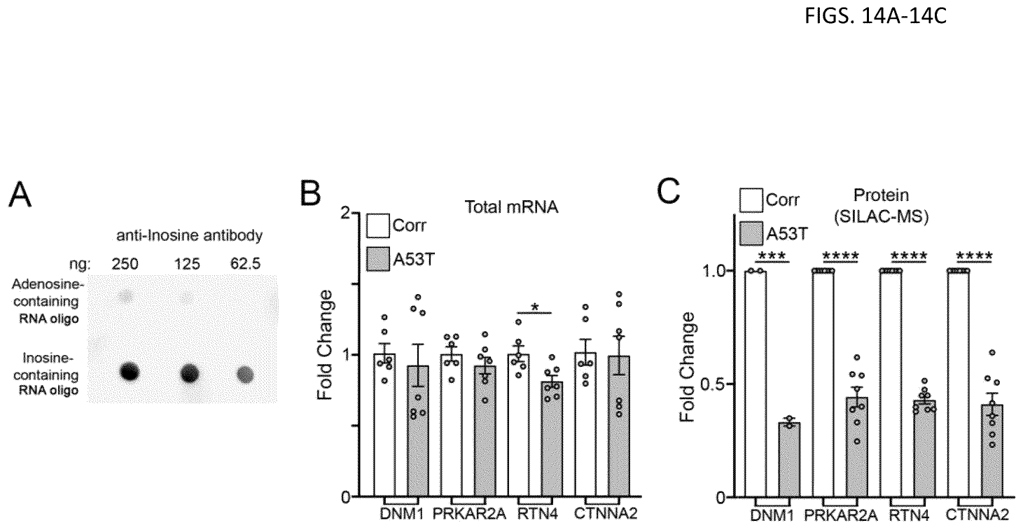

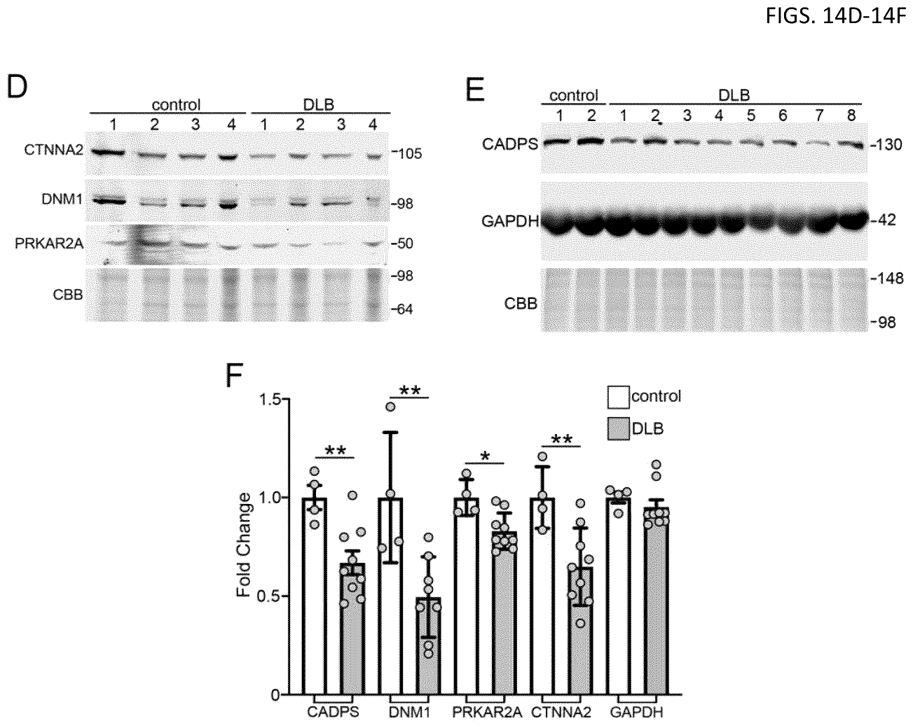

We confirmed elevated A-to-I edited RNAs in fixed PD cultures, which showed colocalized inosine and NONO puncta in the nucleus (FIG. 6B). The inosine signal was abolished by treatment with RNAse, indicating that it was derived from RNA (FIG. 6B), and anti-inosine antibody specificity was confirmed by dot blot analysis (FIG. 14A). Nuclear mRNA retention in PD cultures was supported by fluorescence in situ hybridization (FISH) using an oligo-dT probe in vitro and in vivo. FISH showed that PD iPSn accumulated mRNA in the nucleus, while most of the mRNA was found in the cytoplasm of isogenic corrected lines (FIG. 6C). In DLB patient brain, mRNA also accumulated in the nucleus of neurons, and colocalized with NONO inclusions (FIG. 6D). We next examined the mRNA localization of specific transcripts of the synaptic/axon/neuron projection categories with no or minimal change in total mRNA but with reduced protein levels in PD iPSn including CYFIP2, CADPS, DNM1, PRKAR2A, RTN4, and CTNNA2 (FIG. 4E, 13B, 14B, 14C). Fractionation followed by mRNA purification and quantification by RT-PCR revealed that all transcripts examined were elevated in the nucleus compared to isogenic controls (FIG. 6E). Analysis of mRNA transcripts that are not known to be extensively edited, including GAPDH and actin, had similar nuclear/cytoplasmic ratios (FIG. 6F). Together, this supports the conclusion that nuclear mRNA sequestration results in reduced expression of synaptic and axonal genes.

We validated the localization changes of select mRNAs by FISH in fixed PD iPSn. Using probes against CYFIP2 and CADPS mRNA, we found unusually large (1-2 mm in diameter) mRNA inclusions in the nucleus of PD iPSn and depletion of cytosolic mRNA (FIG. 6G, white arrows). Analysis of PRKAR2A mRNA also confirmed increased nuclear localization in PD iPSn, and co-staining with SFPQ revealed puncta that co-localized with PRKAR2A mRNA in the nucleus (FIG. 6H). We validated the proteomic data for each of these targets, showing that each protein was significantly decreased in PD iPSn by western blot analysis of iPSC culture batches that differed from those used for proteomic analysis (FIG. 6I). Control proteins including GAPDH and actin were not statistically different (FIG. 6I). Several of the axon/synaptic proteins were also decreased in DLB brain (FIG. 14D-F). These data suggest that aberrant nuclear mRNA retention of edited mRNAs may be responsible for the decreased protein expression of axon and synaptic proteins prior to neurodegeneration.

### Reducing RNA Editing Restores the Expression of Axonal and Synaptic Proteins.

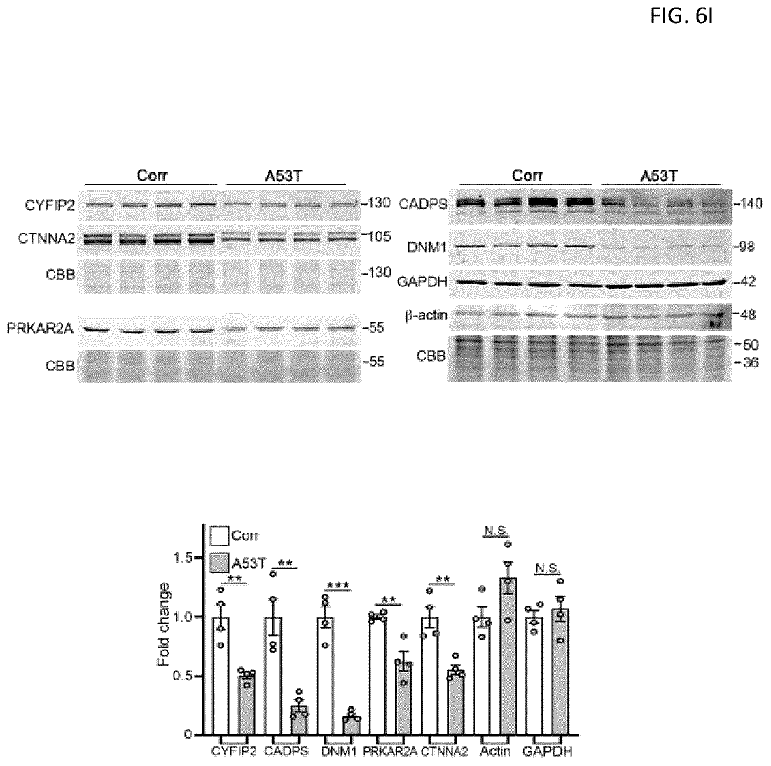

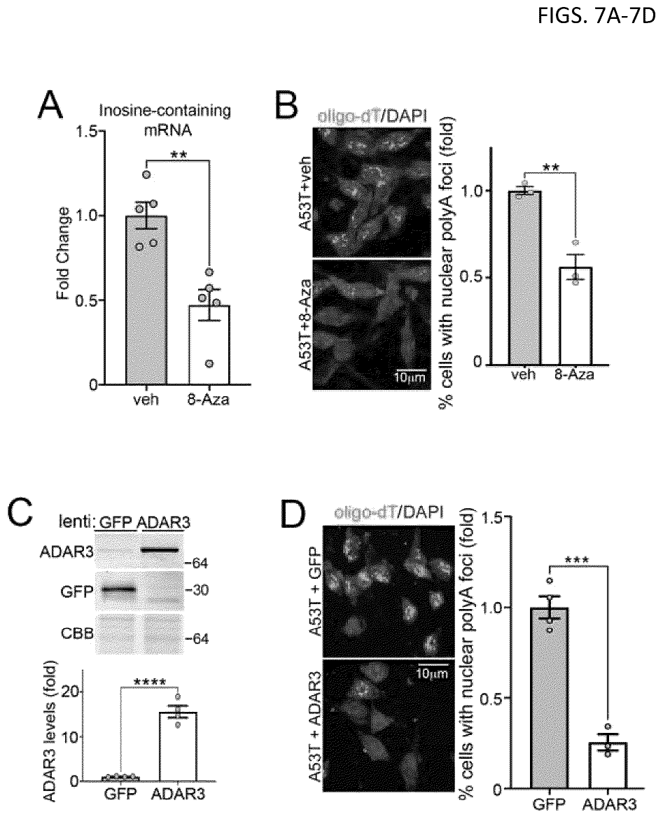

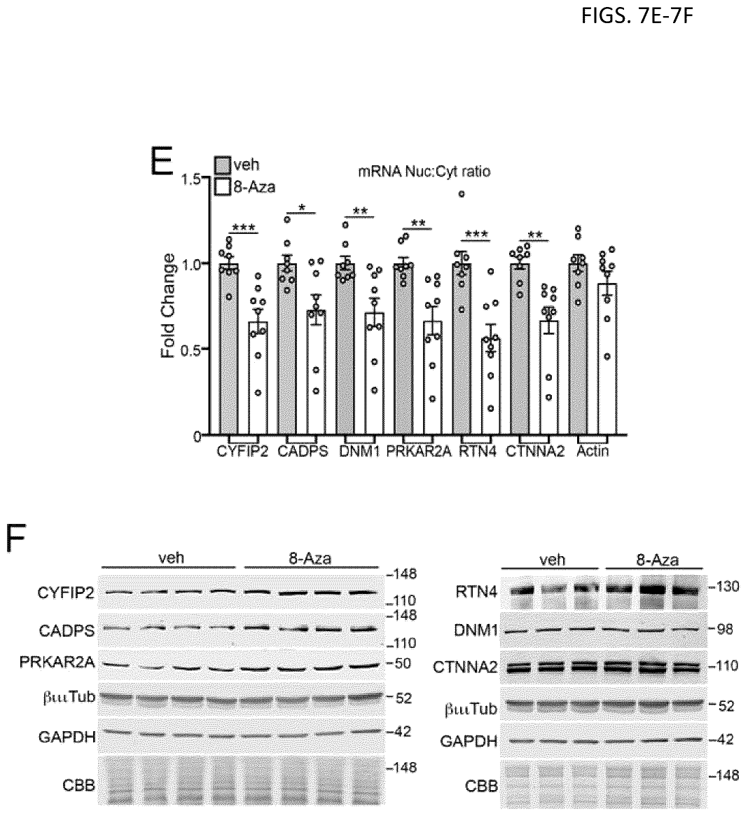

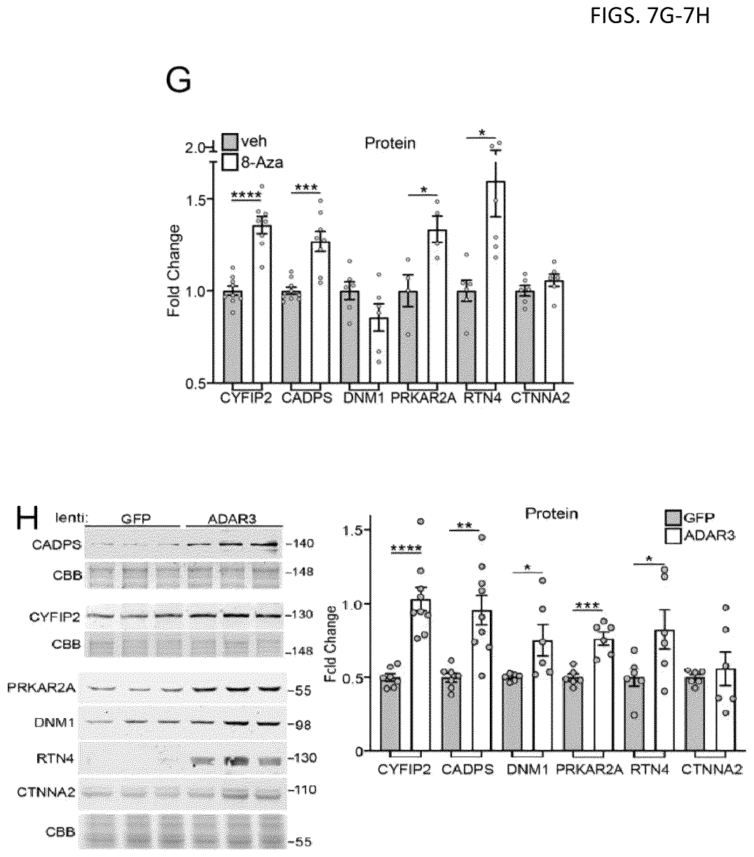

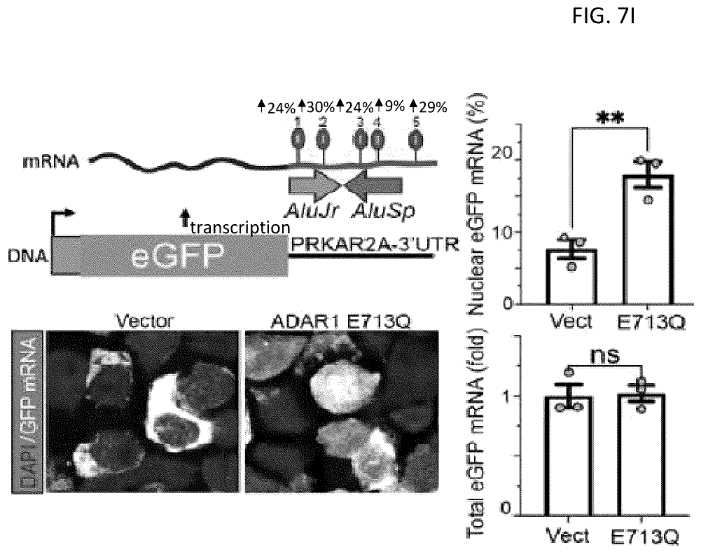

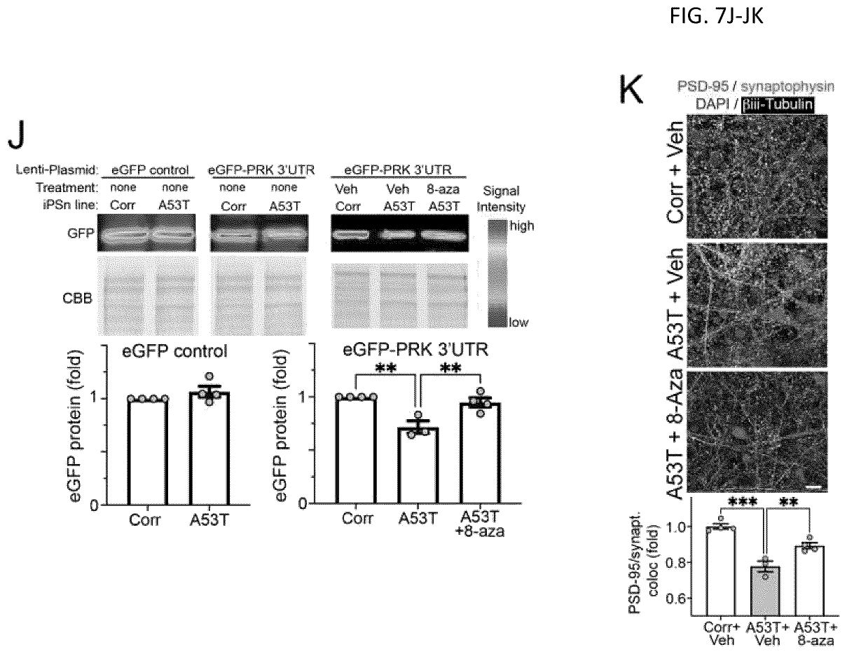

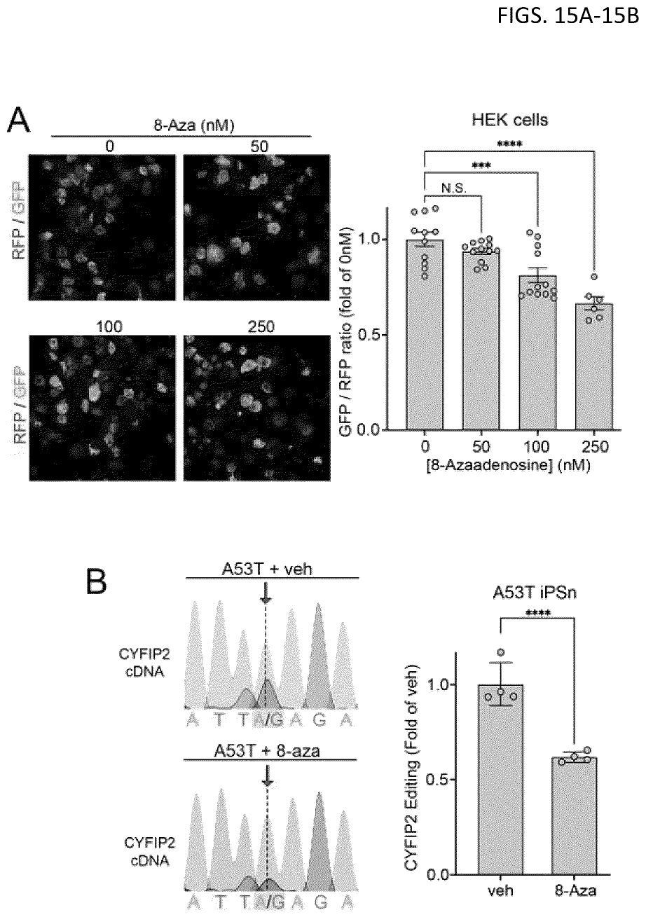

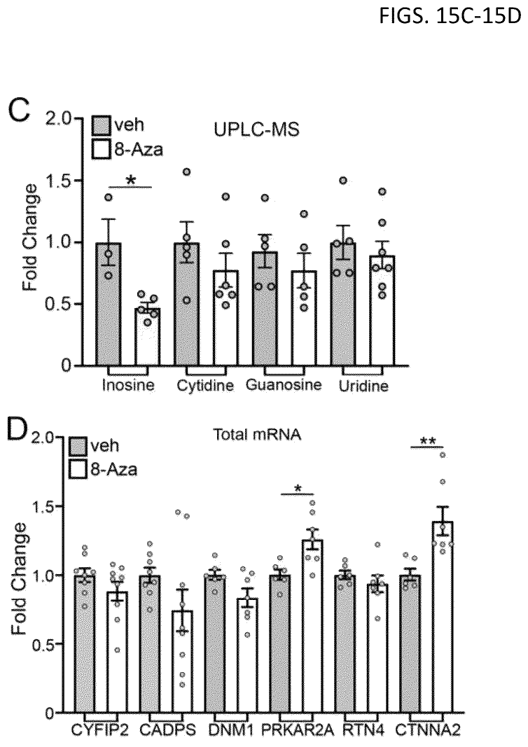

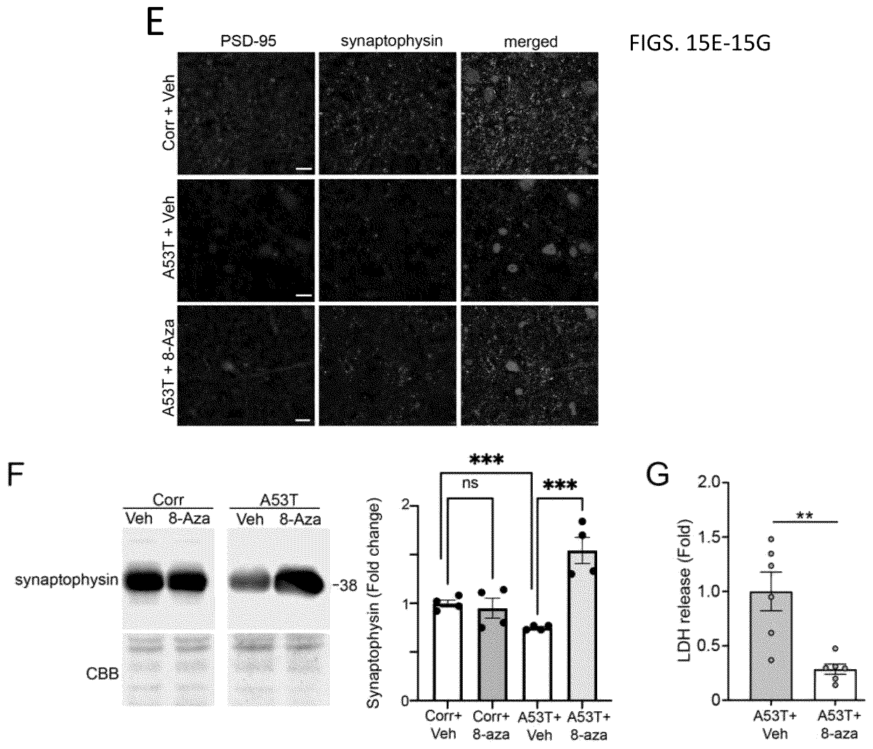

To determine if nuclear mRNA retention and reduced expression occurs through increased A-to-I editing, we reduced inosine-mRNA in PD iPSn by both pharmacological and genetic approaches. We first treated iPSn with an ADAR1 inhibitor, 8-azaadenosine (8-aza). As an analogue of its naturally occurring substrate, 8-aza-containing RNA binds with greater affinity to the ADAR active site, indicating that it can act as a competitive inhibitor⁹³. Free 8-aza can be incorporated into polynucleotides including RNA through its triphosphate⁹⁴⁻⁹⁶, and multiple studies showed that free 8-aza, at concentrations as low as 100 nM, can reduce A-to-I editing and hence increase dsRNA structure when added to cell cultures^{47,97-98}. However, another study concluded that 8-aza had no effect on A-to-I editing in breast cancer cell lines when it was used for the purpose of killing cancerous cells at cytotoxic doses of 1-to-10 uM⁹⁹. Therefore, we first determined if 8-aza could inhibit A-to-I editing in our culture systems without inducing toxicity. Using HEK cells and a previously established A-to-I editing reporter¹⁰⁰, we found that editing was inhibited between 100 and 250 nM of 8-aza (FIG. 15A), and concentrations above 1 uM were toxic. Assessment of an endogenous ADAR target (CYFIP2 mRNA) showed 8-aza reduced editing by 40% in PD iPSn, which is similar to levels found in isogenic corrected lines (FIG. 15B). Next, we assessed the global levels of A-to-I editing by purifying polyA mRNA from the nucleus of vehicle and 8-aza treated PD iPSn. Inosine was detected in these samples by both immuno-dot blot and HPLC-MS. Both detection methods showed a ˜50% reduction in inosine while the quantity of other nucleotides within the polyA mRNA were not changed (FIG. 7A; FIG. 15C).

Having established a concentration of 8-aza that could reduce A-to-I editing in the absence of toxicity, we next analyzed mRNA localization by polyA-FISH. 8-aza reduced nuclear mRNA punctated inclusions in PD iPSn by ˜50% compared to vehicle treated cultures (FIG. 7B). We confirmed this effect by overexpressing ADAR3 through lenti-viral transduction (FIG. 7C). ADAR3 also reduced nuclear polyA puncta by 75% (FIG. 7D), confirming that the rescue effect was due to reduced A-to-I editing. Next, we assessed the nuclear: cytosolic mRNA ratios of specific axon/synaptic transcripts after 8-aza treatment and found that they were decreased, reflecting more efficient export to the cytoplasm (FIG. 7E). Quantification of total mRNA showed a slight increase in PRKAR2A and CTNNA2, while no changes were found in any other transcripts, indicating that the main effect of reduced A-to-I editing was to restore nuclear: cytosolic ratios of these particular transcripts (FIG. 15D). Consistent with enhancing nuclear mRNA export, protein expression of CYFIP2, CADPS, PRKAR2A, and RTN4 was restored to control levels by 8-aza (FIG. 7F, G). We confirmed increased expression of these proteins in ADAR3 lenti-transduced PD iPSn (FIG. 7H). The protein levels of CTNNA2 were not improved by either 8-aza or ADAR3, while DNM1 protein was only rescued by ADAR3 (FIG. 7 F-H). This could indicate a more effective rescue by ADAR3 compared to 8-aza, or that additional mechanisms are required to fully restore their expression. These data indicate that nuclear retention in PD iPSn is rescued by reducing A-to-I editing.

We next directly tested the role of A-to-I hyper-editing in nuclear retention and protein expression by utilizing a reporter plasmid expressing GFP fused to the editing-enriched region within the 3′UTR of PRKAR2A. This region contains 2 human-specific inverted repeat (IR) Alu regions (AluJr and AluSp) and a cluster of 5 sites that are hyper-edited in PD iPSn (FIG. 7I). Increased editing was induced in HEK cells by expression of a hyperactive form of ADAR1 (E713Q). Expression of the reporter plasmid (GFP-PRK-3′UTR) in ADAR1 hyperactive cells resulted in nuclear retention of GFP mRNA compared to cells expressing endogenous wild-type ADAR1, while total GFP mRNA was not changed (FIG. 7I). Since the double-stranded (ds) RNA IRAlu elements are expressed equally in both vector and ADAR E713Q cells (FIG. 7I bottom graph), the effect on nuclear retention is likely due to hyper-editing as opposed to other structural characteristics of the IRAlus themselves. We confirmed this effect in PD iPSn. Expression of the GFP-PRK-3′UTR construct revealed lower GFP protein expression in PD lines compared to isogenic corrected lines (FIG. 7J). This decline was a direct result of the PRKAR2A 3′UTR, since expression of control GFP plasmids using the same promoter but lacking the 3′UTR were not different between corrected and patient iPSn (FIG. 7J). Treatment with 8-aza improved GFP expression in PD iPSn, indicating that reduced protein expression occurred from A-to-I hyper-editing (FIG. 7J). These data further support the hypothesis that increased A-to-I editing in PD iPSn contributes to nuclear retention and reduced expression of axon/synaptic proteins.

We next determined the effects of reducing A-to-I editing on neuronal health. Since PD iPSn begin to show neurite degeneration after day 100^{24,63}, cultures were treated with 8-aza between day 90-120 in an attempt to prevent degeneration. At day 120, synapses were quantified by immunofluorescence staining of fixed cultures by colocalization analysis of post-synaptic (PSD-95) and pre-synaptic (synaptophysin) markers (FIG. 7K, FIG. 15E). We found a decline in the number of colocalized puncta in PD iPSn compared to isogenic controls (FIG. 7K, FIG. 15E). Both the staining intensity and colocalization of PSD-95 and synaptophysin were reduced in PD iPSn, while b-iii tubulin was not dramatically affected (FIG. 7K, FIG. 15E). 8-aza treatment at 200 nM for one month increased the levels of individual synaptic markers and improved their colocalization, suggesting improved synapse formation (FIG. 7K, FIG. 15E). Confirmation by western blot showed that 8-aza rescued synaptophysin levels in PD iPSn (FIG. 15G). Importantly, no change was seen when isogenic corrected lines were treated with 8-aza, suggesting that the treatment is not toxic to control iPSn (FIG. 15F). We used a third assay to assess cell health by measuring the release of lactose dehydrogenase (LDH) from dying neurons. Inhibiting A-to-I editing also reduced LDH release, further supporting that neuronal health was improved (FIG. 15G).

### Increased RNA Editing Contributes to Pathological Aggregation of NONO/SFPQ.

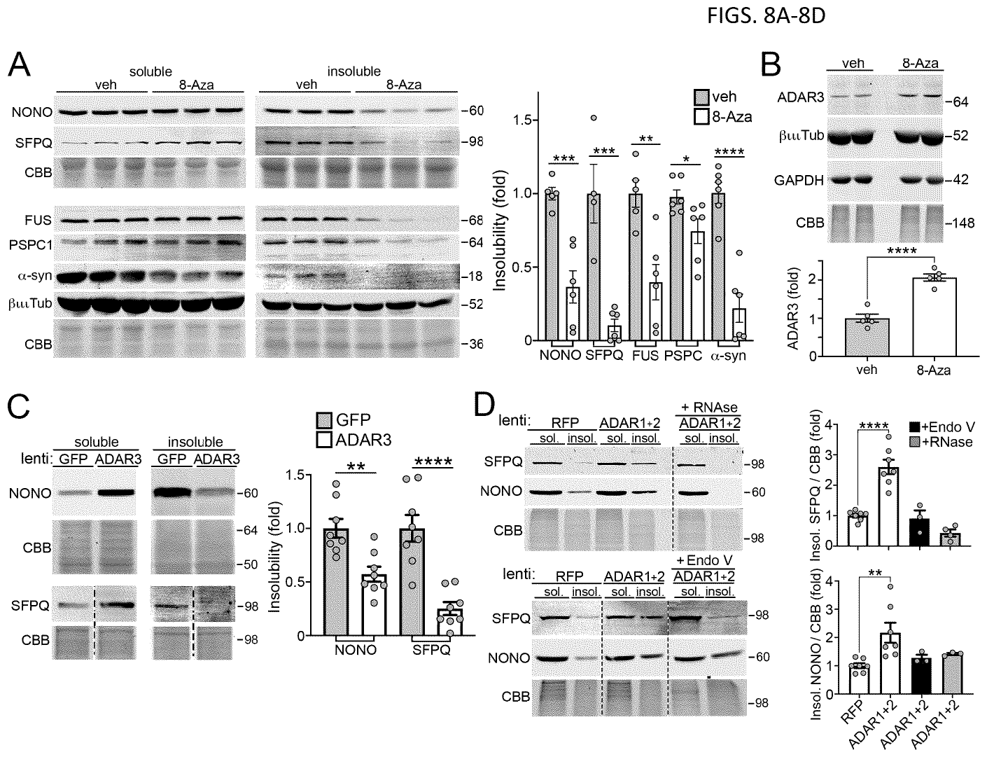

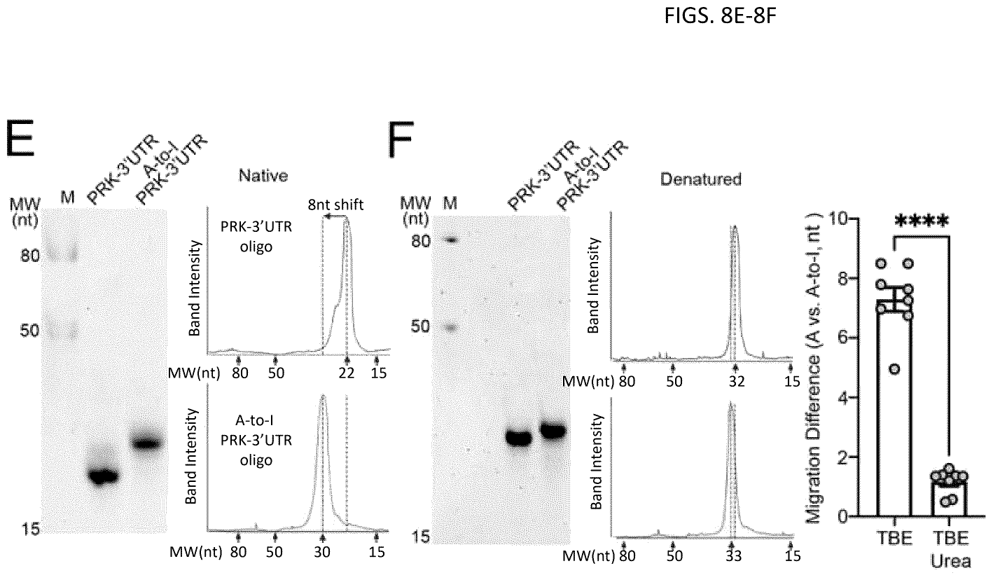

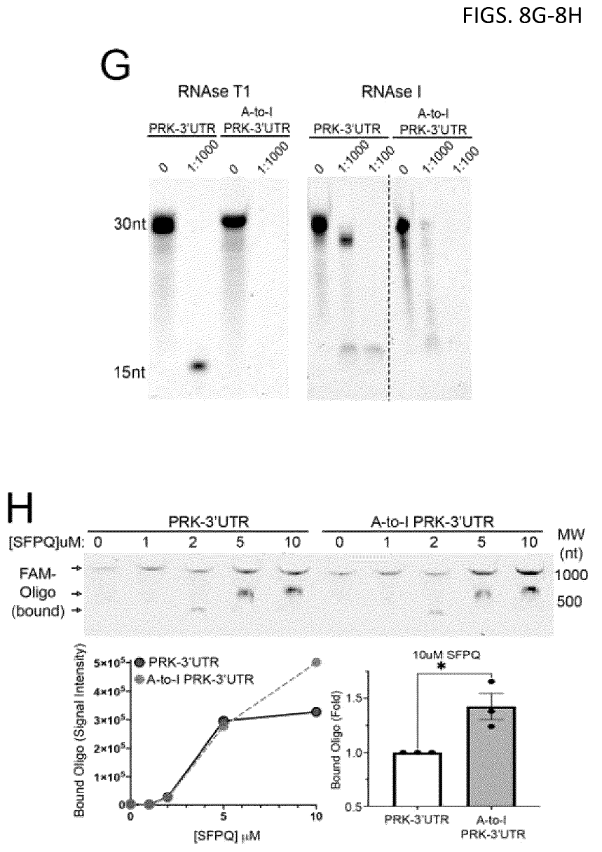

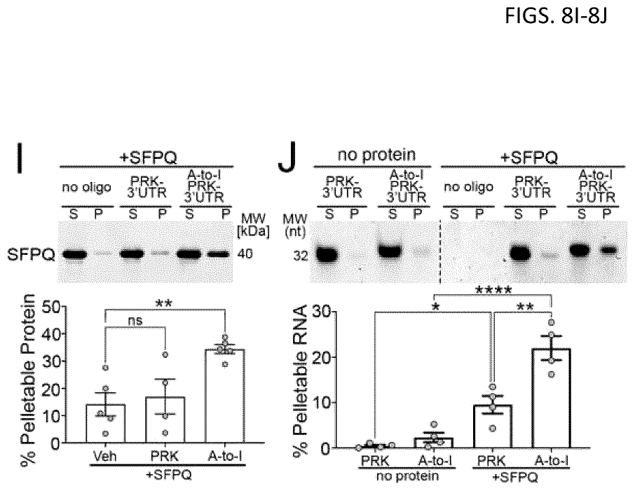

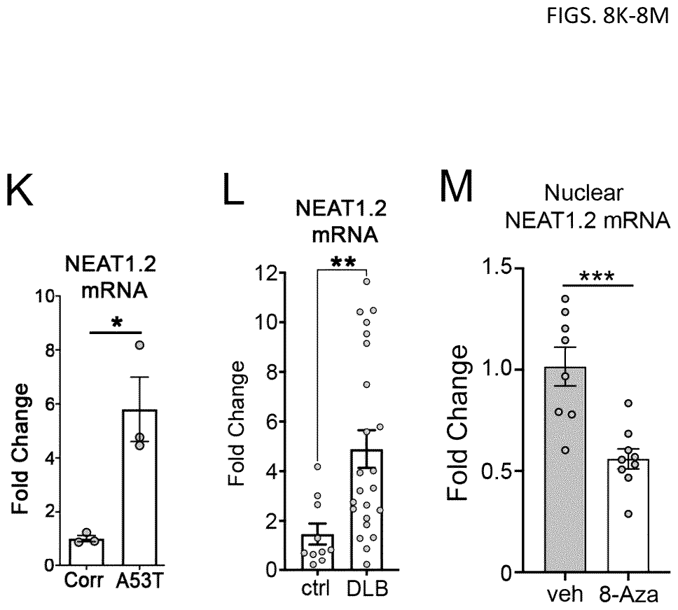

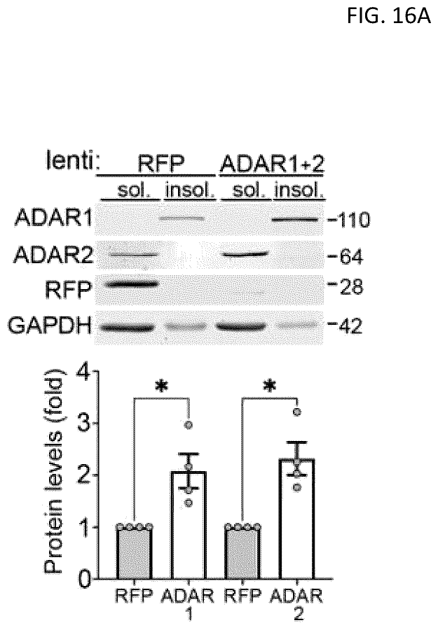

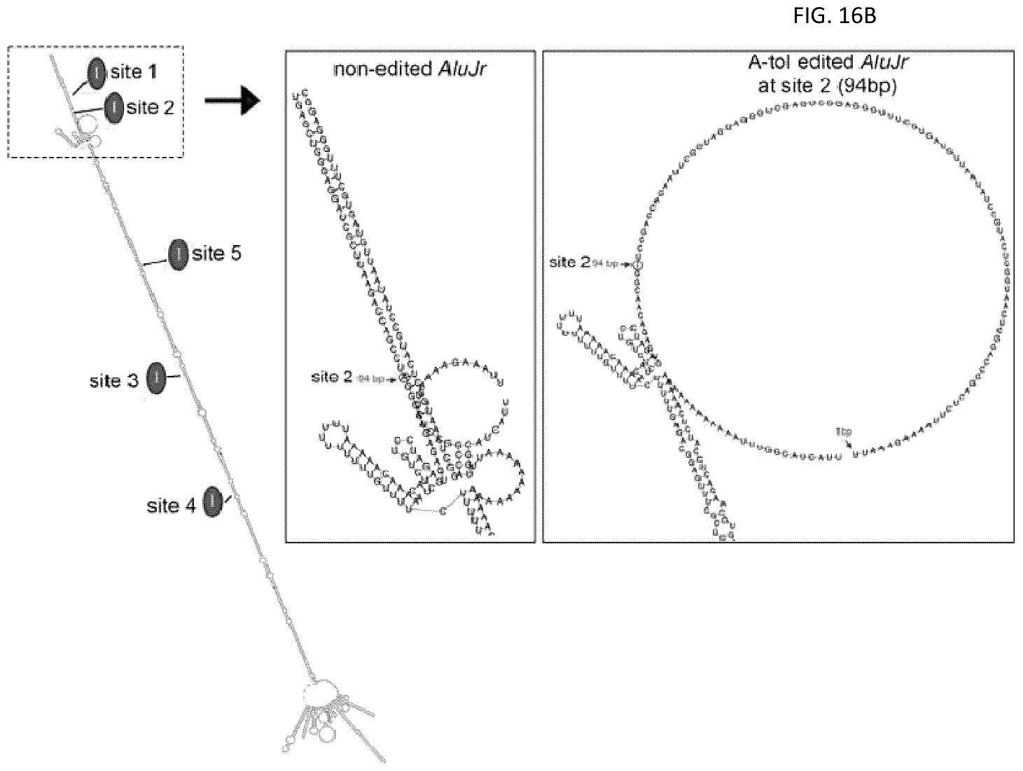

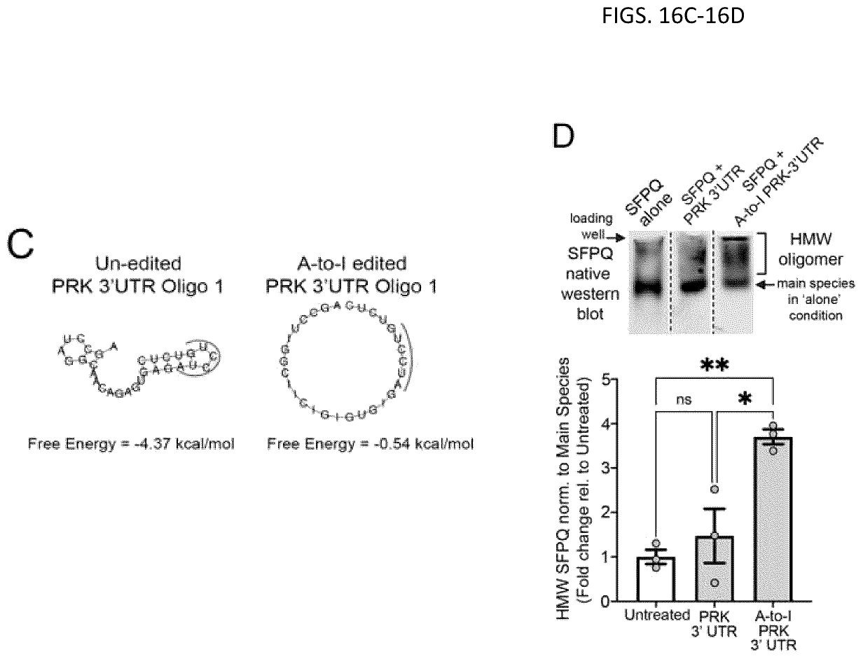

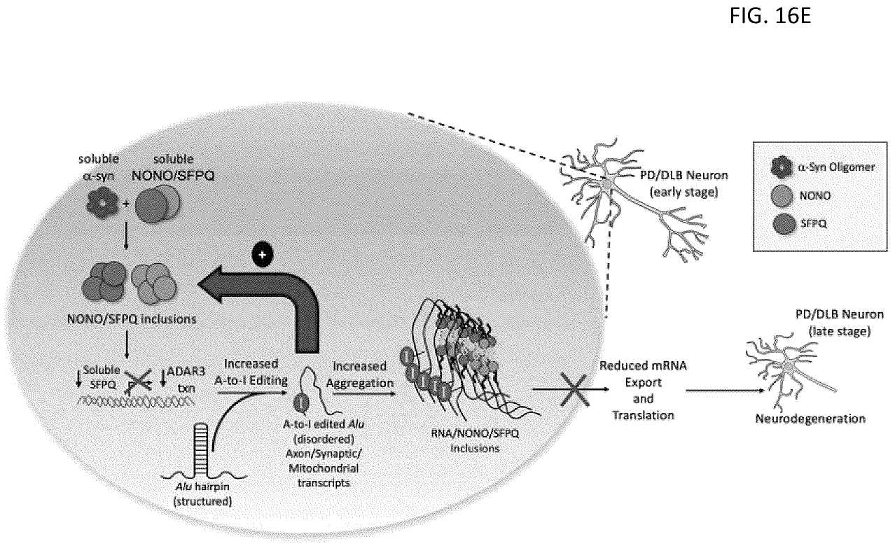

We next determined if A-to-I edited RNAs play a role in nuclear protein aggregation. One month 8-aza treatment reduced insoluble NONO, SFPQ, FUS, PSPC1, and eliminated insoluble α-syn in PD iPSn (FIG. 8A). Control proteins including b-iii-tubulin and GAPDH were not changed (FIG. 8A, B). The increase of soluble SFPQ was sufficient to restore the expression of ADAR3 (FIG. 8B), suggesting that the physiological function of SFPQ was restored. We then directly overexpressed ADAR3 in PD iPSn, which also reduced insoluble NONO and SFPQ by 50% and 75% respectively and is similar to physiological levels (FIG. 8C). We further examined the mechanistic relationship between increased editing and NONO/SFPQ aggregation by overexpressing hyperactive forms of ADAR1 and 2¹⁰¹ in healthy control iPSn using lentivirus, which resulted in ˜2 fold overexpression (FIG. 16A). At 14 days post infection (dpi), insoluble SFPQ and NONO were elevated by hyperactive ADARs compared to RFP infected controls (FIG. 8D). Removal of edited RNA from the lysate with either RNAse, or Endonuclease V that selective degrades inosine-containing nucleic acid, reduced insoluble NONO/SFPQ aggregates (FIG. 8D). Collectively, these data indicate that increased RNA editing promotes insoluble NONO/SFPQ aggregates in midbrain cultures. This suggests that once increased editing is triggered, inosine-RNAs can further exacerbate or stabilize insoluble NONO/SFPQ aggregates, participating in a self-propagating pathological cycle.

The mechanism of how edited RNA influences protein aggregation was examined in further detail in vitro. In silico structural analysis of the 1 kb edited-enriched region of the PRKAR2A 3′ UTR using RNAfold revealed the formation of two double-stranded RNA structures formed from AluJr and AluSp. Given that this region is highly edited in iPSn, and ADAR1 can only modify dsRNA substrates, these dsRNA structures likely occur in vivo. We found that A-to-I conversion at site 2 that normally participates in A:U base pairing (FIG. 16B) disrupted the stem structure of AluJr, converting the stem structure to an open single-stranded loop (FIG. 16B, right). Conversion to inosine at the other 4 sites showed no structural changes by RNAfold, possibly because these edits are mostly within bulges and do not participate in base pairing of the dsRNA. We generated a 30 bp RNA oligonucleotide that contains site 2 to model the full length 3′UTR of PRKAR2A. Similar to the full-length 3′UTR, this adenosine-containing oligo is predicted to form a structured hairpin, while replacing A with I induces structural disorder (FIG. 16C). The oligo also contains a potential SFPQ binding site (AUCCUG)¹⁰². Structural analysis of the RNA oligo by native gel electrophoresis showed that the adenosine-RNA oligo migrated faster than predicted, at 22 nucleotides (nt), suggesting the formation of secondary structure (FIG. 8E). The inosine-RNA oligo migrated at the expected size of 30 nt (FIG. 8E). Denaturing the oligos with urea shifted the weight of the adenosine-RNA oligo to the expected size of ˜30 bp, while denaturation had essentially no effect on the inosine-RNA oligo (FIG. 8F). If the adenosine oligo is double-stranded, it should exhibit increased resistance to RNAse treatment, while ssRNA will be completely degraded. We found that treatment with either RNAse-T1 or RNAse-I completely digested the inosine-RNA oligo indicating it has the characteristics of a ssRNA in solution (FIG. 8G). The adenosine-RNA oligo was partially resistant, suggesting that it contains double-stranded structure (FIG. 8G). These data indicate that converting A-to-I triggers structural changes in the AluJr region of the PRKAR2A 3′UTR mRNA.

To directly test the effect of A-to-I edited RNA oligos on SFPQ, they were incubated with recombinant SFPQ followed by measurement of binding and aggregation. Binding was assessed by gel shift analysis, holding a FAM-labeled oligo at 100 nM and varying the concentration of SFPQ. We found that both oligos bound to SFPQ equally at protein concentrations of 1, 2, and 5 uM. However, when SFPQ was increased to 10 uM, inosine-RNA oligos bound ˜40% more compared to adenosine-RNA oligos (FIG. 8H). Furthermore, inosine-RNA oligos directly potentiated SFPQ aggregation in vitro, demonstrated by sedimentation/denaturing western blot analysis (FIG. 8I). RNA analysis by denaturing PAGE of the same samples showed that while the RNA oligos alone were in the supernatant fraction, the inosine-RNA oligo accumulated in the pellet fraction when mixed with SFPQ, indicating co-aggregation with SFPQ (FIG. 8J). We confirmed that inosine-RNA oligos potentiate SFPQ aggregation by a distinct native western blot assay, showing increased HMW SFPQ aggregates (FIG. 16D). Together, these data indicate that A-to-I editing promotes binding and potentiates aggregation of SFPQ directly in vitro, and is consistent with culture experiments (FIG. 8D). Increased binding of inosine-containing ssRNA to SFPQ provides a mechanistic explanation for the nuclear retention and reduced gene expression observed under conditions of elevated editing and NONO/SFPQ nuclear inclusions (FIG. 6).

Finally, we measured the levels of NEAT1__2 long non-coding RNA in patient material. This is because NEAT1_2 is required for paraspeckle formation and forms a foundation for NONO and SFPQ aggregates to form. We found that NEAT1_2 RNA levels were dramatically upregulated in both PD iPSn and DLB patient brain (FIG. 8 K, L). Furthermore, reducing RNA editing with 8-aza reduced NEAT1_2 levels by ˜50% (FIG. 8M). These data indicate that NEAT1_2 may also be involved in the pathogenesis of PD and DLB, and that reducing RNA editing alone is sufficient to reduce NEAT1_2. Therapies that target NEAT1_2 levels may also provide therapeutic benefit by helping to dissolve nuclear aggregates of NONO and SFPQ.

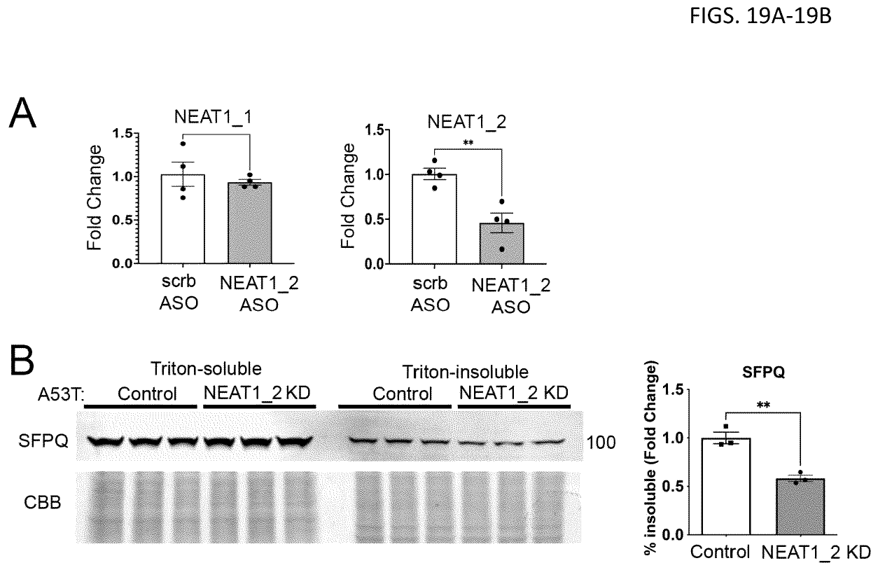

We developed an anti-sense oligonucleotide (ASO) gapmer (short DNA fragment flanked by RNA mimics) that specifically targets NEAT1_2 at the 3′ end of the transcript, while leaving NEAT1_1 levels unchanged, to be utilized in vitro and in vivo. Binding of the gapmer to NEAT1_2 reduces transcript levels through RNAse H degradation. The gapmer sequence includes 2-O-methoxyethyl (2′MOE) modifications in the RNA mimetic regions to enhance stability to nucleases in vivo and is as follows: 5′-2MOErA/i2MOErG/i2MOErA/i2MOErA/i2MOErA/CACCTGCGGC/i2MOErG/i2MOErG/i2MOErC/i2MOErT/i2MOErC (SEQ ID NO: 5). Transfection of the ASO gapmer in PD A53T midbrain neurons reduced NEAT1_2 levels by 50% (FIG. 19A). Next, we tested if NEAT1_2 levels contribute to pathological SFPQ aggregation in PD midbrain neurons. We found that knock-down of NEAT1_2 for 2 weeks reduced insoluble SFPQ by 50% (FIG. 19B). These data demonstrate that NEAT1_2 contributes to pathological aggregation and that reduction by ASOs may be a viable therapeutic option for neurodegenerative disorders and other diseases characterized by pathological SFPQ aggregation.

### Discussion

We examined how proteome composition changes in synucleinopathy patient material by focusing on solubility shifts defined by a concomitant decrease in soluble and increase in insoluble proteins. We discovered aberrant aggregation of predominantly RNA binding proteins in the nucleus, NONO and SFPQ. Importantly, analysis of human idiopathic DLB brain showed NONO and SFPQ aggregates in all samples analyzed and, in some cases, more abundant than insoluble α-syn, indicating its importance for sporadic synucleinopathies. Studies have identified other aggregation-prone proteins in disease beyond α-syn, amyloid-beta, tau, and TDP-43 that are commonly used to define various neurodegenerative diseases, including TMEM106B¹⁰³⁻¹⁰⁶, although the pathophysiological role of TMEM106B aggregates are not clear given that they are also found in healthy control brains. The specific toxic function of Lewy inclusions has also been debated for several decades and is not completely understood. Our data shows a clear mechanistic connection between NONO/SFPQ inclusions and toxicity through nuclear retention of A-to-I edited transcripts that are vital for neuron survival. Besides the fact that NONO/SFPQ are established RNA binding proteins, previously established connections between NONO/SFPQ complexes and nuclear retention of inosine-containing transcripts led us to focus on this pathway as a potential explanation for reduced protein expression and axon/synaptic degeneration^{29,37-39,86}, Rescue experiments showed that reducing A-to-I editing alone, through either 8-aza or ADAR3, is sufficient to rescue synaptic loss and dissolve pathological aggregates in patient-derived cultures, suggesting that editing plays an important role in the neurodegenerative process. Similarly, expression of ADAR1/2 hyperactive mutants in healthy control iPSn induced NONO/SFPQ aggregation, indicating that increased A-to-I editing contributes to inclusion formation in the nucleus of PD iPSn. These studies indicate that in addition to reducing the expression of vital neuronal proteins, A-to-I edited transcripts actively play a role in potentiating protein aggregation and disrupting nuclear function (FIG. 16E). Neurodegenerative diseases are classified as proteinopathies based on the aggregation of a single protein, while less attention is given to non-proteinaceous pathologies and their potential role in disease. Our studies support the notion that not one, but multiple proteins, accumulate in neurodegenerative diseases. Furthermore, non-protein containing pathologies including mRNA inclusions play an important role in pathogenesis.

Mechanistic studies showed that soluble α-syn oligomers directly influence the aggregation of purified recombinant SFPQ while insoluble α-syn fibrils (PFFs) have no effect. While SFPQ alone has an inherent ability to form pelletable aggregates with mild ThT reactivity in vitro, the addition of α-syn oligomers initially slows this conversion in the first 24 hrs by kinetic stabilization of soluble SFPQ intermediates (FIG. 3H). At later stages (48 hr), these SFPQ intermediates were then incorporated into pelletable fractions with enhanced ThT reactivity, providing an explanation for the biochemical and histological findings of insoluble nuclear inclusions observed in iPSn and DLB patient brain. In PD iPSn, we found that α-syn colocalizes with NONO or SFPQ as either distinct puncta or in a discrete, diffuse pattern within the nucleus (FIG. 3 A, B, and FIG. 12). Most of the colocalized signal occurred in the nucleus, however a minor PLA signal of SFPQ-α-syn colocalized in the cytoplasm (FIG. 3A). In DLB post-mortem brain, α-syn/NONO/SFPQ was observed within the nuclear puncta but was completely devoid in cytoplasmic Lewy inclusions that are comprised mainly of fibrillar α-syn (FIG. 11F). This is consistent with in vitro experiments showing that α-syn fibrils had no influence on SFPQ aggregation, and suggest that soluble α-syn oligomers likely trigger structural changes in nuclear SFPQ leading to aberrant mRNA editing and retention in vivo.

In silico and in vitro studies showed that introduction of inosine in the 3′UTR of PRKAR2A altered the structure of the IRAluJr region (FIG. 16 B, C). This in turn, directly promoted binding and aggregation of recombinant SFPQ in vitro (FIG. 8H-J; FIG. 16D), which is consistent with our observations in iPSn cultures that overexpress hyperactive forms of ADARs (FIG. 8D). We showed that inosine-containing RNA oligos bound with greater affinity to SFPQ only at higher concentrations of 10 uM, which may be related to its higher propensity to polymerize at this concentration (FIG. 8H). Using a region of the PRKAR2A 3′UTR as an example, our data suggest that increased editing induces structural disorder and promotes RNA that behaves more like ssRNA, which then lead to higher SFPQ binding affinity. Normally, SFPQ can bind both ds and ssRNA^{33,102}, however it is possible that structural changes induced by A-to-I editing expose more high-affinity SFPQ binding sites in ssRNA. Nuclear retention is known to occur through hyper-editing of IRAlus, although it is possible that editing in non-Alu regions at even one site is sufficient to alter dsRNA to a structure that promotes SFPQ binding and nuclear retention. This is supported by the analysis of the 3′UTR of PRKAR2A which, although is hyper-edited, only requires one A-to-I change to induce a ssRNA loop (FIG. 16B). It has been long known that A-to-I editing alters dsRNA into an RNA with more single-stranded characteristics, including sensitivity to single-stranded RNAse¹⁰⁷ and altered migration on native PAGE gels³⁵. Previous work also showed that A-to-I editing promotes the interaction with RNA binding proteins through destabilizing dsRNA into an RNA that is more single-stranded in nature¹⁰⁸. These studies are consistent with the changes we observe in PRKAR2A mRNA, suggesting that the increased interaction with SFPQ occurs from ds to ssRNA conversion. Once bound, the edited RNA may serve as a scaffold that promotes the seeding and/or stabilization of insoluble SFPQ polymers (FIG. 16E), although more studies are required to determine the specific aggregation stage where RNAs act. Upon surpassing a threshold, pathological inclusions form and aberrantly anchor essential neuronal transcripts with SFPQ binding sites in the nucleus and prevent their translation in the cytoplasm. Therapies that reduce editing within these essential transcripts may simultaneously restore protein expression and dissolve pathological aggregation, in turn restoring neuronal function.

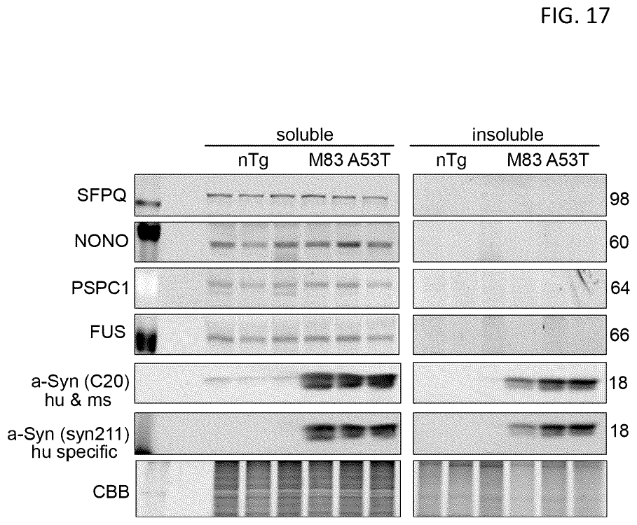

Although increased editing can potentiate RNA/protein aggregation and nuclear retention under pathological conditions, physiological RNA editing diversifies the genome by altering protein coding regions, changing mRNA splicing patterns, and regulates gene expression through the editing of Alu repeats in 3′UTRs¹⁰⁹. Further, RNA editing has been proposed to play a role in the complex development and evolution of the human brain⁹². Our studies identify a novel pathogenic feature of edited non-coding RNAs, and provide a mechanistic foundation to further examine the pathogenicity of RNAs that are extensively edited beyond the normal, physiological levels. Perturbations in this pathway and hyper-editing of human-specific Alu regions of RNA may also explain why certain pathological features of PD and DLB are unique to humans and do not occur in rodent synucleinopathy models¹¹⁰. Our attempts to validate findings in immortalized cell lines and transgenic mice expressing human A53T α-syn showed no changes in NONO/SFPQ solubility (FIG. 17) and RNA editing (not shown). This may be due to the rapid cell division of cell lines, or human-specific features of the brain including the expression of IRAlus and NEAT1_2 expression that is required for paraspeckle formation. The absence of these RNAs in rodent model systems may prevent certain features of neurodegeneration including the aggregation of RNA binding proteins that associate with NEAT1_2 such as NONO and SFPQ¹¹¹. Aggregation of RNA binding proteins occurs in multiple neurodegenerative conditions including ALS and FTD¹¹². It will be of interest in future work to assess how A-to-I editing influences the binding and aggregation of proteins involved in these diseases including TDP-43, FUS, and HNRNPA1. Our study was hypothesis-driven and focused specifically on how NONO/SFPQ aggregates influence ADAR3 expression and A-to-I editing. We focused on this pathway given that little is known regarding the effects of RNA editing in neurodegeneration. Previous work has elegantly described a mechanistic link between paraspeckles and gene regulation²⁷, as well as between NONO/SFPQ and the A-to-I editing pathway^{29,42}, providing a solid foundation to connect our results with RNA editing. However, both NONO and SFPQ play multiple roles in gene expression that were not addressed here, including RNA splicing, DNA damage repair, and miRNA targeting 33. Therefore, it is possible that these other gene regulatory functions are perturbed in PD, and future studies that address these pathways will be of interest. Furthermore, our data indicated that increased editing mainly occurred in non-coding regions of RNA including Alu repeats, however we also identified increased editing within protein coding regions. Of particular interest are glutamatergic ion channels that function in excitatory synaptic transmission including GRIA4 and GRIA2. Editing of these transcripts has the potential to alter ion permeability or downstream signal transduction. It will be critical to determine how these post-transcriptional modifications influence PD pathogenesis in future work.

Our study utilized 8-aza to inhibit A-to-I editing, given previous work that established it as an ADAR inhibitor^{93,97,98}. However, another cell culture study showed that the editing status of 3 transcripts (BPNT1, MRPS16, and ZDHHC20) were not affected by 8-aza⁹⁹. ADARs require dsRNA to bind substrates and 8-aza was provided as a free compound in our studies and in others. However, we observed a decline in A-to-I editing using four independent assays (FIG. 7A, FIG. 15A-C). It is possible that either free 8-aza, or its incorporation into dsRNA within the cell, binds the active site of ADARs and inhibits A-to-I editing of endogenous targets through competitive binding. Indeed, previous work showed that incorporation of 8-aza into polynucleotides occurs in cells⁹⁴⁻⁹⁶. We also used 8-aza at sub-toxic levels (between 0.05 and 1 uM), while others have used it at higher levels (between 1 and 10 uM) with the purpose of inducing toxicity in cancerous cell lines. At higher concentrations, it is possible that the population of cells affected most by ADAR inhibition may have died, while the surviving cells have minimal changes in A-to-I editing. It is also likely that not all transcripts will be equally affected by ADAR1 or 2 inhibition, which could be due to differences in the turnover rate of individual mRNAs, the amount of time a transcript spends in the nucleus, the duration of 8-aza treatment, and the metabolic rate of individual cell types. If an mRNA has a long half-life, longer treatment times would be required to observe changes in editing. We also observed that 8-aza increased ADAR3 levels (FIG. 8B), which could contribute to reduced A-to-I editing in our neuronal models. Finally, we confirmed our 8-aza rescue studies with lenti-ADAR3 overexpression, which resulted in similar findings compared with 8-aza (FIG. 7D, H; FIG. 8C). Our attempts at simultaneously knocking down ADARs 1 and 2 resulted in neurotoxicity (not shown), which could be due to the anti-apoptotic functions of ADARs that are independent of deaminase activity¹¹³.

Although previous studies showed that A-to-I edited mRNAs are bound and retained by NONO/SFPQ complexes^{29,38,39}, other studies concluded that physiological editing has no effect on nuclear retention, and dsRNA structure is instead responsible^{40,41}. Therefore, it is possible that some of the effects on nuclear retention we observe occur independently of A-to-I editing. However, our data indicates that destabilizing dsRNA by introducing unstable I:U base pairs promotes SFPQ binding and aggregation directly in vitro, and that only inosine-containing ssRNA co-sediments with insoluble SFPQ aggregates (FIG. 8 E-J; FIG. 16D). Therefore, our data supports the idea the pathological nuclear retention occurs from not promoting, but destabilizing dsRNA into a structure that has a higher affinity for SFPQ. However under physiological conditions, dsRNA structure may be important for nuclear retention independent of A-to-I editing. Increased protein-RNA interactions can be elevated by editing, disrupting dsRNA, and exposing protein binding sites¹⁰⁸. Our data indicates that when pathological nuclear aggregates are observed, aberrant increases in A-to-I editing beyond what is observed in physiological conditions, plays an important role in both nuclear retention and protein aggregation. This is supported by rescue studies demonstrating that reducing editing alone is sufficient to reverse pathological phenotypes in PD iPSn (FIGS. 7 and 8; FIGS. 15 and 8). Presumably, if structural characteristics of IRAlu sequences alone are responsible for the pathogenic phenotypes observed here, we would not have observed a rescue by 8-aza or ADAR3 overexpression, given that the same IRAlu sequences are present and expressed in both isogenic corrected and PD iPSn.

### Materials and Methods

iPSc Culture and Differentiation

iPSCs (induced pluripotent stem cells) were cultured on matrigel coated plates and maintained in mTESR1 media. The A53T α-syn mutation harboring iPSc and its isogenic line were kindly provided by Dr. Rudolf Jaenisch (Whitehead Institute of MIT), and were previously described and extensively characterized⁶³. iPSC harboring α-syn triplication was used in validation studies and has also been previously characterized¹⁹. For differentiation into midbrain dopaminergic neurons, iPSc lines were accutased (Corning, #25058CI) and seeded onto Matrigel (Corning, #354277) coated plates. The rest of the differentiation protocol was performed as previously described⁶³. The neurons were cultured in neurobasal medium (ThermoFisher, #21103049) with NeuroCult™ SM1 Neuronal Supplement (Stem cell technologies, #5711) and 1% glutamine and penicillin/streptomycin.

### SILAC Proteomics

The iPSC-derived neurons were labeled with light or heavy isotope containing media from day 40 for 3-7 weeks till the time of harvest (day 60-90). The neurons were cultured in regular neurobasal medium (light media) or custom formulation of neurobasal medium lacking L-lysine and L-arginine along with SM1-supplement. The custom neurobasal medium (heavy media) was supplemented with heavy-isotopes containing amino acids L-lysine (146 mg/L) and L-arginine (84 mg/L) (Cambridge isotope laboratories, #CNLM-291-H-0.5, #CNLM-539-H-0.5). The neurons were harvested and sequentially extracted into soluble and insoluble fractions as described below at day 60 and day 90. The light and heavy labeled samples from soluble and insoluble fractions were mixed at equal total protein amounts and run on an SDS-PAGE gel. The gel was stained with SimplyBlue™ SafeStain (ThermoFisher, #LC6065) and cut-out for mass-spectrometry analysis. The differential protein expression between heavy and light labeled samples were analyzed and expressed as log fold change of heavy over light for both soluble and insoluble fractions.

For Leupeptin and L-DOPA experiment, established healthy neuronal cultures were treated every other day for 30 days. The cultures were treated with vehicle (water and 0.2N Hcl in PBS) containing light isotope media or 100 uM leupeptin and 50 uM L-dopa heavy isotope containing media respectively. Sequential extraction was performed as described below into soluble and insoluble fractions for mass-spectrometric analysis.

### Sequential Extraction

IPSc-derived neuronal cultures were rinsed and harvested in cold PBS on ice. The cells were pelleted at 200×g for 5 min at 4° C. The supernatant was discarded and the pellet was used for sequential extraction of proteins. The protocol for sequential extraction was performed as previously described¹¹⁴. Briefly, the cell pellet was homogenized in 1% triton buffer supplemented with protease inhibitor cocktail (PIC) (Roche diagnostics, #11-836-170-001), 1 mM PMSF, 50 mM NaF, 2 mM sodium orthovanadate. The lysate was pelleted in an ultracentrifuge at 100,000×g, 4° C. for 30 min, re-extracted in triton to wash the pellet, followed by ultracentrifugation as before. The resulting supernatant is triton-soluble (soluble) fraction. The triton-insoluble pellet is further extracted in 2% SDS lysis buffer with PIC by boiling for 10 min, sonicating and pelleting the lysate at 100,000×g, 22° C. for 30 min. The resulting supernatant is the triton-insoluble (insoluble) fraction. The protein concentration was determined by using micro-BCA kit (ThermoFisher, #23235) and lysates were subjected to western blot analysis as described below.

For human brain tissue, a series of sequential extraction with intermediate washes was performed to obtain soluble and insoluble fractions as previously described¹⁹. Briefly, 500 mg of tissue were homogenized in a motor-driven teflon pestle and glass vessel using high-salt buffer (HSB) (50 mM Tris-HCl pH 7.4, 750 mM NaCl, 10 mM NaF, 5 mM EDTA) with protease inhibitor cocktail, incubated for 20 min on ice and centrifuged at 100,000×g for 30 minutes at 4° C. to obtain the soluble fraction. The pellets were then re-extracted with series of triton/sarkosyl buffers. The final sarkosyl-insoluble pellets were washed once with PBS and resuspended in PBS by sonication resulting in the insoluble fraction. The soluble and insoluble fractions were run on SDS-PAGE gel and analyzed by western blot.

### Western Blotting

40 ug of lysate was loaded on to tris-glycine SDS-PAGE gel (10-15% based on protein of interest). To analyze proteins with high molecular weight (>120 kDA), a tris-glycine gradient gel (ThermoFisher, #XPO4125BOX) was used. The lysates were run at 150V for approx. 1.5 hrs and then transferred onto a PVDF membrane (EMD Millipore, #IPFL00010) at 30V for 1 hr. The membrane was then post-fixed in 0.4% paraformaldehyde, washed in milliQ water and then blocked in 1:1 TBS:odyssey blocking buffer (Licor #P/N 927-40003) for 1 hr at room temp. The membrane was incubated with primary antibodies diluted in 1:1 ratio 0.2% TBS-tween and odyssey blocking buffer overnight at 4° C. The following day the membrane was washed with 0.2% TBS-Tween and incubated with secondary antibodies for 1 hr. The blot was washed as before and scanned on an odyssey imaging system. The western blots were analyzed using Image Studio software (licor) to quantify band intensities.

### Immunocytochemistry

For SFPQ/NONO staining, iPSc derived neuronal cultures were grown on poly-D-lysine/laminin coated glass coverslips. Cells were quickly rinsed with cold PBS to remove residual culture media and fixed in 4% paraformaldehyde in PBS for 15 min at room temperature (RT). The fixed cells were washed 3 times with PBS and permeabilized/blocked in 0.1% Triton X-100 with 5% normal goat serum (Jackson Immunoresearch, #005000121) and 2% BSA for 1 hr at RT. Primary antibodies NONO rabbit polyclonal dilution 1:200 (Bethyl labs, #A300-587A), SFPQ mouse monoclonal antibody (sigma, #P2860) dilution 1:200 and Tyrosine hydroxylase chicken polyclonal (EMD Millipore, AB9702) dilution 1:500 were added in 0.1% PBS-tween with 5% NGS and 2% BSA and incubated overnight at 4° C. The following day, coverslips were washed 3 times in 0.1% TBS-tween and goat Alexa fluor secondary antibodies (ThermoFisher) dilution 1:750 were added and incubated for 1 hr at RT, washed as before and mounted on glass slides with DAPI Fluoromount-G (southern biotech, #10020) for confocal microscopy. The images were acquired on a Leica confocal microscope (CTR4000/DMI4000B) with 0.3 um Z-step size covering the focal plane of the nuclei.

For NONO/Inosine staining, neurons were fixed in 4% paraformaldehyde in PBS for 15 min at RT. For the Rnase treated condition, cells after fixation were treated with Rnase (1 mg/ml, Sigma cat #70856) for 30 min, RT and rinsed with 1×PBS 3 times. Cells were permeabilized with 0.5% Triton with 200 mM RVC(NEB) for 10 min RT, washed 3 times with 1×PBS+2 mM RVC, followed by blocking with 1×PBS+2% BSA+2mMRVC for 1 hr, RT. Primary antibodies for NONO (Santacruz, 1:200) and Inosine (MBL, 1:200) in blocking solution were added and incubated at 4 C overnight, washed 3 times with 0.1% tween in 1×PBS 3 times. Secondary antibodies (Invitrogen, 1:750) were added and incubated for 1 hr at RT, followed by 3 washes with 1×PBS+0.1% tween. The cells were mounted with Prolong diamond antifade mountant (Thermofisher) and imaged.

Proximity Ligation Assay in iPSC Midbrain Neurons

Duolink® In Situ Red Starter Kit Mouse/Rabbit (Sigma, cat #DU092101) was used according to manufacturer's protocol with minor changes. iPS-derived neuronal cultures on coverslips were fixed at day 90, permeabilized as described above. The cells were blocked in Duolink blocking solution and incubated with primary antibodies rabbit anti-SFPQ (Bethyl labs, 1:200)/anti-NONO(Bethyl labs, 1:200) with mouse anti-synuclein syn211 (Sigma, 1:100) in antibody diluent overnight at 4 C. For negative control, one of the primary antibody was omitted. The next day 1× Wash buffer A and B were equilibrated to RT for all subsequent wash steps. Cells were washed with Wash A and the anti-mouse minus and anti-rabbit plus PLA probes were added and incubated for 1 hour at 37° C. Ligation was carried out for 45 min and amplification reaction for 2 hrs with wash steps and dilutions according to manufacturer's protocol. After the final wash, the cells were mounted using Duolink DAPI mountant and imaged using confocal microscope.

### Immunohistochemistry

Paraffin-embedded tissue sections on slides from human frontal cortex of healthy control and dementia with lewy bodies (DLB) patients were de-paraffinized using Histoclear II (101412-882, National Diagnostics), 3× times immersed in coplin jars for 10 min each. The slides were then immersed in descending ethanol concentration: 100% ethanol. 3× times 5 min; 95% ethanol 2× times 3 min; 70% ethanol 1×2 min and allowed to sit in deionized water. For SFPQ/NONO staining, the sections were treated with 88% formic acid for 1 min and washed with water for 5 min. Antigen retrieval was performed by placing the slides in decloaking solution (Biocare medical, #CB910M) and placed in pressure cooker for 20 min, 22 psi. The sections were removed and washed in running tap water for 5 min and allowed to sit in deionized water for 2 min. The tissue sections were immersed in blocking solution of 0.3% tritonX-100 in PBS with 3% BSA for 30 min, RT. Primary antibodies NONO 488-conjugated mouse antibody dilution 1:100 (Santacruz biotechnology, #sc-166702 AF488), SFPQ rabbit polyclonal antibody dilution 1:200 (Bethyl labs, #A301320A), were added in blocking solution and incubated overnight at 4° C. The next day sections were washed 3 times in TBS-tween for 5 min each and secondary goat Alexa fluor antibodies (ThermoFisher) were added in blocking buffer dilution 1:100 for 30 min at RT. The sections were washed as before and mounted onto slides with DAPI Fluoromount-G for image acquisition on a Leica confocal microscope.

For NONO nuclear spot count analysis, Z-Stacked images of Z-step size 1 um were captured using Leica confocal microscope (CTR4000/DMI4000B). The maximum intensity Z projection of 6 slices were used for analysis. In FIG. 1E, the spots were counted by Imaris Software by quantifying the number of puncta per nuclei, then calculating an average per individual brain sample. A total of 4 controls and 4 DLB brains were analyzed. For FIG. 11E, the images were analyzed using MATLAB2021b through the Airlocalize software¹¹⁵ as a separate validation method. Briefly, the max intensity projection images for each condition were opened through Airlocalize and the spots to be detected were sampled randomly for a local Gaussian fit. The detection threshold value=45 was set for positive spot signal. The spots outside the nucleus were excluded and average integrated intensity was used for analysis.

### Human Brain Nuclear Inclusion Analysis

Images acquired by confocal microscopy with 1 um Z-step size were imported into Imaris 9.9 and analyzed using the cell module with the DAPI channel as cell and NONO/SFPQ inclusions as two vesicle types with respective channels. The Cell Background Subtraction Width was set to 5-7.00 μm and estimated vesicle diameter to 0.7 μm. Non-nuclear objects were manually discarded. The Vesicle Background Subtraction and enable region growing for each vesicle type was selected and the filter quality above automatic threshold was applied. The intensity mean was manually thresholded for each channel and respective vesicle type. Vesicles outside nucleus were manually discarded and only vesicles within the nucleus were analyzed. The creation parameters were saved for batch process to apply for different control and DLB groups. The statistic attributes were exported through vantage plot and calculated vesicle diameter for each vesicle type per image or field of view was used for plotting the graph.

### Assessment of Co-Aggregation of SFPQ and α-Synuclein in Cell Free Systems.

A 40 kDa truncated form of recombinant SFPQ (residues 276-598) was purified as described previously 43, stored in 25% glycerol/PBS pH7.4. This region of SFPQ contains domains RRM1, RRM2, NOPS, and coiled-coil domain. Just before use, glycerol was removed from SFPQ monomers by buffer exchange into PBS pH 7.4 (3× each using 3 volumes of PBS in 10,000 MWCO filters (Millipore)). Samples were spun at 100,000×g for 1 hr to remove any pelletable material, then used for experiments, α-syn full length monomers were isolated as described 21. Oligomers were generated by lypholization as previously described in detail^{81,82}. Pre-formed fibrils (PFFs) were generated according to standardized protocols¹¹⁶, by shaking/incubating monomers for 7 days, 1000 RPM, 37° C., centrifuged at 100,000×g for 1 hour, then sonicated to generated fragmented fibrils. A-syn forms were incubated with monomeric SFPQ with both proteins at 1 mg/ml, and incubated/shaken at 1000 RPM, 37° C. for the indicated lengths of time. Samples were separated into supernatant and pelletable fractions by centrifugation at 100,000×g, 4° C., 1 hr, and lug protein was analyzed by western blot (using SFPQ D8 antibody (mouse) and α-synMJFR-1 antibody (rabbit) (Abcam)). The different conditions (SFPQ+BSA, +Mon, +Olig, or +PFF) at 24 and 48 hr time points were loaded together on the same blots and detected using fluorescent conjugated secondary antibodies (Alex-647-anti-mouse; Alexa 790-anti-rabbit) for accurate quantification. For kinetic analysis, the individual time points from the same experimental condition were loaded together on the same blot, to quantify the change over time. Colocalization of SFPQ and α-syn was done on the same blot using two different colored secondary antibodies. Samples were analyzed by Thioflavin T (ThT). 10 mM ThT stock was made fresh prior to each experiment by dissolving in water, then diluting to 10 uM in pH 8.5 glycine buffer. 10 uM ThT was added to 5 μg of total protein, incubated for 5 minutes in 96 well black fluoroplates (Nunc), and analyzed in a Molecular Devices SpectaMax M5 fluorescent plate reader (ex=450, em=485, cutoff=470 nm).

### Computational Analysis of RNA Secondary Structure

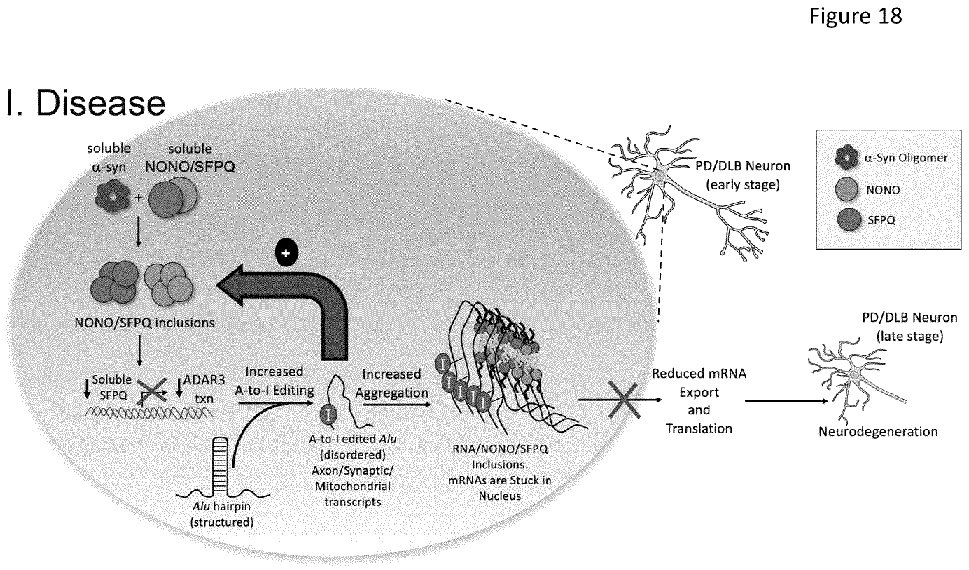

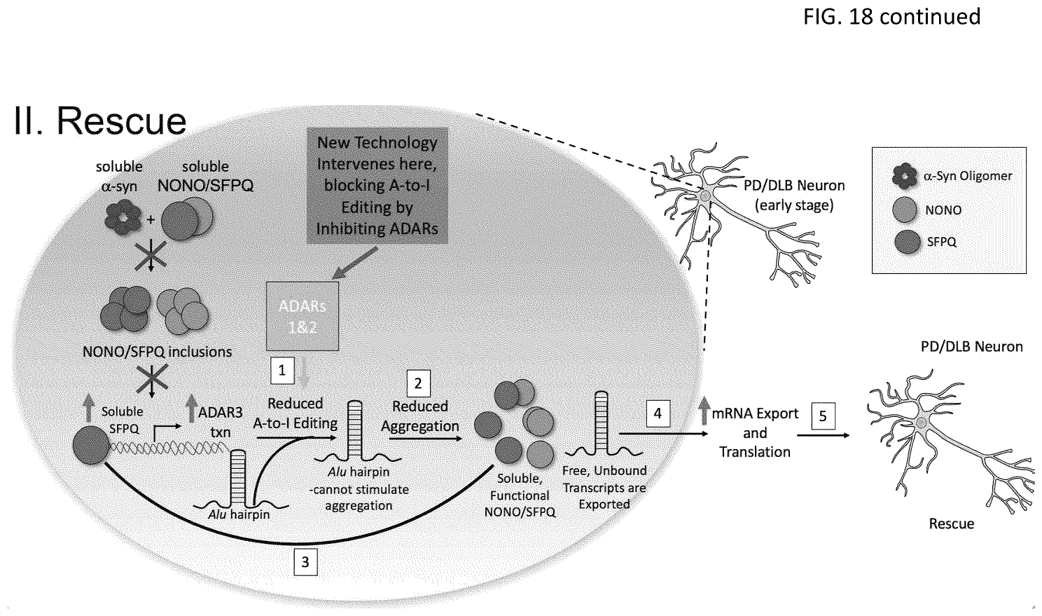

A 1045 bp region of the 3′UTR of PRKAR2A obtained from NM_00457.4 (starting position 2562; corresponding to Chr3+: 48749472-48750516) was analyzed by RNA fold (rna.tbi.univie.ac.at/cgi-bin/RNAWebSuite/RNAfold.cgi)¹¹⁷. Results from the MEF and Centroid secondary structures were used to determine structural changes in the 5 hyper-edited sites identified from RNA-seq. Each adenosine identified to be hyper-edited in PD iPSn by RNA-seq was changed to inosine either individually or in combination, and used as the input for RNAfold. Only constructs where site 2 (position 2656 of NM_00457.4) was converted to inosine resulted in any predicted changes in structure. The Centroid structures are shown in FIG. 18 A. The same procedure was used to analyze the 31 bp oligonucleotide region, which starts at position 2650 in NM_00457.4. Structural alterations were confirmed by gel electrophoresis and RNAse protection assays.

### Functional Analysis of the 3′UTR of PRKAR2A

A 1045 bp region of the 3′UTR of PRKAR2A obtained from NM_00457.4 (starting position 2562; corresponding to Chr3+: 48749472-48750516) was subcloned downstream into eGFP reporter plasmid. The cloning of EGFP with or without PRKAR2A 3′ UTR was custom ordered through VectorBuilder under Synapsin I or EF1A promoter into pLV lentiviral backbone. The 3′ UTR of PRKAR2A gene consisting of 1045 bp fragment (Chr3+:48749472-48750516) was cloned after EGFP gene with stop codon. The lentivirus was prepared, and neurons were transduced as described above. iPSn cultures were prepared for western blot analysis of GFP as described above. For HEK experiments, the plasmids were transfected using lipofectamine 2000 and fixed for FISH analysis 24 hours later.

### Gel Electrophoresis Analysis of RNA

RNA oligonucleotides (Horizon Discovery, sequences shown in FIG. 18B) were re-constituted in PBS at 100 uM. Oligonucleotides were prepared by diluting the samples to 500 nM in water and 1×RNA loading dye (New England Biolabs (NEB), #B0363S), heating to 80° C. for 15 minutes, followed by cooling at 25° C. for 15-30 minutes. Native gel electrophoresis was done by loading 10 ul of 500 nM oligos in TBE-PAGE gels (15% acrylamide). It was essential that the gels were pre-run at 200 mV for 1 hr followed by washing out of the wells prior to loading the samples. Molecular weight was estimated by the migration of bromophenol blue (15 nt for a 15% TBE gel), the low ssRNA marker (NEB #N0364S) and the microRNA marker (NEB #N2102S). For denaturing electrophoresis, samples were prepared in the same way but loaded on TBE-gels containing 8M Urea. Gels were pre-run at 200V for 45 minutes-to-1 hr (this step is essential), wells were washed out, and 10 ul of each sample was loaded and run for an additional 45 minutes to 1 hour. Gels were developed in 1:5000 dilution of SYBR Green II RNA stain (ThermoFisher #S7586) in 1×TBE for 20-to-45 minutes, then scanned on a Biorad gel Doc imager. Images were analyzed by Image J software to obtain peak migration in cm and integrated pixel intensity of the bands. The molecular weight (MW) in nucleotides (nt) was estimated by comparisons with the RNA ladders mentioned above and bromophenol blue). The assay was repeated 8 times, which represents 4 separate reaction tubes set up on two different days.

### RNA Stability Assays by RNAse Digestion

Oligo stocks of the same sequence used in FIG. 18B were diluted to 1 uM in RNA Structure Buffer (10 mM Tris-Cl, pH 7.4, 100 mM KCl, 10 mm MgCl2), heated to 80° C. for 15 minutes, and cooled to 25° C. for 15-to-30 minutes. RNAse T1 (ThermoFisher #EN0541, 1000 U/ul) was added at a 1:1000 dilution in a 10 ul final volume reaction and incubated at 25° C. for 15 minutes. The sample was mixed with 10 ul of 2×RNA loading dye (NEB #B0363S) for a final volume of 20 ul, and heat-killed at 100° C. for 10 minutes. In parallel, 1 uM oligos were digested with RNAse I (ThermoFisher #EN0601, 10 U/ul) at 1:100 and 1:1000 dilutions and incubated at 37° C. for 20 minutes, followed by mixing with 2×NEB loading dye and heat-kill for 10 minutes at 100° C. All 20 ul of sample was loaded on TBE-UREA gels (15% acrylamide) as described above, pre-running the gels before sample loading. Gels were developed in 1:5000 dilution of SYBR Green II RNA stain (ThermoFisher #S7586) in 1×TBE for 20-to-45 minutes, then scanned on a Biorad gel Doc imager. The RNAse digestion assays were set up in 3 different reaction tubes on 2 separate days to assess reproducibility. The gels were not quantified because the inosine-containing oligos had no detectable signal after digestion.

### RNA-SFPQ Binding In Vitro

6-FAM-labeled oligo stocks of the same sequence used in FIG. 18B (Horizon Discovery) were diluted to 100 nM in RNA Structure buffer (10 mM Tris-Cl, pH 7.4, 100 mM KCl, 10 mm MgCl2), heated to 80° C. for 15 minutes and cooled to 25° C. for 15-to-30 minutes. 100 nM RNA oligos were mixed with recombinant purified SFPQ at 1, 2, 5, and 10 uM for 20 minutes at 25° C. in a 10 ul reaction, mixed with Novex™ Hi-Density TBE Sample Buffer (5×) (ThermoFisher #LC6678), and analyzed on TBE-polyacrylamide gels (8%). It was essential that the gels were pre-run at 200 mV for 1 hr followed by washing out of the wells prior to loading the samples. After the samples were electrophoresed, gels were directly scanned on an Azure Sapphire imager using the 532 nm optical module. Binding reactions were set up in 3 different reaction tubes on different days to assess reproducibility. Gels were quantified using integrated intensity signals obtained from Licor Image studio software.

### RNA-SFPQ Aggregation Assays

Native Gel Electrophoresis: RNA oligos were diluted to 2 nM in PBS and mixed with 250 nM SFPQ in a 10 ul reaction volume, and incubated at 25° C. for 20 minutes. Samples were mixed with Novex™ Hi-Density TBE Sample Buffer (5×) (ThermoFisher #LC6678) and the entire sample was loaded on native TBE polyacrylamide gels (6%). Gels were transferred to PVDF membranes as described under “Western Blot Analysis” and probed with anti-SFPQ antibody (D8, Santa Cruz). Gel images were scanned on Azure Sapphire Imager and quantified using Licor Image Studio software. The assay was set up three times on separate days to assess reproducibility of the aggregation. High molecular weight oligomers were defined as species that migrated slower compared to the ‘SFPQ alone’ species.

Sedimentation Analysis: RNA oligos were diluted to 1 uM in PBS and mixed with 10 uM (final) recombinant SFPQ in a 10 ul reaction volume. SFPQ protein was prepared in the same way as described above, “Assessment of co-aggregation of SFPQ and α-synuclein in cellfree systems”, by buffer exchange in PBS to remove the glycerol present in the storage buffer immediately before setting up the assay. Samples were incubated for 10 minutes at 25° C. then centrifuged at 100,000×G, 4° C. for 1 hour. Supernatant and pellet fractions were separated and analyzed by either western blot analysis for SFPQ aggregation as described above, or RNA electrophoresis by TBE-Urea gels (15% acrylamide)/SYBR green. Quantifications of both were done using Licor Image Studio and analyzed by measuring the percent of SFPQ or RNA present in the pellet fraction. The assay was repeated 4-to-5 times in separate reaction tubes, on 2 different days to assess reproducibility.

### Dual-Luciferase Reporter Assay

iPSc derived dopaminergic neurons were plated on poly-D-lysine/laminin coated 96-well plate at seeding density of 80,000-100,000 cells per well. Briefly, in a centrifuge tube 25 ul of Opti-MEM reduced serum media (Thermo Fisher, #31985-070) was mixed with 1.2 ul of lipofectamine 2000 (ThermoFisher, #11668019) at RT for 5 min. In a second tube, 25 ul of Opti-MEM along with 500 ng of pLightswitch-ADAR3 promoter-renilla luciferase plasmid (Active motif, #S703748) and 100 ng of control constitutive promoter driven pGL2-SV40-firefly luciferase plasmid (Addgene, ##26280) were mixed at RT,5 min. The reactions containing lipofectamine 2000 and plasmids were mixed, incubated at RT for 20 min and co-transfected onto neurons (per one well) in neurobasal medium with sm-1 supplement. The neurons were lysed 48 hrs post-transfection using 75 ul lysis buffer per well from Dual-Glo® Luciferase Assay System (Promega, #E2920) for 30 min at RT. The luminescence of renilla and firefly luciferase was measured according to manufacturer protocol using a spectramax plate reader. The ADAR3 promoter activity was expressed as the luminescence unit of Renilla luciferase normalized to firefly luciferase.

### RNA-Fluorescent In Situ Hybridization (FISH)

For iPSc-neurons, the cells on coverslips were fixed in 4% PFA in PBS for 20 min at RT. The PFA was quenched for 10 min by adding 125 mM Glycine with 10 mM Ribonucleoside Vanadyl Complex (RVC) (New England Biolabs, #S1402S). The cells were washed 3 times in PBS with 2 mM RVC and permeabilized with 0.1% triton in RNAse free water with 2 mM RVC for 15 min at RT. Stellaris RNA-FISH reagents (LGC biosearch technologies) were used to perform the polyA+RNA-FISH. Briefly, the permeabilized cells were then incubated with wash buffer A with 10% formamide for 30 min at RT. For hybridization and detection of polyA+RNA, a custom Stellaris biotin conjugated oligo(dt)₃₀ probe was used at 125 nM final concentration, 37° C., overnight in a humidified chamber. Next day, the cells were washed with washed buffer A with 10% formamide twice for 30 min each, followed by wash buffer B for 5 min. The cells were then blocked in 2% BSA in PBS with 2 mM RVC for 30 min RT and streptavidin secondary antibody in block solution (ThermoFisher) was added for 1 hr at RT. The cells were washed with 0.1% PBS-tween 3 times and mounted with DAPI Fluoromount-G for image acquisition on a confocal microscope (Z-step of 0.5 um). Images were than imported to ImageJ for analysis and converted into binary image and intermodes thresholded to count for polyA+ nuclei. For analysis of mRNA inclusions of CADPS and CYFIP2, images from 3-to-4 separate culture wells were analyzed for the number of cells per field of view that contained total puncta as well as puncta of >1 um in diameter and normalized to DAPI signal. Data were plotted as both individual cells containing nuclear mRNA puncta (of all sizes) and an average of the cells that only contained the large puncta (defined as >1 um in diameter).

For RNA-FISH with IHC in human frontal cortex tissue paraffin sections, the tissue was deparaffinized and subjected to antigen retrieval as described above (formic acid treatment was excluded). The tissue sections were then permeabilized in 0.3% TritonX-100 in PBS with 10 mM RVC for 15 min RT, washed 5 min in 1×PBS+RVC. The tissue sections were then blocked for biotin using endogenous biotin blocking kit (ThermoFisher, #E21390) and subjected to stellaris RNA-FISH protocol as mentioned above using biotin conjugated-oligo(dt)₂₅ probe for overnight hybridization. The next day, the sections were washed with wash buffer A and B as before, followed by incubation of primary antibody anti-NONO rabbit polyclonal dilution 1:200 (bethyl labs), anti-NeuN mouse monoclonal dilution 1:1000 (EMD Millipore, #ABN91) and streptavidin secondary for oligo-dt RNA probe in PBS with 0.3% with 3% BSA at 4° C. overnight. The next morning, tissue sections washed 3 times with 0.1% PBS-tween for 5 min and goat secondary Alexa fluor antibodies (ThermoFisher) were added for 30 min at RT. The sections were washed as before and mounted with DAPI Fluoromount-G for imaging using a Leica confocal microscope. The colocalization analysis for NONO and polyA+RNA was done using coloc2 plugin in ImageJ software on NeuN positive nuclei using the triangle threshold.

### Cytoplasmic-Nuclear RNA Fractionation

iPSc-neurons at seeding density of 1-2 million per well were used as starting material for fractionation purposes. The cytoplasmic-nuclear fractionation was performed using the NE-PER kit (ThermoFisher, 78833). Briefly, the neurons were extracted using CER-I buffer with betamercaptoethanol (BME) and centrifuged to get the nuclear pellet. The nuclear pellet was washed in CER-I to prevent carry over into nuclear fraction. Once the cytoplasmic supernatant and nuclear pellet were obtained, the RNA was extracted using Sureprep nuclear or cytoplasmic RNA purification kit (Fisher scientific, #BP280550). Parallel wells for each experimental condition or genotype (n=3-4) were harvested for total RNA extraction. The RNA concentrations were measured on nanodrop 2000 (ThermoFisher). The cDNA synthesis was performed using 500-1000 ng of total RNA from each cellular compartment and total fraction as described below.

### RNA Extraction and Quantitative-RT-PCR

Total RNA from iPSC—neurons was extracted using the PureLink RNA mini kit (ThermoFisher). 1000 ng of total RNA was treated with DNAseI (Thermofisher) to remove residual genomic DNA at 37° C. for 30 min. Then, cDNA synthesis was performed using RevertAid First Strand cDNA Synthesis Kit (Thermofisher).Quantitative-real-time PCR (Q-RT-PCR) was performed using predesigned TaqMan probes. The quantification was based on delta-ct method normalized to beta-actin levels as fold change. Each target mRNA was measured with n=2 technical replicates per biological replicate.

For Nuclear-cytoplasmic ratio, RNA was fractionated as described above and parallel wells were harvested for total RNA extraction for each experimental condition. Delta Ct method was used to calculate the RNA levels of cytoplasmic or nuclear fractions relative to total (cytoplasmic+nuclear) target transcript levels and represented as fold change.

For RNA extraction from human brain frontal cortex, 40-50 mg of brain tissue per sample was used and Total RNA was extracted with TRIzol™ Reagent (ThermoFisher, #15596026). The aqueous phase was used and mixed with equal volumes of 70% ethanol. The rest of the extraction was performed using the PureLink RNA mini kit (ThermoFisher) and cDNA synthesis was performed as mentioned above.

### RNA Dot-Blot

Total RNA or Cyto/nuclear RNA was isolated from neurons as described above. For polyA+ RNA isolation, total RNA or fractionated RNA was used and subjected to purification using μMACS™ mRNA Isolation Kit (Miltenyi Biotec, cat #130-075-201) according to manufacturer's protocol. 30 ng of RNA in RNAse free water was heated to 95 C in a PCR tube for 3 min and chilled briefly on ice. 2 ul of sample was blotted onto Biodyne™ B Nylon Membrane (Thermofisher, cat #77016), followed by UV-crosslinking at 125 mJ/cm2 for 20-40 s. Wash the membrane in 10 mls of 0.1% TBS-tween (TBST) for 5 min RT and block in 0.1% TBT+2% BSA for 1 hr RT. Anti-Inosine antibody (MBL life science, cat #PM098, 1:1000) was added in blocking solution and incubated at 4 C overnight. The membrane was washed 3 times with 0.1% TBST and incubated with HRP-conjugated secondary (1:5000) 1 hr RT, followed by 3 washes with 0.1% TBST and developed using Clarity Max™ Western ECL Substrate (Biorad, cat #1705062S). The membrane was imaged in Biorad Chemidoc imaging system.

Assessment of Inosine Content in polyA mRNA by HPLC-MS Analysis.

Total polyA mRNA was extracted using μMACS™ mRNA Isolation Kit (Miltenyi Biotec, cat #130-075-201). 500 ng of polyA RNA was digested using Nucleoside Digestion Mix (NEB, cat #M0649S). Samples were submitted to the metabolomics core at the Metabolite Profiling Core Facility at the Whitehead Institute of the Massachusetts Institute of Technology (biology.mit.edu/tile/metabolite-profiling-core-facility). The samples were analyzed by HPLC-MS and elution profiles were compared to purified standards for each nucleotide. Raw peak areas were used to quantify the relative amount of each nucleotide and expressed as fold change compared to the isogenic control line. The assay was repeated in 4 individual culture well replicates. For quality control (QC) analysis, a “pool” of sample consisting of a mixture of several uL from each of the biological samples was run, which created a representative sample that was run multiple times to get a measure of technical reproducibility for each nucleotide. A CV (standard deviation/average) was calculated for these technical replicates and metabolites with a CV<0.25 were considered reliably detected. Also, a larger injection volume was run with 0.3-fold and 0.1-fold dilutions of the pooled sample, which indicated whether samples fall in the linear range of detection for each metabolite, or there was a detector saturation or nearing the lower limit of detection.

gDNA Extraction

Genomic DNA from iPSC-neurons was extracted using PureLink Genomic DNA extraction kit (ThermoFisher). For the human brain tissue, the organic phase from the trizol extraction above was utilized and 400 ul of ethanol was added. The samples were centrifuged at max speed to pellet gDNA. The supernatant was discarded and rest of the extraction was performed using the PureLink Genomic DNA kit. The concentration and purity of genomic DNA was measured on nanodrop 2000 (ThermoFisher).

### A-to-I Editing and Sanger Sequencing

For detection of A-to-I site in iPSc-neurons or human brain tissue, 100 ng of cDNA or gDNA was used as template for amplification of target gene using touchdown PCR. The PCR amplification was performed using Q5 polymerase (NEB) in 20 ul reaction with following protocol: 98° C. 30 s; 12 cycles: 98° C. 10 s, 65° C.-55° C. 30 s (−1° C./cycle), 72° C. 15 s; 18 cycles: 98° C. 10 s, 55° C. 30 s, 72° C. 15 s; final extension 72° C. 1 min. The PCR products were then treated with ExoSAP-IT™ PCR Product Cleanup Reagent (ThermoFisher, #78200.200.UL). The PCR products were run on an agarose gel to confirm the size and specificity of amplified product before submitting to Sanger sequencing.

The raw Sanger sequencing chromatogram files (.abl file) were opened in Quality Check (QC) Module on thermocloud platform (ThermoFisher). The peak traces were exported as .csv files to obtain the peak heights of the base call. The peak heights for bases A and G at edit site were obtained and editing percentage was calculated as: Peak height of “G”/(Peak height of “A”+peak height of “G”)×100.

Treatment of iPSC-Neurons with ADAR Inhibitor 8-Aza-Adenonsine.

IPSC-neurons were treated from day 60 or day 90 in culture for 30 days with vehicle (DMSO) or 200 nM of ADAR inhibitor 8-aza-adenosine (8-aza). The vehicle or 8-aza was diluted in neurobasal media with sm-1 supplement and added onto cultures three times per week until the day of harvest for use in downstream assays.

To test 8-aza in HEK cells, we utilized an RFP—editing site linker-GFP reporter construct, a gift from Dr. Michael F. Jantsch (University of Vienna)¹⁰⁰. The stem-loop linker belonging to established editing substrate glutamate receptor B (GluR-B) contains the edit site encoding for stop codon (UAG). In the presence of A-I editing, the stop codon (UAG) is converted into W (UGG) leading to downstream GFP being transcribed. The ratio of GFP/RFP provides a measure of A-I editing activity in transfected cells. HEK cells were transfected with 250 ng of reporter plasmid using Xtreme gene HP DNA transfection reagent (Sigma, #6366236001). The cells were then treated with DMSO, 50 nM, 100 nM and 250 nM 8-aza for 5 days. The cells were fixed and imaged using Leica confocal microscope (CTR4000/DMI4000B).

### Lentiviral Transduction

For ADAR hyperactive mutants ADAR1 E713Q and ADAR2 E488Q the CDS were custom cloned into pLV lentiviral backbone (VectorBuilder) under synapsin promoter. Flag-tag-ADAR3 was cloned into pER4 lentivector at BmtI/PciI site under the PGK promoter. The lentiviral vectors were then packaged as previously described (Cuddy et. al 2019). The lentivirus was concentrated using LentiX concentrator (Takarabio) and titered with HiV1-p24 ELISA kit (Zeptometrix). The neurons were transduced at MOI 3 at assay timepoint and harvested 14 days post infection for western blot analysis or 30 days post infection for ADAR3 oligo-dT FISH assay.

### Synaptic Puncta Colocalization Analysis

Immunocytochemistry of day 120 iPSC-derived mid-brain neuronal cultures were performed as described above. Primary antibodies for pre-synaptic marker mouse monoclonal Synaptophysin-1 (Synaptic systems #101 011) dilution 1:250, post-synaptic marker rabbit polyclonal PSD-95 (Synaptic systems #342 403) dilution 1:250 and chicken polyclonal neuronal specific-beta III Tubulin (Abcam #ab41489) dilution 1:500 was used. Z-stacked Images with step-size of 0.5 um were acquired using the Leica confocal microscope (CTR4000/DMI4000B). The standard deviation Z-projection of 8 slices was used for image analysis using the Synapse Counter plugin in ImageJ software¹¹⁸. Images were converted into binary with default setting in ImageJ software for analysis. The colocalization analysis of pre- and post-synaptic puncta was based on previously experimentally determined synaptic puncta size4.To set the puncta size, the default parameters of minimum=10 px2; maximum=400 px2 were used for pre-synaptic and post-synaptic compartment size. The rolling ball radius=5 was set for local background subtraction of the puncta. The total number of puncta for each compartment, and colocalized pre- and post-synaptic puncta was obtained through the output window. The colocalized puncta is automatically defined as pre- and post-synaptic compartments having overlap of 33-100%. The number of synaptophysin-PSD95 colocalized positive puncta was normalized to total synaptophysin puncta number per image and represented as fold change.

### Lactate Dehydrogenase (LDH) Cytotoxicity Assay

To measure neuron viability in iPSc-neurons, we used CyQUANT™ LDH Cytotoxicity Assay to measure LDH activity according to manufacturers' protocol. Briefly, 50 ul media from vehicle or 8-azaa 30 day treated neurons was incubated with 50 ul of reaction mixture in 96-well microplate for 30 min at room temperature. The reaction was stopped and absorbance was measured at 490 nm and 680 nm in a molecular devices plate reader. The LDH activity was determined by subtracting the absorbance at 680 nm from absorbance at 490 nm and expressed as fold change.

### Gene Ontology and Enrichment Analysis of Proteomics and RNA-Seq Data

The data from proteomics was ranked according to fold change and significance (FDR q<0.05) and imported into g:Profiler (Raudvere et al., 2019) as ordered query. The significance (p<0.05) and threshold parameters were set as default setting. The terms having less than 4 genes or more than 500, redundant parent terms were discarded. The reactome pathway and gene ontology (GO) terms were exported as .gem files. The enrichment map was generated using cytoscape (v3.9.0) and Enrichment map plugin (Merico et al., 2010). The size of the node indicates the number of proteins, color represents the p-value and the name around the clusters represents the major pathway.

### Computational Analysis for Solubility Correlations

The proteins with significant solubility changes from day 90 A53T iPSn proteomics data set was used for studying physicochemical and other intrinsic parameters. The canonical FASTA protein sequences were obtained as bulk through Retrieve/ID mapping feature on uniprot database (uniprot.org/uploadlists/). The PLAAC database was used to study prion-like amino acid composition (plaac.wi.mit.edu/) using the default parameters⁶⁸. The output LLR (log-likelihood ratio) was used for analysis. For supersaturation analysis, previously published folded and unfolded supersaturation score were used and plotted against solubility¹¹⁹. Secondary structure parameters of percent coiled-coiled, alpha-helix, beta-sheet and intrinsic disorder regions were analyzed through the PASTA 2.0 database (old.protein.bio.unipd.it/pasta2/)¹²⁰. Basic intrinsic properties such as isoelectric point, charge and aliphatic index were calculated using the Protean 3D software (dnastar.com/software/lasergene/protean-3d/). Simple linear regression analysis was performed between the above parameters and decreased solubility to study correlations using GraphPad Prism software.

### Detection of A-to-I Editing Using RNA-Seq and Whole Genome Sequencing

Total RNA and genomic DNA from day 60 iPS neurons were extracted using Purelink RNA mini kit and Purelink genomic extraction kit (Thermofisher). The Quality control check, library preparation and sequencing were performed using services provided by Novogene Co, Ltd. (novogene.com/us-en/). For A-I editing RNA-seq, total RNA of all samples was analyzed using bioanalyzer and 1.5-2 ug of total RNA with RIN score 9.7-10 was used for downstream polyA+ mRNA library preparation and sequencing. Deep sequencing was performed using Human mRNA Sequencing (WOBI) service (Novogene) to obtain 300M reads PE150 per sample with Novaseq 6000 platform. The whole genome sequencing (WGS) of A53T iPSn and isogenic control was performed using Human Whole Genome Sequencing (WOBI) service (Novogene) to obtain PE150 using Novaseq 6000 platform. The subsequent analysis was performed in-house as described below.

### Whole-Genome Sequencing Variant Calling

Quality control (QC) for whole-genome sequencing (WGS) reads was performed using FASTQC v.0.11.5 (bioinformatics.babraham.ac.uk/projects/fastqc). Read alignment to the human reference genome build hg38 was done using the Burrows-Wheeler Aligner mem option (BWA v.0.7.17) 121. Germline variants were called using the Genome Analysis ToolKit best practices 5 pipeline version 4.1.8 122. Briefly, aligned reads in bam format were marked for duplicates, sorted by coordinates and recalibrated using the base quality scores (BQSR). Next, the HaplotypeCaller tool was used to call variants in gVCF format for each chromosome separately, then BCFTools v.1.10.0¹²³ concat option was used to concatenate each gVCF into a single file containing all chromosomes. GenomicsDBimport and GenotypeFromDB were used to merge sample-wise gVCFs and call raw variants, respectively. Hard filtering and Variant Quality Score Recalibration (VQSR) were used to filter bad quality variants. Post-GATK QC's were done using BCFTools v.1.10.0, removing variants without PASS in the FILTER field, with read depth (DP)<10, genotype quality (GQ)<20, indel left-normalization, and multiallelic splitting.

### RNA-Seq Variant Calling

RNAseq reads were similarly analyzed for QC using FASTQC v.0.11.5 as WGS reads. We followed GATK directions using the RNAseq short variant discovery pipeline (gatk.broadinstitute.org/hc/en-us/articles/360035531192-RNAseq-short-variant-discovery-SNPs-Indels-). Briefly, exon junction-aware alignment to the human reference genome build hg38 was performed using the STAR v.2.7.5¹²⁴ with the 2-pass mode, followed by mark duplicates, splitting reads with N cigar markers, and base quality recalibration. Sample-wise VCFs were generated using HaplotypeCaller, including non-variant sites to compare genotypes across samples. Variant filtering was done with RNAseq specific settings to obtain good quality variants. Sample-wise filtered VCFs were merged using BCFTools v.1.10.0 merge command.

### Detection of RNA A-to-I Editing Sites

VCF files from WGS and RNASeq variant calling were merged using BCFTools v.1.10.0 merge command. Custom text editing scripts were made to select the following variants from RNASeq per sample: i) The canonical editing from A to G in the forward strand; and ii) T to C in the reverse strand. For (i), we compared genotypes of each site to the genotypes obtained in the WGS variant call set, and we kept variants if there were homozygous references (0/0 for A/A or T/T). We also selected sites if the WGS genotypes were homozygous alternatives (1/1 for A/A or T/T), and the RNAseq genotypes were either heterozygous or homozygous containing the edited bases G or C. The set of candidate editing sites were annotated with ANNOVAR¹²⁵, including variant localization (exonic, intronic, intergenic, etc.) and distance to nearby genes. The A-I RNA editing database REDI¹²⁶ was used to annotate the candidate editing sites with known A-I changes, including overlap with Alu and repeat regions. Read abundance ratio for editing sites was performed on each sample taking the alternative allele depth divided by the site allele depth. Two-tailed t-tests were done using the t.test function in R, to statistically compare the read abundance ratios between the mutated (M) and corrected (C) samples. Multiple testing correction was done using a false-discovery rate of <0.05, using the Benjamini-Hochberg method.

### A-to-I RNA Editing Heatmap Analysis

Differentially expressed A-I edit sites from RNAseq of day 60 neurons were imported into Morpheus analysis tool (software.broadinstitute.org/morpheus/) in a form of excel spreadsheet matrix with editing values for each genotype as data matrix, gene names as row annotations and location/transcript type of edit sites as column annotations. The dendrogram was generated using hierarchical clustering of column annotations. The heatmap was saved and exported as .png or .pdf file.

### REFERENCES

1. Lansbury, P. T., and Lashuel, H. A. (2006). A century-old debate on protein aggregation and neurodegeneration enters the clinic. Nature 443, 774-779. 10.1038/nature05290.
2. Spillantini, M. G., Schmidt, M. L., Lee, V. M., Trojanowski, J. Q., Jakes, R., and Goedert, M. (1997). Alpha-synuclein in Lewy bodies. Nature 388, 839-840. 10.1038/42166.
3. Braak, H., Del Tredici, K., Rub, U., de Vos, R. A., Jansen Steur, E. N., and Braak, E. (2003). Staging of brain pathology related to sporadic Parkinson's disease. Neurobiol Aging 24, 197-211. 10.1016/s0197-4580(02)00065-9.
4. Kuzuhara, S., Mori, H., Izumiyama, N., Yoshimura, M., and Ihara, Y. (1988). Lewy bodies are ubiquitinated. A light and electron microscopic immunocytochemical study. Acta Neuropathol 75, 345-353. 10.1007/BF00687787.
5. Goldman, J. E., Yen, S. H., Chiu, F. C., and Peress, N. S. (1983). Lewy bodies of Parkinson's disease contain neurofilament antigens. Science 221, 1082-1084. 10.1126/science.6308771.
6. Polymeropoulos, M. H., Lavedan, C., Leroy, E., Ide, S. E., Dehejia, A., Dutra, A., Pike, B., Root, H., Rubenstein, J., Boyer, R., et al. (1997). Mutation in the alpha-synuclein gene identified in families with Parkinson's disease. Science 276, 2045-2047. 10.1126/science.276.5321.2045.
7. Singleton, A. B., Farrer, M., Johnson, J., Singleton, A., Hague, S., Kachergus, J., Hulihan, M., Peuralinna, T., Dutra, A., Nussbaum, R., et al. (2003). alpha-synuclein locus triplication causes Parkinson's disease. Science 302, 841. 10.1126/science.1090278.
8. Braak, H., Sandmann-Keil, D., Gai, W., and Braak, E. (1999). Extensive axonal Lewy neurites in Parkinson's disease: a novel pathological feature revealed by alpha-synuclein immunocytochemistry. Neurosci Lett 265, 67-69. 10.1016/s0304-3940(99)00208-6.
9. Duda, J. E., Giasson, B. I., Mabon, M. E., Lee, V. M., and Trojanowski, J. Q. (2002). Novel antibodies to synuclein show abundant striatal pathology in Lewy body diseases. Ann Neurol 52, 205-210. 10.1002/ana.10279.
10. Tanji, K., Mori, F., Mimura, J., Itoh, K., Kakita, A., Takahashi, H., and Wakabayashi, K. (2010). Proteinase K-resistant alpha-synuclein is deposited in presynapses in human Lewy body disease and A53T alpha-synuclein transgenic mice. Acta Neuropathol 120, 145-154. 10.1007/s00401-010-0676-z.
11. Lin, L., Lesnick, T. G., Maraganore, D. M., and Isacson, O. (2009). Axon guidance and synaptic maintenance: preclinical markers for neurodegenerative disease and therapeutics. Trends Neurosci 32, 142-149. 10.1016/j.tins.2008.11.006.
12. Soukup, S. F., Vanhauwaert, R., and Verstreken, P. (2018). Parkinson's disease: convergence on synaptic homeostasis. EMBO J 37. 10.15252/embj.201898960.
13. Cheng, H. C., Ulane, C. M., and Burke, R. E. (2010). Clinical progression in Parkinson disease and the neurobiology of axons. Ann Neurol 67, 715-725. 10.1002/ana.21995.
14. Chia, R., Sabir, M. S., Bandres-Ciga, S., Saez-Atienzar, S., Reynolds, R. H., Gustavsson, E., Walton, R. L., Ahmed, S., Viollet, C., Ding, J., et al. (2021). Genome sequencing analysis identifies new loci associated with Lewy body dementia and provides insights into its genetic architecture. Nat Genet 53, 294-303. 10.1038/s41588-021-00785-3.
15. Chang, D., Nalls, M. A., Hallgrimsdottir, I. B., Hunkapiller, J., van der Brug, M., Cai, F., International Parkinson's Disease Genomics, C., andMe Research, T., Kerchner, G. A., Ayalon, G., et al. (2017). A meta-analysis of genome-wide association studies identifies 17 new Parkinson's disease risk loci. Nat Genet 49, 1511-1516. 10.1038/ng.3955.
16. Klein, A. D., and Mazzulli, J. R. (2018). Is Parkinson's disease a lysosomal disorder?Brain 141, 2255-2262. 10.1093/brain/awy147.
17. Robak, L. A., Jansen, I. E., van Rooij, J., Uitterlinden, A. G., Kraaij, R., Jankovic, J., International Parkinson's Disease Genomics, C., Heutink, P., and Shulman, J. M. (2017). Excessive burden of lysosomal storage disorder gene variants in Parkinson's disease. Brain 140, 3191-3203. 10.1093/brain/awx285.
18. Sidransky, E., Nalls, M. A., Aasly, J. O., Aharon-Peretz, J., Annesi, G., Barbosa, E. R., Bar-Shira, A., Berg, D., Bras, J., Brice, A., et al. (2009). Multicenter analysis of glucocerebrosidase mutations in Parkinson's disease. N Engl J Med 361, 1651-1661. 10.1056/NEJMoa0901281.
19. Stojkovska, I., Wani, W. Y., Zunke, F., Belur, N. R., Pavlenko, E. A., Mwenda, N., Sharma, K., Francelle, L., and Mazzulli, J. R. (2022). Rescue of alpha-synuclein aggregation in Parkinson's patient neurons by synergistic enhancement of ER proteostasis and protein trafficking. Neuron 110, 436-451 e411. 10.1016/j.neuron.2021.10.032.
20. Cooper, A. A., Gitler, A. D., Cashikar, A., Haynes, C. M., Hill, K. J., Bhullar, B., Liu, K., Xu, K., Strathearn, K. E., Liu, F., et al. (2006). Alpha-synuclein blocks ER-Golgi traffic and Rab1 rescues neuron loss in Parkinson's models. Science 313, 324-328. 10.1126/science.1129462.
21. Mazzulli, J. R., Xu, Y. H., Sun, Y., Knight, A. L., McLean, P. J., Caldwell, G. A., Sidransky, E., Grabowski, G. A., and Krainc, D. (2011). Gaucher disease glucocerebrosidase and alpha-synuclein form a bidirectional pathogenic loop in synucleinopathies. Cell 146, 37-52. 10.1016/j.cell.2011.06.001.
22. Cuervo, A. M., Stefanis, L., Fredenburg, R., Lansbury, P. T., and Sulzer, D. (2004). Impaired degradation of mutant alpha-synuclein by chaperone-mediated autophagy. Science 305, 1292-1295. 10.1126/science.1101738.
23. Chung, C. Y., Khurana, V., Auluck, P. K., Tardiff, D. F., Mazzulli, J. R., Soldner, F., Baru, V., Lou, Y., Freyzon, Y., Cho, S., et al. (2013). Identification and rescue of alpha-synuclein toxicity in Parkinson patient-derived neurons. Science 342, 983-987. 10.1126/science.1245296.
24. Mazzulli, J. R., Zunke, F., Isacson, O., Studer, L., and Krainc, D. (2016). alpha-synuclein-induced lysosomal dysfunction occurs through disruptions in protein trafficking in human midbrain synucleinopathy models. Proc Natl Acad Sci USA 113, 1931-1936. 10.1073/pnas.1520335113.
25. Chiti, F., Stefani, M., Taddei, N., Ramponi, G., and Dobson, C. M. (2003). Rationalization of the effects of mutations on peptide and protein aggregation rates. Nature 424, 805-808. 10.1038/nature01891.
26. Ciryam, P., Kundra, R., Morimoto, R. I., Dobson, C. M., and Vendruscolo, M. (2015). Supersaturation is a major driving force for protein aggregation in neurodegenerative diseases. Trends Pharmacol Sci 36, 72-77. 10.1016/j.tips.2014.12.004.
27. Fox, A. H., Nakagawa, S., Hirose, T., and Bond, C. S. (2018). Paraspeckles: Where Long Noncoding RNA Meets Phase Separation. Trends Biochem Sci 43, 124-135. 10.1016/j.tibs.2017.12.001.
28. Bond, C. S., and Fox, A. H. (2009). Paraspeckles: nuclear bodies built on long noncoding RNA. J Cell Biol 186, 637-644. 10.1083/jcb.200906113.
29. Zhang, Z., and Carmichael, G. G. (2001). The fate of dsRNA in the nucleus: a p54(nrb)-containing complex mediates the nuclear retention of promiscuously A-to-I edited RNAs. Cell 106, 465-475. 10.1016/s0092-8674(01)00466-4.
30. West, J. A., Mito, M., Kurosaka, S., Takumi, T., Tanegashima, C., Chujo, T., Yanaka, K., Kingston, R. E., Hirose, T., Bond, C., et al. (2016). Structural, super-resolution microscopy analysis of paraspeckle nuclear body organization. J Cell Biol 214, 817-830. 10.1083/jcb.201601071.
31. Souquere, S., Beauclair, G., Harper, F., Fox, A., and Pierron, G. (2010). Highly ordered spatial organization of the structural long noncoding NEAT1 RNAs within paraspeckle nuclear bodies. Mol Biol Cell 21, 4020-4027. 10.1091/mbc.E10-08-0690.
32. Fox, A. H., Lam, Y. W., Leung, A. K., Lyon, C. E., Andersen, J., Mann, M., and Lamond, A. I. (2002). Paraspeckles: a novel nuclear domain. Curr Biol 12, 13-25. 10.1016/s0960-9822(01)00632-7.
33. Knott, G. J., Bond, C. S., and Fox, A. H. (2016). The DBHS proteins SFPQ, NONO and PSPC1: a multipurpose molecular scaffold. Nucleic Acids Res 44, 3989-4004. 10.1093/nar/gkw271.
34. Nishikura, K. (2016). A-to-I editing of coding and non-coding RNAs by ADARs. Nat Rev Mol Cell Biol 17, 83-96. 10.1038/nrm.2015.4.
35. Wagner, R. W., Smith, J. E., Cooperman, B. S., and Nishikura, K. (1989). A double-stranded RNA unwinding activity introduces structural alterations by means of adenosine to inosine conversions in mammalian cells and Xenopus eggs. Proc Natl Acad Sci USA 86, 2647-2651. 10.1073/pnas.86.8.2647.
36. Athanasiadis, A., Rich, A., and Maas, S. (2004). Widespread A-to-I RNA editing of Alu-containing mRNAs in the human transcriptome. PLoS Biol 2, e391. 10.1371/journal.pbio.0020391.
37. Prasanth, K. V., Prasanth, S. G., Xuan, Z., Hearn, S., Freier, S. M., Bennett, C. F., Zhang, M. Q., and Spector, D. L. (2005). Regulating gene expression through RNA nuclear retention. Cell 123, 249-263. 10.1016/j.cell.2005.08.033.
38. Chen, L. L., DeCerbo, J. N., and Carmichael, G. G. (2008). Alu element-mediated gene silencing. EMBO J 27, 1694-1705. 10.1038/emboj.2008.94.
39. Chen, L. L., and Carmichael, G. G. (2009). Altered nuclear retention of mRNAs containing inverted repeats in human embryonic stem cells: functional role of a nuclear noncoding RNA. Mol Cell 35, 467-478. 10.1016/j.molcel.2009.06.027.
40. Elbarbary, R. A., Li, W., Tian, B., and Maquat, L. E. (2013). STAU1 binding 3′ UTR IRAlus complements nuclear retention to protect cells from PKR-mediated translational shutdown. Genes Dev 27, 1495-1510. 10.1101/gad.220962.113.
41. Hu, S. B., Xiang, J. F., Li, X., Xu, Y., Xue, W., Huang, M., Wong, C. C., Sagum, C. A., Bedford, M. T., Yang, L., et al. (2015). Protein arginine methyltransferase CARM1 attenuates the paraspeckle-mediated nuclear retention of mRNAs containing IRAlus. Genes Dev 29, 630-645. 10.1101/gad.257048.114.
42. Hirose, T., Virnicchi, G., Tanigawa, A., Naganuma, T., Li, R., Kimura, H., Yokoi, T., Nakagawa, S., Benard, M., Fox, A. H., and Pierron, G. (2014). NEAT1 long noncoding RNA regulates transcription via protein sequestration within subnuclear bodies. Mol Biol Cell 25, 169-183. 10.1091/mbc.E13-09-0558.
43. Lee, M., Sadowska, A., Bekere, I., Ho, D., Gully, B. S., Lu, Y., Iyer, K. S., Trewhella, J., Fox, A. H., and Bond, C. S. (2015). The structure of human SFPQ reveals a coiled-coil mediated polymer essential for functional aggregation in gene regulation. Nucleic Acids Res 43, 3826-3840. 10.1093/nar/gkv156.
44. Chen, C. X., Cho, D. S., Wang, Q., Lai, F., Carter, K. C., and Nishikura, K. (2000). A third member of the RNA-specific adenosine deaminase gene family, ADAR3, contains both single- and double-stranded RNA binding domains. RNA 6, 755-767. 10.1017/s1355838200000170.
45. Oakes, E., Anderson, A., Cohen-Gadol, A., and Hundley, H. A. (2017). Adenosine Deaminase That Acts on RNA 3 (ADAR3) Binding to Glutamate Receptor Subunit B Pre-mRNA Inhibits RNA Editing in Glioblastoma. J Biol Chem 292, 4326-4335. 10.1074/jbc.M117.779868.
46. Tan, M. H., Li, Q., Shanmugam, R., Piskol, R., Kohler, J., Young, A. N., Liu, K. I., Zhang, R., Ramaswami, G., Ariyoshi, K., et al. (2017). Dynamic landscape and regulation of RNA editing in mammals. Nature 550, 249-254. 10.1038/nature24041.
47. Wu, J., You, Q., Lyu, R., Qian, Y., Tao, H., Zhang, F., Cai, Y., Jiang, N., Zheng, N., Chen, D., and Wu, Z. (2023). Folate metabolism negatively regulates OAS-mediated antiviral innate immunity via ADAR3/endogenous dsRNA pathway. Metabolism 143, 155526. 10.1016/j.metabol.2023.155526.
48. Li, J. B., and Church, G. M. (2013). Deciphering the functions and regulation of brain-enriched A-to-I RNA editing. Nat Neurosci 16, 1518-1522. 10.1038/nn.3539.
49. Hwang, T., Park, C. K., Leung, A. K., Gao, Y., Hyde, T. M., Kleinman, J. E., Rajpurohit, A., Tao, R., Shin, J. H., and Weinberger, D. R. (2016). Dynamic regulation of RNA editing in human brain development and disease. Nat Neurosci 19, 1093-1099. 10.1038/nn.4337.
50. Higuchi, M., Maas, S., Single, F. N., Hartner, J., Rozov, A., Burnashev, N., Feldmeyer, D., Sprengel, R., and Seeburg, P. H. (2000). Point mutation in an AMPA receptor gene rescues lethality in mice deficient in the RNA-editing enzyme ADAR2. Nature 406, 78-81. 10.1038/35017558.
51. Brusa, R., Zimmermann, F., Koh, D. S., Feldmeyer, D., Gass, P., Seeburg, P. H., and Sprengel, R. (1995). Early-onset epilepsy and postnatal lethality associated with an editing-deficient GluR-B allele in mice. Science 270, 1677-1680. 10.1126/science.270.5242.1677.
52. Palladino, M. J., Keegan, L. P., O'Connell, M. A., and Reenan, R. A. (2000). A-to-I pre-mRNA editing in Drosophila is primarily involved in adult nervous system function and integrity. Cell 102, 437-449. 10.1016/s0092-8674(00)00049-0.
53. Li, J. B., Levanon, E. Y., Yoon, J. K., Aach, J., Xie, B., Leproust, E., Zhang, K., Gao, Y., and Church, G. M. (2009). Genome-wide identification of human RNA editing sites by parallel DNA capturing and sequencing. Science 324, 1210-1213. 10.1126/science.1170995.
54. Mladenova, D., Barry, G., Konen, L. M., Pineda, S. S., Guennewig, B., Avesson, L., Zinn, R., Schonrock, N., Bitar, M., Jonkhout, N., et al. (2018). Adar3 Is Involved in Learning and Memory in Mice. Front Neurosci 12, 243. 10.3389/fnins.2018.00243.
55. Li, W., Wu, H., Li, J., Wang, Z., Cai, M., Liu, X., and Liu, G. (2023). Transcriptomic analysis reveals associations of blood-based A-to-I editing with Parkinson's disease. J Neurol. 10.1007/s00415-023-12053-x.
56. Pozdyshev, D. V., Zharikova, A. A., Medvedeva, M. V., and Muronetz, V. I. (2021). Differential Analysis of A-to-I mRNA Edited Sites in Parkinson's Disease. Genes (Basel) 13. 10.3390/genes13010014.
57. Thomas-Jinu, S., Gordon, P. M., Fielding, T., Taylor, R., Smith, B. N., Snowden, V., Blanc, E., Vance, C., Topp, S., Wong, C. H., et al. (2017). Non-nuclear Pool of Splicing Factor SFPQ Regulates Axonal Transcripts Required for Normal Motor Development. Neuron 94, 322-336 e325. 10.1016/j.neuron.2017.03.026.
58. Hogan, A. L., Grima, N., Fifita, J. A., McCann, E. P., Heng, B., Fat, S. C. M., Wu, S., Maharjan, R., Cain, A. K., Henden, L., et al. (2021). Splicing factor proline and glutamine rich intron retention, reduced expression and aggregate formation are pathological features of amyotrophic lateral sclerosis. Neuropathol Appl Neurobiol 47, 990-1003. 10.1111/nan.12749.
59. Miresof, D., Langouet, M., Rio, M., Moutton, S., Siquier-Pernet, K., Bole-Feysot, C., Cagnard, N., Nitschke, P., Gaspar, L., Znidaric, M., et al. (2015). Mutations in NONO lead to syndromic intellectual disability and inhibitory synaptic defects. Nat Neurosci 18, 1731-1736. 10.1038/nn.4169.
60. Ong, S. E., Blagoev, B., Kratchmarova, I., Kristensen, D. B., Steen, H., Pandey, A., and Mann, M. (2002). Stable isotope labeling by amino acids in cell culture, SILAC, as a simple and accurate approach to expression proteomics. Mol Cell Proteomics 1, 376-386. 10.1074/mcp.m200025-mcp200.
61. Soldner, F., Laganiere, J., Cheng, A. W., Hockemeyer, D., Gao, Q., Alagappan, R., Khurana, V., Golbe, L. I., Myers, R. H., Lindquist, S., et al. (2011). Generation of isogenic pluripotent stem cells differing exclusively at two early onset Parkinson point mutations. Cell 146, 318-331. 10.1016/j.cell.2011.06.019.
62. Conway, K. A., Harper, J. D., and Lansbury, P. T. (1998). Accelerated in vitro fibril formation by a mutant alpha-synuclein linked to early-onset Parkinson disease. Nat Med 4, 1318-1320. 10.1038/3311.
63. Cuddy, L. K., Wani, W. Y., Morella, M. L., Pitcairn, C., Tsutsumi, K., Fredriksen, K., Justman, C. J., Grammatopoulos, T. N., Belur, N. R., Zunke, F., et al. (2019). Stress-Induced Cellular Clearance Is Mediated by the SNARE Protein ykt6 and Disrupted by alpha-synuclein. Neuron 104, 869-884 e811. 10.1016/j.neuron.2019.09.001.
64. Cuddy, L. K., Wani, W. Y., Morella, M. L., Pitcairn, C., Tsutsumi, K., Fredriksen, K., Justman, C. J., Grammatopoulos, T. N., Belur, N. R., Zunke, F., et al. (2019). Stress-Induced Cellular Clearance Is Mediated by the SNARE Protein ykt6 and Disrupted by alpha-synuclein. Neuron. 10.1016/j.neuron.2019.09.001.
65. Kim, H. J., Kim, N. C., Wang, Y. D., Scarborough, E. A., Moore, J., Diaz, Z., MacLea, K. S., Freibaum, B., Li, S., Molliex, A., et al. (2013). Mutations in prion-like domains in hnRNPA2B1 and hnRNPA1 cause multisystem proteinopathy and ALS. Nature 495, 467-473. 10.1038/naturel1922.
66. Walsh, I., Martin, A. J., Di Domenico, T., and Tosatto, S. C. (2012). ESpritz: accurate and fast prediction of protein disorder. Bioinformatics 28, 503-509. 10.1093/bioinformatics/btr682.
67. Fiumara, F., Fioriti, L., Kandel, E. R., and Hendrickson, W. A. (2010). Essential role of coiled coils for aggregation and activity of Q/N-rich prions and PolyQ proteins. Cell 143, 1121-1135. 10.1016/j.cell.2010.11.042.
68. Lancaster, A. K., Nutter-Upham, A., Lindquist, S., and King, O. D. (2014). PLAAC: a web and command-line application to identify proteins with prion-like amino acid composition. Bioinformatics 30, 2501-2502. 10.1093/bioinformatics/btu310.
69. Ikai, A. (1980). Thermostability and aliphatic index of globular proteins. J Biochem 88, 1895-1898.
70. Fahn, S., and Cohen, G. (1992). The oxidant stress hypothesis in Parkinson's disease: evidence supporting it. Ann Neurol 32, 804-812. 10.1002/ana.410320616.
71. Sulzer, D. (2007). Multiple hit hypotheses for dopamine neuron loss in Parkinson's disease. Trends Neurosci 30, 244-250. 10.1016/j.tins.2007.03.009.
72. Mazzulli, J. R., Burbulla, L. F., Krainc, D., and Ischiropoulos, H. (2016). Detection of Free and Protein-Bound ortho-Quinones by Near-Infrared Fluorescence. Anal Chem 88, 2399-2405. 10.1021/acs.analchem.5b04420.
73. Sulzer, D., Bogulavsky, J., Larsen, K. E., Behr, G., Karatekin, E., Kleinman, M. H., Turro, N., Krantz, D., Edwards, R. H., Greene, L. A., and Zecca, L. (2000). Neuromelanin biosynthesis is driven by excess cytosolic catecholamines not accumulated by synaptic vesicles. Proc Natl Acad Sci USA 97, 11869-11874. 10.1073/pnas.97.22.11869.
74. Baba, M., Nakajo, S., Tu, P. H., Tomita, T., Nakaya, K., Lee, V. M., Trojanowski, J. Q., and Iwatsubo, T. (1998). Aggregation of alpha-synuclein in Lewy bodies of sporadic Parkinson's disease and dementia with Lewy bodies. Am J Pathol 152, 879-884.
75. Maroteaux, L., Campanelli, J. T., and Scheller, R. H. (1988). Synuclein: a neuron-specific protein localized to the nucleus and presynaptic nerve terminal. J Neurosci 8, 2804-2815. 10.1523/JNEUROSCI.08-08-02804.1988.
76. Koss, D. J., Erskine, D., Porter, A., Palmoski, P., Menon, H., Todd, O. G. J., Leite, M., Attems, J., and Outeiro, T. F. (2022). Nuclear alpha-synuclein is present in the human brain and is modified in dementia with Lewy bodies. Acta Neuropathol Commun 10, 98. 10.1186/s40478-022-01403-x.
77. Schaser, A. J., Osterberg, V. R., Dent, S. E., Stackhouse, T. L., Wakeham, C. M., Boutros, S. W., Weston, L. J., Owen, N., Weissman, T. A., Luna, E., et al. (2019). Alpha-synuclein is a DNA binding protein that modulates DNA repair with implications for Lewy body disorders. Sci Rep 9, 10919. 10.1038/s41598-019-47227-z.
78. Hallacli, E., Kayatekin, C., Nazeen, S., Wang, X. H., Sheinkopf, Z., Sathyakumar, S., Sarkar, S., Jiang, X., Dong, X., Di Maio, R., et al. (2022). The Parkinson's disease protein alpha-synuclein is a modulator of processing bodies and mRNA stability. Cell 185, 2035-2056 e2033. 10.1016/j.cell.2022.05.008.
79. Zunke, F., Moise, A. C., Belur, N. R., Gelyana, E., Stojkovska, I., Dzaferbegovic, H., Toker, N. J., Jeon, S., Fredriksen, K., and Mazzulli, J. R. (2018). Reversible Conformational Conversion of alpha-synuclein into Toxic Assemblies by Glucosylceramide. Neuron 97, 92-107 e110. 10.1016/j.neuron.2017.12.012.
80. Giasson, B. I., Forman, M. S., Higuchi, M., Golbe, L. I., Graves, C. L., Kotzbauer, P. T., Trojanowski, J. Q., and Lee, V. M. (2003). Initiation and synergistic fibrillization of tau and alpha-synuclein. Science 300, 636-640. 10.1126/science.1082324.
81. Chen, S. W., Drakulic, S., Deas, E., Ouberai, M., Aprile, F. A., Arranz, R., Ness, S., Roodveldt, C., Guilliams, T., De-Genst, E. J., et al. (2015). Structural characterization of toxic oligomers that are kinetically trapped during alpha-synuclein fibril formation. Proc Natl Acad Sci USA 112, E1994-2003. 10.1073/pnas.1421204112.
82. Froula, J. M., Castellana-Cruz, M., Anabtawi, N. M., Camino, J. D., Chen, S. W., Thrasher, D. R., Freire, J., Yazdi, A. A., Fleming, S., Dobson, C. M., et al. (2019). Defining alpha-synuclein species responsible for Parkinson's disease phenotypes in mice. J Biol Chem 294, 10392-10406. 10.1074/jbc.RA119.007743.
83. Cremades, N., Cohen, S. I., Deas, E., Abramov, A. Y., Chen, A. Y., Orte, A., Sandal, M., Clarke, R. W., Dunne, P., Aprile, F. A., et al. (2012). Direct observation of the interconversion of normal and toxic forms of alpha-synuclein. Cell 149, 1048-1059. 10.1016/j.cell.2012.03.037.
84. Fusco, G., Chen, S. W., Williamson, P. T. F., Cascella, R., Perni, M., Jarvis, J. A., Cecchi, C., Vendruscolo, M., Chiti, F., Cremades, N., et al. (2017). Structural basis of membrane disruption and cellular toxicity by alpha-synuclein oligomers. Science 358, 1440-1443. 10.1126/science.aan6160.
85. Melcher, T., Maas, S., Herb, A., Sprengel, R., Higuchi, M., and Seeburg, P. H. (1996). RED2, a brain-specific member of the RNA-specific adenosine deaminase family. J Biol Chem 271, 31795-31798. 10.1074/jbc.271.50.31795.
86. Kumar, M., and Carmichael, G. G. (1997). Nuclear antisense RNA induces extensive adenosine modifications and nuclear retention of target transcripts. Proc Natl Acad Sci USA 94, 3542-3547. 10.1073/pnas.94.8.3542.
87. Schenck, A., Bardoni, B., Langmann, C., Harden, N., Mandel, J. L., and Giangrande, A. (2003). CYFIP/Sra-1 controls neuronal connectivity in Drosophila and links the Rac1 GTPase pathway to the fragile X protein. Neuron 38, 887-898. 10.1016/s0896-6273(03)00354-4.
88. Abekhoukh, S., and Bardoni, B. (2014). CYFIP family proteins between autism and intellectual disability: links with Fragile X syndrome. Front Cell Neurosci 8, 81. 10.3389/fncel.2014.00081.
89. Jockusch, W. J., Speidel, D., Sigler, A., Sorensen, J. B., Varoqueaux, F., Rhee, J. S., and Brose, N. (2007). CAPS-1 and CAPS-2 are essential synaptic vesicle priming proteins. Cell 131, 796-808. 10.1016/j.cell.2007.11.002.
90. Nicholas, A., de Magalhaes, J. P., Kraytsberg, Y., Richfield, E. K., Levanon, E. Y., and Khrapko, K. (2010). Age-related gene-specific changes of A-to-I mRNA editing in the human brain. Mech Ageing Dev 131, 445-447. 10.1016/j.mad.2010.06.001.
91. Burke, R. E., and O'Malley, K. (2013). Axon degeneration in Parkinson's disease. Exp Neurol 246, 72-83. 10.1016/j.expneurol.2012.01.011.
92. Paz-Yaacov, N., Levanon, E. Y., Nevo, E., Kinar, Y., Harmelin, A., Jacob-Hirsch, J., Amariglio, N., Eisenberg, E., and Rechavi, G. (2010). Adenosine-to-inosine RNA editing shapes transcriptome diversity in primates. Proc Natl Acad Sci USA 107, 12174-12179. 10.1073/pnas.1006183107.
93. Veliz, E. A., Easterwood, L. M., and Beal, P. A. (2003). Substrate analogues for an RNA-editing adenosine deaminase: mechanistic investigation and inhibitor design. J Am Chem Soc 125, 10867-10876. 10.1021/ja029742d.
94. Spremulli, E. N., Crabtree, G. W., Dexter, D. L., Chu, S. H., Farineau, D. M., Ghoda, L. Y., McGowan, D. L., Diamond, I., Parks, R. E., Jr., and Calabresi, P. (1982). Biochemical pharmacology and toxicology of 8-azaadenosine alone and in combination with 2′-deoxycoformycin (pentostatin). Biochem Pharmacol 31, 2415-2421. 10.1016/0006-2952(82)90538-x.
95. Montgomery, J. A., Elliott, R. D., and Thomas, H. J. (1975). The synthesis and evaluation of azapurine nucleosides as cytotoxic agents. Ann N Y Acad Sci 255, 292-305. 10.1111/j.1749-6632.1975.tb29237.x.
96. Bennett, L. L., Jr., and Allan, P. W. (1976). Metabolism and metabolic effects of 8-azainosine and 8-azaadenosine. Cancer Res 36, 3917-3923.
97. Zipeto, M. A., Court, A. C., Sadarangani, A., Delos Santos, N. P., Balaian, L., Chun, H. J., Pineda, G., Morris, S. R., Mason, C. N., Geron, I., et al. (2016). ADAR1 Activation Drives Leukemia Stem Cell Self-Renewal by Impairing Let-7 Biogenesis. Cell Stem Cell 19, 177-191. 10.1016/j.stem.2016.05.004.
98. Ramirez-Moya, J., Baker, A. R., Slack, F. J., and Santisteban, P. (2020). ADAR1-mediated RNA editing is a novel oncogenic process in thyroid cancer and regulates miR-200 activity. Oncogene 39, 3738-3753. 10.1038/s41388-020-1248-x.
99. Cottrell, K. A., Torres, L. S., Dizon, M. G., and Weber, J. D. (2021). 8-azaadenosine and 8-chloroadenosine are not selective inhibitors of ADAR. Cancer Res Commun 1, 56-64. 10.1158/2767-9764.crc-21-0027.
100. Garncarz, W., Tariq, A., Handl, C., Pusch, O., and Jantsch, M. F. (2013). A high-throughput screen to identify enhancers of ADAR-mediated RNA-editing. RNA Biol 10, 192-204. 10.4161/rna.23208.
101. Wang, Y., Havel, J., and Beal, P. A. (2015). A Phenotypic Screen for Functional Mutants of Human Adenosine Deaminase Acting on RNA 1. ACS Chem Biol 10, 2512-2519. 10.1021/acschembio.5b00711.
102. Ray, D., Kazan, H., Cook, K. B., Weirauch, M. T., Najafabadi, H. S., Li, X., Gueroussov, S., Albu, M., Zheng, H., Yang, A., et al. (2013). A compendium of RNA-binding motifs for decoding gene regulation. Nature 499, 172-177. 10.1038/naturel2311.
103. Copley, K. E., and Shorter, J. (2022). Flying under the radar: TMEM106B(120-254) fibrils break out in diverse neurodegenerative disorders. Cell 185, 1290-1292. 10.1016/j.cell.2022.03.032.
104. Schweighauser, M., Arseni, D., Bacioglu, M., Huang, M., Lovestam, S., Shi, Y., Yang, Y., Zhang, W., Kotecha, A., Garringer, H. J., et al. (2022). Age-dependent formation of TMEM106B amyloid filaments in human brains. Nature 605, 310-314. 10.1038/s41586-022-04650-z.
105. Jiang, Y. X., Cao, Q., Sawaya, M. R., Abskharon, R., Ge, P., DeTure, M., Dickson, D. W., Fu, J. Y., Ogorzalek Loo, R. R., Loo, J. A., and Eisenberg, D. S. (2022). Amyloid fibrils in FTLD-TDP are composed of TMEM106B and not TDP-43. Nature 605, 304-309. 10.1038/s41586-022-04670-9.
106. Chang, A., Xiang, X., Wang, J., Lee, C., Arakhamia, T., Simjanoska, M., Wang, C., Carlomagno, Y., Zhang, G., Dhingra, S., et al. (2022). Homotypic fibrillization of TMEM106B across diverse neurodegenerative diseases. Cell 185, 1346-1355 e1315. 10.1016/j.cell.2022.02.026.
107. Rebagliati, M. R., and Melton, D. A. (1987). Antisense RNA injections in fertilized frog eggs reveal an RNA duplex unwinding activity. Cell 48, 599-605. 10.1016/0092-8674(87)90238-8.
108. Stellos, K., Gatsiou, A., Stamatelopoulos, K., Perisic Matic, L., John, D., Lunella, F. F., Jae, N., Rossbach, O., Amrhein, C., Sigala, F., et al. (2016). Adenosine-to-inosine RNA editing controls cathepsin S expression in atherosclerosis by enabling HuR-mediated post-transcriptional regulation. Nat Med 22, 1140-1150. 10.1038/nm.4172.
109. Nishikura, K. (2010). Functions and regulation of RNA editing by ADAR deaminases. Annu Rev Biochem 79, 321-349. 10.1146/annurev-biochem-060208-105251.
110. Dawson, T. M., Ko, H. S., and Dawson, V. L. (2010). Genetic animal models of Parkinson's disease. Neuron 66, 646-661. 10.1016/j.neuron.2010.04.034.
111. Nakagawa, S., Naganuma, T., Shioi, G., and Hirose, T. (2011). Paraspeckles are subpopulation-specific nuclear bodies that are not essential in mice. J Cell Biol 193, 31-39. 10.1083/jcb.201011110.
112. Harrison, A. F., and Shorter, J. (2017). RNA-binding proteins with prion-like domains in health and disease. Biochem J 474, 1417-1438. 10.1042/BCJ20160499.
113. Sakurai, M., Shiromoto, Y., Ota, H., Song, C., Kossenkov, A. V., Wickramasinghe, J., Showe, L. C., Skordalakes, E., Tang, H. Y., Speicher, D. W., and Nishikura, K. (2017). ADAR1 controls apoptosis of stressed cells by inhibiting Staufen1-mediated mRNA decay. Nat Struct Mol Biol 24, 534-543. 10.1038/nsmb.3403.
114. Stojkovska, I., and Mazzulli, J. R. (2021). Detection of pathological alpha-synuclein aggregates in human iPSC-derived neurons and tissue. STAR Protoc 2, 100372. 10.1016/j.xpro.2021.100372.
115. Lionnet, T., Czaplinski, K., Darzacq, X., Shav-Tal, Y., Wells, A. L., Chao, J. A., Park, H. Y., de Turris, V., Lopez-Jones, M., and Singer, R. H. (2011). A transgenic mouse for in vivo detection of endogenous labeled mRNA. Nat Methods 8, 165-170. 10.1038/nmeth.1551.
116. Polinski, N. K., Volpicelli-Daley, L. A., Sortwell, C. E., Luk, K. C., Cremades, N., Gottler, L. M., Froula, J., Duffy, M. F., Lee, V. M. Y., Martinez, T. N., and Dave, K. D. (2018). Best Practices for Generating and Using Alpha-synuclein Pre-Formed Fibrils to Model Parkinson's Disease in Rodents. J Parkinsons Dis 8, 303-322. 10.3233/JPD-171248.
117. Gruber, A. R., Lorenz, R., Bernhart, S. H., Neubock, R., and Hofacker, I. L. (2008). The Vienna RNA websuite. Nucleic Acids Res 36, W70-74. 10.1093/nar/gkn188.
118. Dzyubenko, E., Rozenberg, A., Hermann, D. M., and Faissner, A. (2016). Colocalization of synapse marker proteins evaluated by STED-microscopy reveals patterns of neuronal synapse distribution in vitro. J Neurosci Methods 273, 149-159. 10.1016/j.jneumeth.2016.09.001.
119. Ciryam, P., Tartaglia, G. G., Morimoto, R. I., Dobson, C. M., and Vendruscolo, M. (2013). Widespread aggregation and neurodegenerative diseases are associated with supersaturated proteins. Cell Rep 5, 781-790. 10.1016/j.celrep.2013.09.043.
120. Walsh, I., Seno, F., Tosatto, S. C., and Trovato, A. (2014). PASTA 2.0: an improved server for protein aggregation prediction. Nucleic Acids Res 42, W301-307. 10.1093/nar/gku399.
121. Li, H., and Durbin, R. (2009). Fast and accurate short read alignment with Burrows-Wheeler transform. Bioinformatics 25, 1754-1760. 10.1093/bioinformatics/btp324.
122. McKenna, A., Hanna, M., Banks, E., Sivachenko, A., Cibulskis, K., Kernytsky, A., Garimella, K., Altshuler, D., Gabriel, S., Daly, M., and DePristo, M. A. (2010). The Genome Analysis Toolkit: a MapReduce framework for analyzing next-generation DNA sequencing data. Genome Res 20, 1297-1303. 10.1101/gr.107524.110.
123. Danecek, P., Bonfield, J. K., Liddle, J., Marshall, J., Ohan, V., Pollard, M. O., Whitwham, A., Keane, T., McCarthy, S. A., Davies, R. M., and Li, H. (2021). Twelve years of SAMtools and BCFtools. Gigascience 10. 10.1093/gigascience/giab008.
124. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
125. Wang, K., Li, M., and Hakonarson, H. (2010). ANNOVAR: functional annotation of genetic variants from high-throughput sequencing data. Nucleic Acids Res 38, e164. 10.1093/nar/gkg603.
126. Lo Giudice, C., Tangaro, M. A., Pesole, G., and Picardi, E. (2020). Investigating RNA editing in deep transcriptome datasets with REDItools and REDIportal. Nat Protoc 15, 1098-1131. 10.1038/s41596-019-0279-7.
127. Giasson, B. I., Duda, J. E., Quinn, S. M., Zhang, B., Trojanowski, J. Q., and Lee, V. M. (2002). Neuronal alphα-synucleinopathy with severe movement disorder in mice expressing A53T human alphα-synuclein. Neuron 34, 521-533. 10.1016/s0896-6273(02)00682-7.

Sequence

| SEQ ID | |
|---|---|
| NO: | Sequence |
| 1 | uuaaagaaaauucucaggccaggcucaauggcucaugccuauaauuguagugcuuugggaggcugag |
| | cugggaggaucgcuuaagaccagccuaggcaacagagugagauccugucucuacaaaaacaaaaauuu |
| | ucuuggcucaccgcaaccuccgccucccagauuaaagugauucuccugccucaaccuccugaguagcu |
| | gggauuacaggcaugcgccaccaugccgggcgaauuuuguauuuuuaauagagacaggguuucucug |
| | uguuggucaggcuggucucaaacucccaaccucaggugauccgcccaccucagccucucagagugcug |
| | ggauuacagaugugagccacugcaccuggcaaaaaaaaaauuuuuuuuuaauuugucagguguggug |
| | augcaugcauguagucccaaauacuuaggaggcugagguggaaggaucacuugagcccaggagguca |
| | aggcaacagugagcugugaucaugccacugcacuccagucugggugacacagugagacccugucucu |
| | uaaaaaaaaaaaaagaaagaaaaaaaauuauucuuuagguaauuucacuuggacauauucauguauau |
| | ggaguuugucagcugacucaggcauuuacccuaaagaucaaaucuguuuucaaaaauuggcauuggc |
| | caggcacggugacccacaccuguaaucccagcacuuugggaggccgagggggcggaucacgagguca |
| | gggguucaagaccagccuggccaacauagugaaaccccgucucuacuaaaaauacaaaaaauuagcug |
| | ggcaugguggugggcgccuguaaucccagcuacuugagaggcucaggcaggagaaucgcuugaaccu |
| | gggaguggagguugcagugagccgagauugcaccauuguauuccaccccgggugacagugcaagacu |
| | ccaucucaaaaaaaaaaaaaaaauuuggcaucauu |
| 2 | uuaaagaaaauucucaggccaggcucaauggcucaugccuauaauuguagugcuuugggaggcugag |
| | cugggaggaucgcuuaagaccagccuIggcaacagagugagauccugucucuacaaaaacaaaaauuu |
| | ucuuggcucaccgcaaccuccgccucccagauuaaagugauucuccugccucaaccuccugaguagcu |
| | gggauuacaggcaugcgccaccaugccgggcgaauuuuguauuuuuaauagagacaggguuucucug |
| | uguuggucaggcuggucucaaacucccaaccucaggugauccgcccaccucagccucucagagugcug |
| | ggauuacagaugugagccacugcaccuggcaaaaaaaaaauuuuuuuuuaauuugucagguguggug |
| | augcaugcauguagucccaaauacuuaggaggcugagguggaaggaucacuugagcccaggagguca |
| | aggcaacagugagcugugaucaugccacugcacuccagucugggugacacagugagacccugucucuu |
| | aaaaaaaaaaaaagaaagaaaaaaaauuauucuuuagguaauuucacuuggacauauucauguauaug |
| | gaguuugucagcugacucaggcauuuacccuaaagaucaaaucuguuuucaaaaauuggcauuggcca |
| | ggcacggugacccacaccuguaaucccagcacuuugggaggccgagggggcggaucacgaggucagg |
| | gguucaagaccagccuggccaacauagugaaaccccgucucuacuaaaaauacaaaaaauuagcugggc |
| | augguggugggcgccuguaaucccagcuacuugagaggcucaggcaggagaaucgcuugaaccuggg |
| | aguggagguugcagugagccgagauugcaccauuguauuccaccccgggugacagugcaagacucca |
| | ucucaaaaaaaaaaaaaaaauuuggcaucauu |
| 3 | agccuaggcaacagagugagauccugucuc |
| 4 | agccu [symbol] ggc [symbol] c [symbol] g [symbol] gug [symbol] gauccugucuc |
| 5 | 5'-2MOErA/i2MOErG/i2MOErA/i2MOErA/i2MOErA/ |
| | CACCTGCGGC/i2MOErG/i2MOErG/i2MOErC/i2MOErT/i2MOErC- |
| | 3' |

What is claimed:

1. A method of treating a neurodegenerative disease in a subject in need thereof, the method comprising administering an inhibitor of Adenosine (A) to Inosine (I) RNA editing to the subject.

2. The method of claim 1, wherein the inhibitor comprises at least one of: an ADAR1 inhibitor; an ADAR2 inhibitor; and an ADAR3 activator.

3. The method of claim 2, wherein the ADAR1 inhibitor inhibits at least one of ADAR1 expression and activity.

4. The method of claim 3, wherein the ADAR1 inhibitor inhibits ADAR1 activity.

5. The method of claim 3, wherein the ADAR1 inhibitor is a small-molecule drug.

6. The method of claim 5, wherein the ADAR1 inhibitor comprises 8-aza-adenosine (8-aza).

7. The method of claim 6, wherein the 8-aza is administered at a sub-toxic dose.

8. The method of claim 7, wherein the 8-aza is administered at less than about 1 μM.

9. The method of claim 7, wherein the 8-aza is administered at between about 0.05 μM and about 1 μM.

10. The method of claim 9, wherein the 8-aza is administered at about 200 nM.

11. The method of claim 1, wherein the neurodegenerative disease is selected from the group consisting of Parkinson's disease, Alzheimer's disease, dementia with Lewy bodies and multiple system atrophy.

12. A method of preventing or dissolving pathological aggregates comprising at least one of NonPOU Domain-Containing Octamer-Binding Protein (NONO) and Splicing Factor, Proline- and Glutamine-Rich (SFPQ) in a cell, the method comprising contacting the cell with an inhibitor of adenosine (A) to inosine (I) RNA editing.

13. The method of claim 12, wherein the inhibitor comprises at least one of: an ADAR1 inhibitor; an ADAR2 inhibitor; and an ADAR3 activator.

14. The method of claim 13, wherein the ADAR1 inhibitor inhibits at least one of ADAR1 expression and activity.

15. The method of claim 14, wherein the ADAR1 inhibitor comprises 8-aza-adenosine (8-aza).

16. The method of claim 15, wherein the cell is contacted with less than about 1 μM 8-aza.

17. The method of claim 16, wherein the cell is contacted with about 200 nM 8-aza.

18. The method of claim 12 wherein the cell is an induced pluripotent stem neuronal (iPSn) cell derived from a subject having a neurodegenerative disease.

19. A method of treating a neurodegenerative disease or a neurodevelopmental disorder in a subject in need thereof, the method comprising administering an inhibitor of NEAT1_2 to the subject.

20. The method of claim 19 wherein the NEAT1_2 inhibitor is 8-aza or an antisense oligonucleotide.
